SEQUENCE LISTING (7 sequences)

SEQ ID NO: 1 - DNA, 18 bp, synthetic construct
  source 1..18: mol_type = other DNA
  modified_base 1: mod_base = OTHER; note = 2'-O-methyl adenine
  modified_base 2: mod_base = OTHER; note = 2'-4' locked nucleic acid
  modified_base 3: mod_base = OTHER; note = phosphorothioate linkage 2'-4' locked nucleic acid
  modified_base 4: mod_base = OTHER; note = phosphorothioate linkage 2'-4' locked nucleic acid
  modified_base 5: mod_base = OTHER; note = phosphorothioate linkage 2'-4' locked nucleic acid
  modified_base 6: mod_base = OTHER; note = phosphorothioate linkage 2'-4' locked nucleic acid
  modified_base 7..8: mod_base = OTHER; note = phosphorothioate linkage
  modified_base 9: mod_base = OTHER; note = phosphorothioate linkage 5-hydroxy Cytidine base
  modified_base 10: mod_base = OTHER; note = phosphorothioate linkage
  modified_base 11..12: mod_base = OTHER; note = phosphorothioate linkage 5-methyl Cytidine base
  modified_base 13: mod_base = OTHER; note = phosphorothioate linkage
  modified_base 14: mod_base = OTHER; note = phosphorothioate linkage 5-methyl Cytidine base
  modified_base 15: mod_base = OTHER; note = phosphorothioate linkage 2'-4' locked nucleic acid
  modified_base 16: mod_base = OTHER; note = phosphorothioate linkage 2'-4' locked nucleic acid
  modified_base 17: mod_base = OTHER; note = phosphorothioate linkage 2'-4' locked nucleic acid
  modified_base 18: mod_base = OTHER; note = phosphorothioate linkage 5-methyl Cytidine base
  modified_base 18: mod_base = OTHER; note = cyclopropyl 2'-4' locked nucleic acid
agataaaacgccgcagac

SEQ ID NO: 2 - RNA, 19 nt, synthetic construct
  source 1..19: mol_type = other RNA
  modified_base 1: mod_base = cm
  modified_base 2: mod_base = OTHER; note = phosphorothioate linkage, 2'-O-methyl cytosine
  modified_base 3: mod_base = OTHER; note = phosphorothioate linkage, 2'-O-methyl guanine
  modified_base 4: mod_base = um
  modified_base 5: mod_base = OTHER; note = 2'-fluoro guanine
  modified_base 6: mod_base = um
  modified_base 7: mod_base = OTHER; note = 2'-fluoro guanine
  modified_base 8: mod_base = OTHER; note = 2'-fluoro cytosine
  modified_base 9: mod_base = OTHER; note = 2'-fluoro adenine
  modified_base 10: mod_base = cm
  modified_base 11: mod_base = um
  modified_base 12: mod_base = um
  modified_base 13: mod_base = cm
  modified_base 14: mod_base = gm
  modified_base 15: mod_base = cm
  modified_base 16: mod_base = um
  modified_base 17: mod_base = um
  modified_base 18: mod_base = cm
  modified_base 19: mod_base = OTHER; note = 2'-O-methyl adenine
ccgtgtgcacttcgcttca

SEQ ID NO: 3 - RNA, 21 nt, synthetic construct
  source 1..21: mol_type = other RNA
  modified_base 1: mod_base = um
  modified_base 2: mod_base = OTHER; note = phosphorothioate linkage, 2'-fluoro guanine
  modified_base 3: mod_base = OTHER; note = phosphorothioate linkage, 2'-O-methyl adenine
  modified_base 4: mod_base = OTHER; note = 2'-O-methyl adenine
  modified_base 5: mod_base = OTHER; note = 2'-fluoro guanine
  modified_base 6: mod_base = cm
  modified_base 7: mod_base = gm
  modified_base 8: mod_base = OTHER; note = 2'-fluoro adenine
  modified_base 9: mod_base = OTHER; note = 2'-O-methyl adenine
  modified_base 10: mod_base = gm
  modified_base 11: mod_base = um
  modified_base 12: mod_base = gm
  modified_base 13: mod_base = cm
  modified_base 14: mod_base = OTHER; note = 2'-fluoro adenine
  modified_base 15: mod_base = cm
  modified_base 16: mod_base = OTHER; note = 2'-O-methyl adenine
  modified_base 17: mod_base = OTHER; note = 2'-fluoro cytosine
  modified_base 18: mod_base = gm
  modified_base 19: mod_base = gm
  modified_base 20: mod_base = OTHER; note = phosphorothioate linkage, 2'-O-methyl uracil
  modified_base 21: mod_base = OTHER; note = phosphorothioate linkage, 2'-O-methyl cytosine
tgaagcgaagtgcacacggtc

SEQ ID NO: 4 - RNA, 19 nt, synthetic construct
  source 1..19: mol_type = other RNA
  modified_base 1: mod_base = gm
  modified_base 2: mod_base = OTHER; note = phosphorothioate linkage 2'-O-methyl uracil
  modified_base 3: mod_base = OTHER; note = phosphorothioate linkage, 2'-O-methyl guanine
  modified_base 4: mod_base = gm
  modified_base 5: mod_base = OTHER; note = 2'-fluoro uracil
  modified_base 6: mod_base = gm
  modified_base 7: mod_base = OTHER; note = 2'-fluoro guanine
  modified_base 8: mod_base = OTHER; note = 2'-fluoro adenine
  modified_base 9: mod_base = OTHER; note = 2'-fluoro cytosine
  modified_base 10: mod_base = um
  modified_base 11: mod_base = um
  modified_base 12: mod_base = cm
  modified_base 13: mod_base = um
  modified_base 14: mod_base = cm
  modified_base 15: mod_base = um
  modified_base 16: mod_base = cm
  modified_base 17..18: mod_base = OTHER; note = 2'-O-methyl adenine
  modified_base 19: mod_base = um
gtggtggacttctctcaat

SEQ ID NO: 5 - RNA, 21 nt, synthetic construct
  source 1..21: mol_type = other RNA
  modified_base 1: mod_base = OTHER; note = 2'-O-methyl adenine
  modified_base 2: mod_base = OTHER; note = phosphorothioate linkage, 2'-fluoro uracil
  modified_base 3: mod_base = OTHER; note = phosphorothioate linkage, 2'-O-methyl uracil
  modified_base 4: mod_base = gm
  modified_base 5: mod_base = OTHER; note = 2'-O-methyl adenine
  modified_base 6: mod_base = OTHER; note = 2'-fluoro guanine
  modified_base 7: mod_base = OTHER; note = 2'-O-methyl adenine
  modified_base 8: mod_base = gm
  modified_base 9..10: mod_base = OTHER; note = 2'-O-methyl adeninie
  modified_base 11: mod_base = gm
  modified_base 12: mod_base = um
  modified_base 13: mod_base = cm
  modified_base 14: mod_base = OTHER; note = 2'-fluoro cytosine
  modified_base 15: mod_base = OTHER; note = 2'-O-methyl adenine
  modified_base 16: mod_base = OTHER; note = 2'-fluoro cytosine
  modified_base 17: mod_base = cm
  modified_base 18: mod_base = OTHER; note = 2'-O-methyl adenine
  modified_base 19: mod_base = cm
  modified_base 20..21: mod_base = OTHER; note = phosphorothioate linkage 2'-O-methyl adenine
attgagagaagtccaccacaa

SEQ ID NO: 6 - RNA, 19 nt, synthetic construct
  source 1..19: mol_type = other RNA
  modified_base 1: mod_base = gm
  modified_base 2: mod_base = OTHER; note = phosphorothioate linkage 2'-O-methyl uracil
  modified_base 3: mod_base = OTHER; note = phosphorothioate linkage 2'-O-methyl guanine
  modified_base 4: mod_base = gm
  modified_base 5: mod_base = OTHER; note = 2'-fluoro guanine
  modified_base 6: mod_base = gm
  modified_base 7: mod_base = OTHER; note = 2'-fluoro guanine
  modified_base 8: mod_base = OTHER; note = 2'-fluoro adenine
  modified_base 9: mod_base = OTHER; note = 2'-fluoro cytosine
  modified_base 10..11: mod_base = um
  modified_base 12: mod_base = cm
  modified_base 13: mod_base = um
  modified_base 14: mod_base = cm
  modified_base 15: mod_base = um
  modified_base 16: mod_base = cm
  modified_base 17..18: mod_base = OTHER; note = 2'-O-methyl adenine
  modified_base 19: mod_base = um
gtggtggacttctctcaat

SEQ ID NO: 7 - RNA, 21 nt, synthetic construct
  source 1..21: mol_type = other RNA
  modified_base 1: mod_base = OTHER; note = 2'-O-methyl adenine
  modified_base 2: mod_base = OTHER; note = phosphorothioate linkage 2'-fluoro uracil
  modified_base 3: mod_base = OTHER; note = phosphorothioate 2'-O-methyl uracil
  modified_base 4: mod_base = gm
  modified_base 5: mod_base = OTHER; note = 2'-O-methyl adenine
  modified_base 6: mod_base = OTHER; note = 2'-fluoro guanine
  modified_base 7: mod_base = OTHER; note = 2'-O-methyl adenine
  modified_base 8: mod_base = gm
  modified_base 9..10: mod_base = OTHER; note = 2'-O-methyl adenine
  modified_base 11: mod_base = gm
  modified_base 12: mod_base = um
  modified_base 13: mod_base = cm
  modified_base 14: mod_base = OTHER; note = 2'-fluoro cytosine
  modified_base 15: mod_base = OTHER; note = 2'-O-methyl adenine
  modified_base 16: mod_base = OTHER; note = 2'-fluoro cytosine
  modified_base 17: mod_base = cm
  modified_base 18: mod_base = OTHER; note = 2'-O-methyl adenine
  modified_base 19: mod_base = cm
  modified_base 20: mod_base = OTHER; note = phosphorothioate linkage 2'-O-methyl guanine
  modified_base 21: mod_base = OTHER; note = phosphorothioate linkage, 2'-O-methyl adenine
attgagagaagtccaccacga

(19) United States
(12) Patent Application Publication
(10) Pub. No.: US 2026/0226470 A1
(43) Pub. Date: Aug. 6, 2026

(54) HEPATITIS B COMBINATION THERAPIES

(71) Applicant: Aligos Therapeutics, Inc., South San Francisco, CA (US)

(72) Inventors: Yannick Debing, Bilzen (BE); Sandrine Vendeville, Brussels (BE); Megan Elizabeth Fitzgerald, San Francisco, CA (US); Hannah Vanrusselt, Aarschot (BE); Julian Alexander Symons, San Carlos, CA (US)

(21) Appl. No.: 19/150,867

(22) PCT Filed: Jan. 24, 2024
(86) PCT No.: PCT/US2024/012770

§ 371 Date: Jul. 24, 2025

Related U.S. Application Data
(60) Provisional application No. 63/481,763, filed on Jan. 26, 2023.

(51) Int. Cl. C12N 15/113; A61K 31/4025; A61K 31/422; A61K 31/4439; A61K 31/445; A61K 31/472; A61K 31/4985; A61K 31/506; A61K 31/519; A61K 31/5377; A61K 31/554; A61K 31/675; A61K 31/712; A61K 31/7125; A61K 31/713; A61K 45/06; A61P 31/20

(52) U.S. Cl. CPC C12N 15/1133; A61K 31/4025; A61K 31/422; A61K 31/4439; A61K 31/445; A61K 31/472; A61K 31/4985; A61K 31/506; A61K 31/519; A61K 31/5377; A61K 31/554; A61K 31/675; A61K 31/712; A61K 31/7125; A61K 31/713; A61K 45/06; A61P 31/20; C12N 2310/11; C12N 2310/14; C12N 2310/315; C12N 2310/321; C12N 2310/322

(57) ABSTRACT

Provided herein are methods of treating a hepatitis B viral and/or a hepatitis D viral infection in a subject in need thereof. In some embodiments, the methods include administering an effective amount of a first agent selected from the group consisting of (i) an HBV surface antigen (HBsAg) reducing agent, or a pharmaceutically acceptable salt thereof, or and (ii) a capsid assembly modulator (CAM), or a pharmaceutically acceptable salt thereof, to the subject followed by administering an effective amount of a second agent selected from the group consisting of (i) a CAM, or a pharmaceutically acceptable salt thereof, and (ii) an HBV surface antigen (HBsAg) reducing agent, or a pharmaceutically acceptable salt thereof, to the subject, wherein when the first agent is an HBsAg reducing agent or a pharmaceutically acceptable salt thereof, the second agent is a CAM or a pharmaceutically acceptable salt thereof; and wherein when the first agent is a CAM or a pharmaceutically acceptable salt thereof, the second agent is an HBsAg reducing agent or a pharmaceutically acceptable salt thereof; and wherein the initial administration of the second agent is after a delay period following the initial administration of the first agent.

### INCORPORATION BY REFERENCE TO ANY PRIORITY APPLICATIONS

Any and all applications for which a foreign or domestic priority claim is identified, for example, in the Application Data Sheet or Request as filed with the present application, are hereby incorporated by reference under 37 CFR 1.57, and Rules 4.18 and 20.6, including U.S. Provisional Application No. 63/481,763, filed Jan. 26, 2023. REFERENCE TO SEQUENCE LISTING

The present application is being filed along with a sequence listing in electronic format. The sequence listing is provided as a file entitled “ALIG087WO3 SEQ List.xml”, created Jan. 22, 2024, which is 71 KB in size. The information in the electronic format of the sequence listing is incorporated herein by reference in its entirety.

### BACKGROUND

The hepatitis B virus (HBV) is a DNA virus and a member of the Hepadnaviridae family. HBV infects more than 300 million worldwide, and is a causative agent of liver cancer and liver disease such as chronic hepatitis, cirrhosis, and hepatocellular carcinoma. Although there are approved drugs for treating HBV, by either boosting the immune system or slowing down the replication of the virus, they only rarely result in functional cure of chronic hepatitis B patients.

The hepatitis D virus (HDV) is a DNA virus, also in the Hepadnaviridae family of viruses. HDV can propagate only in the presence of HBV. The routes of transmission of HDV are similar to those for HBV. Transmission of HDV can occur either via simultaneous infection with HBV (coinfection) or in addition to chronic hepatitis B or hepatitis B carrier state (superinfection). Both superinfection and coinfection with HDV results in more severe complications compared to infection with HBV alone. These complications include a greater likelihood of experiencing liver failure in acute infections and a rapid progression to liver cirrhosis, with an increased risk of developing liver cancer in chronic infections. In combination with hepatitis B, hepatitis D has the highest fatality rate of all the hepatitis infections, at 20%. There is currently no cure or vaccine for hepatitis D.

### SUMMARY

The present invention provides compounds, methods and compositions for preventing, treating and/or curing hepatitis B virus (HBV) and/or hepatitis D virus (HDV) infection in a host, or reducing the activity of HBV and/or HDV in the host. Provided herein are methods of treating hepatitis B viral and/or hepatitis D viral infection in a subject in need thereof. In some embodiments, the method includes administering an effective amount of a first agent selected from the group consisting of (i) an HBV surface antigen (HBsAg) reducing agent, or a pharmaceutically acceptable salt thereof, and (ii) a capsid assembly modulator (CAM), or a pharmaceutically acceptable salt thereof, to the subject, followed by administering an effective amount of a second agent selected from the group consisting of (i) a CAM, or a pharmaceutically acceptable salt thereof, and (ii) an HBsAg reducing agent, or a pharmaceutically acceptable salt thereof, to the subject, wherein when the first agent is an HBsAg reducing agent or a pharmaceutically acceptable salt thereof, the second agent is a CAM or a pharmaceutically acceptable salt thereof, and wherein when the first agent is a CAM or a pharmaceutically acceptable salt thereof, the second agent is an HBsAg reducing agent or a pharmaceutically acceptable salt thereof, and wherein initial administration of the second agent is after a delay period following initial administration of the first agent.

In some embodiments, the method is a method of treating an HBV infection. In some embodiments, the method is a method of treating an HDV infection.

In some embodiments, the first agent is an HBsAg reducing agent or a pharmaceutically acceptable salt thereof; and the second agent is a CAM or a pharmaceutically acceptable salt thereof. In some embodiments, the first agent is a CAM or a pharmaceutically acceptable salt thereof; and the second agent is an HBsAg reducing agent or a pharmaceutically acceptable salt thereof.

In some embodiments, the CAM is a Class A CAM (CAM-A). In some embodiments, the CAM is a Class E CAM (CAM-E). In some embodiments, the HBsAg reducing agent is a small interfering RNA (siRNA). In some embodiments, the HBsAg reducing agent is an antisense oligonucleotide (ASO).

In some embodiments, the CAM is a fused pyrazole compound, a fused pyrimidone compound, or a pyrrole compound. In some embodiments, the CAM is selected from the group consisting of: N-methyl-4-[(11R)-12-[4-bromo-3-(trifluoromethyl)benzoyl]-5-(cyclopropylmethyl)-11-methyl-8-oxo-2,3,7,12-tetrazatricyclo[7.4.0.0\hat{ }2,6]trideca-1(9),3,5-trien-7-yl]benzamide (Compound 1); 4-((R)-7-(4-bromo-3-(trifluoromethyl)benzoyl)-2-(((S)-but-3-en-2-yl)amino)-6-methyl-4-oxo-5,6,7,8-tetrahydropyrido[3,4-d]pyrimidin-3(4H)-yl)-N-methylbenzamide (Compound 2); N-[4-fluoro-3-(trifluoromethyl)phenyl]-1,3,5-trimethyl-4-[2-oxo-2-[[(1S)-1-(hydroxymethyl)-1-methyl-prop-2-ynyl]amino]acetyl]pyrrole-2-carboxamide (Compound 3); [[2-[5-[(3-cyano-4-fluoro-phenyl)carbamoyl]-1,2,4-trimethyl-pyrrol-3-yl]-2-oxo-acetyl]-(3-ethynyloxetan-3-yl)amino]methyl dihydrogen phosphate (Compound 4); N-(3-cyano-4-fluoro-phenyl)-4-[2-[(3-ethynyloxetan-3-yl)amino]-2-oxo-acetyl]-1,3,5-trimethyl-pyrrole-2-carboxamide (Compound 5); (S)-2-(2-(5-((4-fluoro-3-(trifluoromethyl)phenyl)carbamoyl)-1,2,4-trimethyl-1H-pyrrol-3-yl)-2-oxoacetamido)-2-methylbut-3-yn-1-yl dihydrogen phosphate (Compound 6); (R)-N-(2-chloropyridin-4-yl)-3-fluoro-1-methyl-4-(N-(1,1,1-trifluoropropan-2-yl)sulfamoyl)-1H-pyrrole-2-carboxamide (Compound 7); BAY 414109 (Compound 8); GLS4 (Compound 9); NVR 3-778 (Compound 10); RG7907 (Compound 11); ABI-H0731 (Compound 12); ABI-3773 (Compound 13); ABI-4334 (Compound 14); GLP-26 (Compound 15); KL-060332 (Compound 16); AB-836 (Compound 17); VNRX-9945 (Compound 18); (R)-N-(3-cyano-4-fluorophenyl)-1-methyl-4-(N-(1,1,1-trifluoropropan-2-yl)sulfamoyl)-l H-pyrrole-2-carboxamide (Compound 19); JNJ-64530440 (Compound 20); EDP-514 (Compound 21); (S)-N-(3-cyano-4-fluorophenyl)-7-methyl-3-vinyl-3,4-dihydro-2H,7H-pyrrolo[3,4-b][1,4,5]oxathiazepine-6-carboxamide 1,1-dioxide (Compound 22); ZM-H1505R (Compound 23); (R)-7-(4-bromo-3-chlorobenzoyl)-2-(4-cyclopropoxyphenyl)-6-methyl-3-oxo-N-(2-(pyrimidin-4-yl)benzyl)-2,3,5,6,7,8-hexahydroimidazo[1,5-a]pyrazine-1-carboxamide (Compound 24), (6R)-7-[4-bromo-3-(trifluoromethyl)benzoyl]-2-(3,5-dimethylpyrazol-1-yl)-6-methyl-3-(3-methylimidazo[4,5-b]pyridin-6-yl)-6,8-dihydro-5H-pyrido[3,4-d]pyrimidin-4-one (Compound 25); 4-[5-benzyl-12-(4-bromo-3-chloro-benzoyl)-8-oxo-2,3,7,12-tetrazatricyclo[7.4.0.0\hat{ }2,6]trideca-1(9),3,5-trien-7-yl]-N-methyl-benzamide (Compound 26); and (6R)-7-[4-bromo-3-(trifluoromethyl)benzoyl]-2-(3,5-dimethylpyrazol-1-yl)-3-[4-[(2S)-2-hydroxypropoxy]phenyl]-6-methyl-6,8-dihydro-5H-pyrido[3,4-d]pyrimidin-4-one (Compound 27).

In some embodiments, the HBsAg reducing agent is an siRNA. In some embodiments, the siRNA is a compound selected from the group consisting of RG6346 (Roche/Dicerna), ARC-520 (Arrowhead), ARC-521 (Arrowhead). ALN-HBV (Alnylam/VIR), VIR-2218 (Alnylam/VIR), AB-729 (Arbutus), and JNJ3989 (Arrowhead/JNJ). In some embodiments, the siRNA has a nucleic acid sequence as set forth in SEQ ID NO: 2, SEQ ID NO: 3, SEQ ID NO: 4, SEQ ID NO: 5, SEQ ID NO: 6, or SEQ ID NO: 7. In some embodiments, the HBsAg reducing agent is an ASO. In some embodiments, the ASO is a compound selected from the group consisting of GSK-404 (Isis/GlaxoSmithKline), GSK-836 (Isis/GlaxoSmithKline), and RG6004 (Roche). In some embodiments, the ASO has a nucleic acid sequence as set forth in SEQ ID NO: 1.

In some embodiments, initial administration of the second agent occurs after HBsAg levels have been reduced in the subject by administration of the first agent. In some embodiments, initial administration of the second agent occurs after HBsAg levels have been reduced to a nadir in the subject by administration of the first agent. In some embodiments, the nadir comprises a period of at least one week including at least one additional dose of the first agent, wherein the at least one additional dose results in no statistically significant reduction of HBsAg levels.

In some embodiments, initial administration of the second agent occurs after the first agent has been continuously administered for at least one month. In some embodiments, the delay period is greater than approximately 50 days. In some embodiments, the delay period is greater than approximately 2 months. In some embodiments, the delay period is between approximately 21 days and approximately 168 days. In some embodiments, the delay period is between approximately 28 days and approximately 91 days. In some embodiments, the delay period is between approximately 8 weeks and approximately 18 weeks. In some embodiments, the delay period is at least approximately 70 days. In some embodiments, the delay period is approximately 50 days.

In some embodiments, the first agent is administered at least three times at regular intervals before administration of the second agent. In some embodiments, the second agent is Compound 1, the first agent is an siRNA having the nucleic acid sequences as set forth in SEQ ID NO: 2 and SEQ ID NO: 3, and the delay period includes at least 50 days. In some embodiments, the delay period is at least approximately 70 days.

In some embodiments, the delay period is determined based on measurement of the subject's plasma HBsAg levels. In some embodiments, the delay period extends until the subject's HBsAg levels are reduced as compared to baseline HBsAg levels. In some embodiments, the delay period extends until the subject's plasma HBsAg levels reach a nadir.

Provided herein are methods of maintaining low plasma HBsAg levels in a subject having an HBV infection. In some embodiments, the method incudes administering an effective amount of a first agent selected from the group consisting of (i) an HBV surface antigen (HBsAg) reducing agent, or a pharmaceutically acceptable salt thereof, and (ii) a capsid assembly modulator (CAM), or a pharmaceutically acceptable salt thereof, to the subject, followed by administering an effective amount of a second agent selected from the group consisting of (i) a CAM, or a pharmaceutically acceptable salt thereof, and (ii) an HBsAg reducing agent, or a pharmaceutically acceptable salt thereof, to the subject, wherein initial administration of the second agent is after a delay period following initial administration of the first agent. In some embodiments, when the first agent is an HBsAg reducing agent or a pharmaceutically acceptable salt thereof, the second agent is a CAM or a pharmaceutically acceptable salt thereof. In some embodiments, when the first agent is a CAM or a pharmaceutically acceptable salt thereof, the second agent is an HBsAg reducing agent or a pharmaceutically acceptable salt thereof.

In some embodiments, the first agent is an HBsAg reducing agent or a pharmaceutically acceptable salt thereof; and wherein the second agent is a CAM or a pharmaceutically acceptable salt thereof. In some embodiments, the first agent is a CAM or a pharmaceutically acceptable salt thereof, and wherein the second agent is an HBsAg reducing agent or a pharmaceutically acceptable salt thereof.

In some embodiments, the first agent is a short interfering RNA (siRNA), and the second agent is a Class A capsid assembly modulator (CAM A) or a Class E capsid assembly modulator (CAM-E) or a pharmaceutically acceptable salt thereof. In some embodiments the first agent is a Class A capsid assembly modulator (CAM A) or a Class E capsid assembly modulator (CAM-E) or a pharmaceutically acceptable salt thereof, and the second agent is a short interfering RNA (siRNA).

Provided herein are improved methods of treating a hepatitis B viral infection in a subject for whom therapy with a first agent selected from the group consisting of (i) an HBV surface antigen (HBsAg) reducing agent, or a pharmaceutically acceptable salt thereof, and (ii) a capsid assembly modulator (CAM), or a pharmaceutically acceptable salt thereof, has been initiated. In some embodiments, the improved method includes administering an effective amount of a second agent selected from the group consisting of (i) a CAM, or a pharmaceutically acceptable salt thereof, and (ii) an HBsAg reducing agent, or a pharmaceutically acceptable salt thereof, to the subject. In some embodiments, when the first agent is an HBsAg reducing agent or a pharmaceutically acceptable salt thereof, the second agent is a CAM or a pharmaceutically acceptable salt thereof, and when the first agent is a CAM or a pharmaceutically acceptable salt thereof, the second agent is an HBsAg reducing agent or a pharmaceutically acceptable salt thereof. In some embodiments, the initial administration of the second agent is after a delay period following initial administration of the first agent.

In some embodiments, the first agent is an HBsAg reducing agent or a pharmaceutically acceptable salt thereof; and the second agent is a CAM or a pharmaceutically acceptable salt thereof. In some embodiments, the first agent is a CAM or a pharmaceutically acceptable salt thereof; and the second agent is an HBsAg reducing agent or a pharmaceutically acceptable salt thereof.

In some embodiments, the CAM is a Class A CAM (CAM-A). In some embodiments, the CAM is a Class E CAM (CAM-E). In some embodiments, the HBsAg reducing agent is a small interfering RNA (siRNA) or an antisense oligonucleotide (ASO).

In some embodiments, the CAM is a fused pyrazole compound or a fused pyrimidone compound. In some embodiments, the CAM is selected from the group consisting of: N-methyl-4-[(11R)-12-[4-bromo-3-(trifluoromethyl)benzoyl]-5-(cyclopropylmethyl)-11-methyl-8-oxo-2,3,7,12-tetrazatricyclo[7.4.0.0\hat{ }2,6]trideca-1(9),3,5-trien-7-yl]benzamide (Compound 1); 4-((R)-7-(4-bromo-3-(trifluoromethyl)benzoyl)-2-(((S)-but-3-en-2-yl)amino)-6-methyl-4-oxo-5,6,7,8-tetrahydropyrido[3,4-d]pyrimidin-3(4H)-yl)-N-methylbenzamide (Compound 2); N-[4-fluoro-3-(trifluoromethyl)phenyl]-1,3,5-trimethyl-4-[2-oxo-2-[[(1S)-1-(hydroxymethyl)-1-methyl-prop-2-ynyl]amino]acetyl]pyrrole-2-carboxamide (Compound 3); [[2-[5-[(3-cyano-4-fluoro-phenyl)carbamoyl]-1,2,4-trimethyl-pyrrol-3-yl]-2-oxo-acetyl]-(3-ethynyloxetan-3-yl)amino]methyl dihydrogen phosphate (Compound 4); N-(3-cyano-4-fluoro-phenyl)-4-[2-[(3-ethynyloxetan-3-yl)amino]-2-oxo-acetyl]-1,3,5-trimethyl-pyrrole-2-carboxamide (Compound 5); (S)-2-(2-(5-((4-fluoro-3-(trifluoromethyl)phenyl)carbamoyl)-1,2,4-trimethyl-1H-pyrrol-3-yl)-2-oxoacetamido)-2-methylbut-3-yn-1-yl dihydrogen phosphate (Compound 6); (R)-N-(2-chloropyridin-4-yl)-3-fluoro-1-methyl-4-(N-(1,1,1-trifluoropropan-2-yl)sulfamoyl)-1H-pyrrole-2-carboxamide (Compound 7); BAY 41-4109 (Compound 8); GLS4 (Compound 9); NVR 3-778 (Compound 10); RG7907 (Compound 11); ABI-H0731 (Compound 12); ABI-3773 (Compound 13); ABI-4334 (Compound 14); GLP-26 (Compound 15); KL-060332 (Compound 16); AB-836 (Compound 17); VNRX-9945 (Compound 18); (R)-N-(3-cyano-4-fluorophenyl)-1-methyl-4-(N-(1,1,1-trifluoropropan-2-yl)sulfamoyl)-1H-pyrrole-2-carboxamide (Compound 19); JNJ-64530440 (Compound 20); EDP-514 (Compound 21); (S)-N-(3-cyano-4-fluorophenyl)-7-methyl-3-vinyl-3,4-dihydro-2H,7H-pyrrolo[3,4-b][1,4,5]oxathiazepine-6-carboxamide 1,1-dioxide (Compound 22); ZM-H1505R (Compound 23); (R)-7-(4-bromo-3-chlorobenzoyl)-2-(4-cyclopropoxyphenyl)-6-methyl-3-oxo-N-(2-(pyrimidin-4-yl)benzyl)-2,3,5,6,7,8-hexahydroimidazo[1,5-a]pyrazine-1-carboxamide (Compound 24); (6R)-7-[4-bromo-3-(trifluoromethyl)benzoyl]-2-(3,5-dimethylpyrazol-1-yl)-6-methyl-3-(3-methylimidazo[4,5-b]pyridin-6-yl)-6,8-dihydro-5H-pyrido[3,4-d]pyrimidin-4-one (Compound 25); 4-[5-benzyl-12-(4-bromo-3-chloro-benzoyl)-8-oxo-2,3,7,12-tetrazatricyclo[7.4.0.0\hat{ }2,6]trideca-1(9),3,5-trien-7-yl]-N-methyl-benzamide (Compound 26); and (6R)-7-[4-bromo-3-(trifluoromethyl)benzoyl]-2-(3,5-dimethylpyrazol-1-yl)-3-[4-[(2S)-2-hydroxypropoxy]phenyl]-6-methyl-6,8-dihydro-5H-pyrido[3,4-d]pyrimidin-4-one (Compound 27).

In some embodiments, the HBsAg reducing agent is an siRNA. In some embodiments, the siRNA is a compound selected from the group consisting of RG6346 (Roche/Dicerna), ARC-520 (Arrowhead), ARC-521 (Arrowhead), ALN-HBV (Alnylam/VIR), VIR-2218 (Alnylam/VIR), AB-729 (Arbutus), and JNJ3989 (Arrowhead/JNJ). In some embodiments, the siRNA has a nucleic acid sequence as set forth in SEQ ID NO; 2, SEQ ID NO; 3, SEQ ID NO: 4, SEQ ID NO: 5, SEQ ID NO: 6, or SEQ ID NO: 7. In some embodiments, the HBsAg reducing agent is an ASO. In some embodiments, the ASO is a compound selected from the group consisting of GSK-404 (Isis/GlaxoSmithKline), GSK-836 (Isis/GlaxoSmithKline), and RG6004 (Roche). In some embodiments, the ASO has a nucleic acid sequence as set forth in SEQ ID NO: 1.

In some embodiments, initial administration of the second agent occurs after the first agent has been continuously administered for at least one month. In some embodiments, the delay period is greater than approximately 50 days. In some embodiments, the delay period is greater than approximately 2 months. In some embodiments, the delay period is between approximately 21 days and approximately 168 days. In some embodiments, the delay period is between approximately 28 days and approximately 91 days. In some embodiments, the delay period is between approximately 8 weeks and approximately 18 weeks.

In some embodiments, the first agent is administered at least three times at regular intervals before administration of the second agent. In some embodiments, the CAM is a CAM-A, the CAM-A is Compound 1, the HBsAg reducing agent is an siRNA having the nucleic acid sequences set forth in SEQ ID NO: 2 and SEQ ID NO: 3, and the delay period includes at least approximately 50 days. In some embodiments, the delay period is at least approximately 70 days. In some embodiments, the first agent is Compound 1 or a pharmaceutically acceptable salt thereof, and the second agent is an siRNA having the nucleic acid sequences set forth in SEQ ID NO: 2 and SEQ ID NO: 3. In some embodiments, the first agent is an siRNA having the nucleic acid sequences set forth in SEQ ID NO: 2 and SEQ ID NO: 3, and the second agent is Compound 1 or a pharmaceutically acceptable salt thereof. In some embodiments, the hepatitis B viral infection is a chronic hepatitis B viral infection.

In some embodiments, (a) the first agent is Compound 4 or pharmaceutically acceptable salt thereof and the second agent is an siRNA having the nucleic acid sequences set forth in SEQ ID NO: 6 and SEQ ID NO: 7, or (b) the first agent is an siRNA having the nucleic acid sequences set forth in SEQ ID NO: 6 and SEQ ID NO: 7, and the second agent is Compound 4 or a pharmaceutically acceptable salt thereof.

In some embodiments, the method further comprises administering an additional agent selected from the group consisting of an interferon, a nucleoside analog, a nucleotide analog, a sequence specific oligonucleotide, a nucleic acid polymer, an entry inhibitor and a small molecule immunomodulator. In some embodiments, the additional agent is selected from the group consisting of recombinant interferon alpha 2b, IFN-α, PEG-IFN-α-2a, lamivudine, telbivudine, adefovir dipivoxil, clevudine, entecavir, tenofovir alafenamide, and tenofovir disoproxil and an additional siRNA.

### BRIEF DESCRIPTION OF THE DRAWINGS

FIG. 1 illustrates a dosing schedule used in the experiment of Example 1.

FIG. 2A illustrates a graph showing HBV DNA measurements over time in the experiment of Example 1.

FIG. 2B illustrates a graph showing HBV DNA measurements at day 189 in the experiment of Example 1.

FIG. 3A illustrates a graph showing HBsAg measurements over time in the experiment of Example 1.

FIG. 3B illustrates a graph showing HBsAg measurements at day 189 in the experiment of Example 1.

FIG. 4A illustrates a graph showing HBeAg measurements over time in the experiment of Example 1.

FIG. 4B illustrates a graph showing HBeAg measurements at day 189 in the experiment of Example 1.

FIG. 5 illustrates a dosing schedule used in the experiment of Example 2.

FIG. 6A illustrates a graph showing serum HBsAg measurements over time in the experiment of Example 2.

FIG. 6B illustrates a graph showing serum HBsAg measurements over time including add-on groups in the experiment of Example 2.

FIG. 7A illustrates a graph showing serum HBeAg measurements over time in the experiment of Example 2.

FIG. 7B illustrates a graph showing serum HBeAg measurements over time including add-on groups in the experiment of Example 2.

FIG. 8A illustrates a graph showing serum HBV DNA measurements over time in the experiment of Example 2.

FIG. 8B illustrates a graph showing serum HBV DNA measurements over time including add-on groups in the experiment of Example 2.

FIG. 9 illustrates a dosing schedule used in the experiment of Example 2.

FIG. 10A illustrates a graph showing serum HBsAg measurements over time in the experiment of Example 2.

FIG. 10B illustrates a graph showing serum HBsAg measurements over time including add-on groups in the experiment of Example 2.

FIG. 11A illustrates a graph showing serum HBeAg measurements over time in the experiment of Example 2.

FIG. 11B illustrates a graph showing serum HBeAg measurements over time including add-on groups in the experiment of Example 2.

FIG. 12A illustrates a graph showing serum HBV DNA measurements over time in the experiment of Example 2.

FIG. 12B illustrates a graph showing serum HBV DNA measurements over time including add-on groups in the experiment of Example 2.

FIG. 13A illustrates a graph showing HBV DNA measurements over time in the experiment of Example 3.

FIG. 13B illustrates a graph showing HBV DNA measurements at day 98 in the experiment of Example 3.

FIG. 14A illustrates a graph showing HBsAg measurements over time in the experiment of Example 3.

FIG. 14B illustrates a graph showing HBsAg measurements at day 98 in the experiment of Example 3.

FIG. 15A illustrates a graph showing HBeAg measurements over time in the experiment of Example 3.

FIG. 15B illustrates a graph showing HBeAg measurements at day 98 in the experiment of Example 3.

FIG. 16A illustrates a graph showing HBV DNA measurements over time in the experiment of Example 3.

FIG. 16B illustrates a graph showing HBV DNA measurements at day 98 in the experiment of Example 3.

FIG. 17A illustrates a graph showing HBsAg measurements over time in the experiment of Example 3.

FIG. 17B illustrates a graph showing HBsAg measurements at day 98 in the experiment of Example 3.

FIG. 18A illustrates a graph showing HBeAg measurements over time in the experiment of Example 3.

FIG. 18B illustrates a graph showing HBeAg measurements at day 98 in the experiment of Example 3.

### DETAILED DESCRIPTION

HBV has a partially double-stranded circular DNA genome of about 3.2 kilobase (kb) pairs, and is classified into at least eight genotypes. The HBV replication pathway has been studied in great detail. See Tsukuda and Watashi, Hepatitis B Virus Biology and Life Cycle, Antiviral Res. 2020 October;182:104925. Part of the replication cycle includes the formation of the covalently closed circular (cccDNA) form. The presence of the cccDNA gives rise to the risk of viral reemergence throughout the life of the host organism. HBV carriers can transmit the disease for many years. An estimated 300 million people are living with chronic hepatitis B, and it is estimated that over 750,000 people worldwide die of hepatitis B each year. In addition, immunosuppressed individuals or individuals undergoing chemotherapy are especially at risk for reactivation of HBV infection. Hepatitis B can be acute or chronic. Acute HBV infection can be either asymptomatic or present as symptomatic acute hepatitis.

HBV can be transmitted by blood, semen, and/or another body fluid. This can occur through direct blood-to-blood contact, unprotected sex, sharing of needles, but mostly from an infected mother to her baby during the delivery process. The HBV surface antigen (HBsAg) is most frequently used to screen for the presence of this infection. Currently available medications rarely cure HBV and/or HDV infection. Rather, the medications suppress replication of the virus.

A capsid assembly modulator (CAM) blocks the encapsidation of HBV pregenomic RNA (pgRNA) into viral particles and subsequent reverse transcription to relaxed circular DNA (rcDNA) by speeding up the assembly of the HBV core protein (HBc). Most CAMs also block the establishment of cccDNA, the main reservoir for HBV. CAMs can be grouped into two classes. Class A molecules, formerly known as Class I molecules (CAM-1), including heteroaryldihydropyrimidine (HAP) compounds, induce the formation of large aggregates of core proteins. Class E (Class E) molecules, formerly known as Class II molecules (CAM-2), including phenylpropenamides (PPAs) and sulfamoylbenzamides (SBAs), produce empty capsids that are devoid of pgRNA.

siRNA therapy for treating HBV is described, for example, in Chen and Mahato, “siRNA Pool Targeting Different Sites of Human Hepatitis B Surface Antigen Efficiently Inhibits HBV Infection;” J Drug Target. 2008 February; 16(2): 140-148 and Morrissey et al., “Potent and persistent in vivo anti-HBV activity of chemically modified siRNAs,” Nature Biotechnology 23, 1002-1007 (2005). RNAi is a sequence-specific, post-transcriptional gene silencing mechanism, which is triggered by double-stranded synthetic siRNA or short hairpin RNA (shRNA) expressed intracellularly from a vector. HBV replication and expression can be inhibited by administration of synthetic siRNAs or endogenously expressed shRNAs. See, for example, Giladi et al., “Small interfering RNA inhibits hepatitis B virus replication in mice,” Mol Ther. 2003; 8(5):769-76; McCaffrey et al., “Inhibition of hepatitis B virus in mice by RNA interference,” Nat Biotechnol. 2003; 21(6):639-44; and Shlomai and Shaul, “Inhibition of hepatitis B virus expression and replication by RNA interference,” Hepatology. 2003; 37(4):764-70). HBV gene silencing may depend, for example, on siRNA dosing and sequences, and targets for gene silencing include, for example, the inhibition of virus replication, and suppression of HBsAg expression.

Antisense oligonucleotide therapy for treating HBV is described, for example, in Korba and Gerin, “Antisense oligonucleotides are effective inhibitors of hepatitis B virus replication in vitro,” Antiviral Res. 1995 Nov; 28(3): 225-42. Antisense oligonucleotides directed against the HBsAg gene can suppress virus production. Antisense oligonucleotides (ASOs) are effective in reducing HBsAg in animal models, and in CHB patients.

Therapies for treating HBV infection may be combined to further advantageous effect. For example, administration of a first agent such as an HBsAg reducing agent, for example, an siRNA and/or an ASO, or a CAM (Class A or Class E), may be combined with administration of a second agent of a different type, for example, a CAM (Class A or Class E) where the first agent is an HBsAg reducing agent, or an HBsAg reducing agent, for example, an siRNA and/or an ASO, where the first agent is a CAM.

Surprisingly and unexpectedly, delaying administration of the second agent for a period of time after the initial administration of the first agent (for example, providing an add-on of a CAM to continuous siRNA/ASO treatment, or providing an add-on of siRNA/ASO to a continuous CAM treatment), as opposed to starting both treatments simultaneously, provides improved results in reducing HBV infection in a subject, and in avoiding antagonistic effects observed when both agents are administered from the beginning of treatment.

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the drawings, can be arranged, substituted, combined, separated, and designed in a wide variety of different configurations, all of which are explicitly contemplated herein.

It is to be understood that this disclosure is not limited to particular embodiments described, as such may, of course, vary. It is also to be understood that the terminology used herein is for the purpose of describing particular embodiments only, and is not intended to be limiting.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

All patents, applications, published applications and other publications referenced herein are incorporated by reference in their entirety unless stated otherwise. For purposes of the present disclosure, the following terms are defined below.

### Definitions

Unless defined otherwise, technical and scientific terms used herein have the same meaning as commonly understood by one of ordinary skill in the art to which the present disclosure belongs. In the event that there are a plurality of definitions for a term herein, those in this section prevail unless stated otherwise.

The terms “treating,” “treatment,” “therapeutic,” or “therapy” as used herein has its ordinary meaning as understood in light of the specification, and do not necessarily mean total cure or abolition of the disease or condition. The term “treating” or “treatment” as used herein (and as well understood in the art) also means an approach for obtaining beneficial or desired results in a subject's condition, including clinical results. Beneficial or desired clinical results can include, but are not limited to, alleviation or amelioration of one or more symptoms or conditions, diminishment of the extent of a disease, stabilizing (i.e., not worsening) the state of disease, prevention of a disease's transmission or spread, delaying or slowing of disease progression, amelioration or palliation of the disease state, diminishment of the reoccurrence of disease, and remission, whether partial or total and whether detectable or undetectable. “Treating” and “treatment” as used herein also include prophylactic treatment. Treatment methods comprise administering to a subject a therapeutically effective amount of an active agent. The administering step may consist of a single administration or may comprise a series of administrations. The compositions are administered to the subject in an amount and for a duration sufficient to treat the subject. The length of the treatment period depends on a variety of factors, such as the severity of the condition, the age and genetic profile of the subject, the concentration of active agent, the activity of the compositions used in the treatment, or a combination thereof. It will also be appreciated that the effective dosage of an agent used for the treatment or prophylaxis may increase or decrease over the course of a particular treatment or prophylaxis regime. Changes in dosage may result and become apparent by standard diagnostic assays known in the art. In some instances, chronic administration may be required.

The term “effective amount” is used to indicate an amount of an active compound, or pharmaceutical agent, that elicits the biological or medicinal response indicated. For example, an effective amount of compound can be the amount needed to alleviate or ameliorate symptoms of disease or prolong the survival of the subject being treated. This response may occur in a tissue, system, animal or human and includes alleviation of the signs or symptoms of the disease being treated. Determination of an effective amount is well within the capability of those skilled in the art, in view of the disclosure provided herein. The effective amount of the compounds disclosed herein required as a dose will depend on the route of administration, the type of animal, including human, being treated, and the physical characteristics of the specific animal under consideration. The dose can be tailored to achieve a desired effect, but will depend on such factors as weight, diet, concurrent medication and other factors which those skilled in the medical arts will recognize.

A therapeutically effective amount can also be an amount of the compound sufficient to reduce HBsAg levels consistent with evolution to clinical seroconversion; achieve sustained HBsAg clearance associated with reduction of infected hepatocytes by a subject's immune system; induce HBV-antigen specific activated T-cell populations; and/or achieve persistent loss of HBsAg within 12 months. Examples of a target index include lower HBsAg below a threshold of 500 HBsAg international units (IU) and/or higher CD8 counts. Additional examples of target indexes include, but are not limited to, serum HBV DNA levels lower than the lower limit of quantification (LLoQ) or lower than 20 IU/mL, more particularly lower than 15 IU/mL, more particularly lower than 10 IU/mL; serum ALT concentration lower than 3 times the upper normal limit, or lower than 129 U/L if the subject is a male subject, or lower than 108 U/L if the subject is a female subject, more particularly a serum ALT concentration lower than 120 U/L if the subject is a male subject or lower than 105 U/L if the subject is a female subject, more particularly a serum ALT concentration lower than 90 U/L if the subject is a male subject or lower than 57 U/L if the subject is a female subject; HBsAg-negative serum; serum HBsAg level of 100 IU/mL or lower, more particularly of 10 IU/mL or lower; and/or HBs seroconversion.

As used herein, the term “capsid assembly modulator” or “CAM” refers to a compound that disrupts or accelerates or inhibits or hinders or delays or reduces or modifies normal capsid assembly (such as during maturation) or normal capsid disassembly (such as during infectivity) or perturbs capsid stability, thereby inducing aberrant capsid morphology and function. In some embodiments, a capsid assembly modulator accelerates capsid assembly or disassembly, thereby inducing aberrant capsid morphology. In another embodiment, a capsid assembly modulator interacts (for instance, binds at an active site, binds at an allosteric site, modifies or hinders folding and the like) with the core protein, thereby disrupting capsid assembly or disassembly. In yet another embodiment, a capsid assembly modulator causes a perturbation in structure or function of core protein (such as the ability of core protein to assemble, disassemble, bind to a substrate, fold into a suitable conformation, or the like), which attenuates viral infectivity or is lethal to the virus.

As used herein, the term “fused pyrazole compound” refers to a chemical compound with a pyrazole ring fused to another ring.

As used herein, the term “fused pyrimidone compound” refers to a chemical compound with a pyrimidone ring fused to another ring.

Whenever a group is described as being “optionally substituted” that group may be unsubstituted or substituted with one or more of the indicated substituents. Likewise, when a group is described as being “unsubstituted or substituted” if substituted, the substituent(s) may be selected from one or more of the indicated substituents. If no substituents are indicated, it is meant that the indicated “optionally substituted” or “substituted” group may be substituted with one or more group(s) (such as 1, 2 or 3 groups) individually and independently selected from deuterium, alkyl, alkenyl, alkynyl, cycloalkyl, cycloalkenyl, aryl, heteroaryl, heterocyclyl, aryl(alkyl), heteroaryl(alkyl), (heterocyclyl)alkyl, hydroxy, alkoxy, acyl, cyano, halogen, thiocarbonyl, O-carbamyl, N carbamyl, O thiocarbamyl, N thiocarbamyl, C amido, N amido, S-sulfonamido, N sulfonamido, C carboxy, O carboxy, isocyanato, thiocyanato, isothiocyanato, nitro, azido, silyl, sulfenyl, sulfinyl, sulfonyl, haloalkyl, haloalkoxy, trihalomethanesulfonyl, trihalomethanesulfonamido, an amino, a mono substituted amine and a di substituted amine.

As used herein, “Cₐ to C_{b}” or “C_{a-b}” in which “a” and “b” are integers refer to the number of carbon atoms in an alkyl, alkenyl or alkynyl group, or the number of carbon atoms in the ring of a cycloalkyl, cycloalkenyl, aryl, heteroaryl or heterocyclyl group. That is, the alkyl, alkenyl, alkynyl, ring of the cycloalkyl, ring of the cycloalkenyl, ring of the aryl, ring of the heteroaryl or ring of the heterocyclyl can contain from “a” to “b”, inclusive, carbon atoms. Thus, for example, a “C₁ to C₄ alkyl” or “C₁₋₄ alkyl” group refers to all alkyl groups having from 1 to 4 carbons, that is, CH₃—, CH₃CH₂—, CH₃CH₂CH₂—, (CH₃)₂CH—, CH₃CH₂CH₂CH₂—, CH₃CH₂CH(CH₃)— and (CH₃)₃C—. If no “a” and “b” are designated with regard to an alkyl, alkenyl, alkynyl, cycloalkyl cycloalkenyl, aryl, heteroaryl or heterocyclyl group, the broadest range described in these definitions is to be assumed.

As used herein, “alkyl” refers to a straight or branched hydrocarbon chain that comprises a fully saturated (no double or triple bonds) hydrocarbon group. The alkyl group may have 1 to 20 carbon atoms (whenever it appears herein, a numerical range such as “1 to 20” refers to each integer in the given range; for example, “1 to 20 carbon atoms” means that the alkyl group may consist of 1 carbon atom, 2 carbon atoms, 3 carbon atoms, etc., up to and including 20 carbon atoms, although the present definition also covers the occurrence of the term “alkyl” where no numerical range is designated). The alkyl group may also be a medium size alkyl having 1 to 10 carbon atoms. The alkyl group could also be a lower alkyl having 1 to 6 carbon atoms. The alkyl group of the compounds may be designated as “C₁-C₄ alkyl” or similar designations. By way of example only, “C₁-C₄ alkyl” indicates that there are one to four carbon atoms in the alkyl chain, i.e., the alkyl chain is selected from methyl, ethyl, propyl, iso-propyl, n-butyl, iso-butyl, sec-butyl and t-butyl. Typical alkyl groups include, but are in no way limited to, methyl, ethyl, propyl, isopropyl, butyl, isobutyl, tertiary butyl, pentyl and hexyl. The alkyl group may be substituted or unsubstituted.

As used herein, “alkenyl” refers to an alkyl group that contains in the straight or branched hydrocarbon chain one or more double bonds. The length of an alkenyl can vary. For example, the alkenyl can be a C₂₋₄ alkenyl, C₂₋₆ alkenyl or C₂₋₈ alkenyl. Examples of alkenyl groups include allenyl, vinylmethyl and ethenyl. An alkenyl group may be unsubstituted or substituted.

As used herein, “alkynyl” refers to an alkyl group that contains in the straight or branched hydrocarbon chain one or more triple bonds. The length of an alkynyl can vary. For example, the alkynyl can be a C₂₋₄ alkynyl, C₂₋₆ alkynyl or C₂₋₈ alkynyl. Examples of alkynyls include ethynyl and propynyl. An alkynyl group may be unsubstituted or substituted.

As used herein, “cycloalkyl” refers to a completely saturated (no double or triple bonds) mono- or multi-cyclic hydrocarbon ring system. When composed of two or more rings, the rings may be joined together in a fused fashion. Cycloalkyl groups can contain 3 to 10 atoms in the ring(s). 3 to 8 atoms in the ring(s) or 3 to 6 atoms in the ring(s). A cycloalkyl group may be unsubstituted or substituted. Typical cycloalkyl groups include, but are in no way limited to, cyclopropyl, cyclobutyl, cyclopentyl, cyclohexyl, cycloheptyl and cyclooctyl.

As used herein, “cycloalkenyl” refers to a mono- or multi-cyclic hydrocarbon ring system that contains one or more double bonds in at least one ring; although, if there is more than one, the double bonds cannot form a fully delocalized pi-electron system throughout all the rings (otherwise the group would be “aryl,” as defined herein). When composed of two or more rings, the rings may be connected together in a fused fashion. A cycloalkenyl can contain 3 to 10 atoms in the ring(s) or 3 to 8 atoms in the ring(s). A cycloalkenyl group may be unsubstituted or substituted.

As used herein, “aryl” refers to a carbocyclic (all carbon) monocyclic or multicyclic aromatic ring system (including fused ring systems where two carbocyclic rings share a chemical bond) that has a fully delocalized pi-electron system throughout all the rings. The number of carbon atoms in an aryl group can vary. For example, the aryl group can be a C₆-C₁₄ aryl group, a C₆-C₁₀ aryl group, or a C aryl group. Examples of aryl groups include, but are not limited to, benzene, naphthalene and azulene. An aryl group may be substituted or unsubstituted.

As used herein, “heteroaryl” refers to a monocyclic, bicyclic and tricyclic aromatic ring system (a ring system with fully delocalized pi-electron system) that contain(s) one or more heteroatoms (for example, 1 to 5 heteroatoms), that is, an element other than carbon, including but not limited to, nitrogen, oxygen and sulfur. The number of atoms in the ring(s) of a heteroaryl group can vary. For example, the heteroaryl group can contain 4 to 14 atoms in the ring(s), 5 to 10 atoms in the ring(s) or 5 to 6 atoms in the ring(s). Furthermore, the term “heteroaryl” includes fused ring systems where two rings, such as at least one aryl ring and at least one heteroaryl ring, or at least two heteroaryl rings, share at least one chemical bond. Examples of heteroaryl rings include, but are not limited to, furan, furazan, thiophene, benzothiophene, phthalazine, pyrrole, oxazole, benzoxazole, 1,2,3-oxadiazole, 1,2,4-oxadiazole, thiazole, 1,2,3-thiadiazole, 1,2,4-thiadiazole, benzothiazole, imidazole, benzimidazole, indole, indazole, pyrazole (including, for example, an optionally substituted pyrazol-1-yl), benzopyrazole, isoxazole, benzoisoxazole, isothiazole, triazole, benzotriazole, thiadiazole, tetrazole, pyridine, pyridazine, pyrimidine, pyrazine, purine, pteridine, quinoline, isoquinoline, quinazoline, quinoxaline, cinnoline and triazine. A heteroaryl group may be substituted or unsubstituted.

As used herein, “heterocyclyl” refers to a monocyclic, bicyclic and tricyclic ring system wherein carbon atoms together with from 1 to 5 heteroatoms constitute said ring system. A heterocycle may optionally contain one or more unsaturated bonds situated in such a way, however, that a fully delocalized pi-electron system does not occur throughout all the rings. The number of atoms in the ring(s) of a heterocyclyl group can vary. For example, the heterocyclyl group can contain 4 to 14 atoms in the ring(s), 5 to 10 atoms in the ring(s) or 5 to 6 atoms in the ring(s). The heteroatom(s) is an element other than carbon including, but not limited to, oxygen, sulfur and nitrogen. A heterocycle may further contain one or more carbonyl or thiocarbonyl functionalities, so as to make the definition include oxo-systems and thio-systems such as lactams, lactones, cyclic imides, cyclic thioimides and cyclic carbamates. When composed of two or more rings, the rings may be joined together in a fused fashion. Additionally, any nitrogens in a heterocyclyl may be quaternized. Heterocyclyl groups may be unsubstituted or substituted. Examples of such “heterocyclyl groups include but are not limited to, 1,3-dioxin, 1,3-dioxane, 1,4-dioxane, 1,2-dioxolane, 1,3-dioxolane, 1,4-dioxolane, 1,3-oxathiane, 1,4-oxathiin, 1,3-oxathiolane, 1,3-dithiole, 1,3-dithiolane, 1,4-oxathiane, tetrahydro-1,4-thiazine, 2H-1,2-oxazine, maleimide, succinimide, barbituric acid, thiobarbituric acid, dioxopiperazine, hydantoin, dihydrouracil, trioxane, hexahydro-1,3,5-triazine, imidazoline, imidazolidine, isoxazoline, isoxazolidine, oxazoline, oxazolidine, oxazolidinone, thiazoline, thiazolidine, morpholine, oxirane, piperidine N-Oxide, piperidine, piperazine, pyrrolidine, pyrrolidone, pyrrolidione, 4-piperidone, pyrazoline, pyrazolidine, 2-oxopyrrolidine, tetrahydropyran, 4H-pyran, tetrahydrothiopyran, thiamorpholine, thiamorpholine sulfoxide, thiamorpholine sulfone and their benzo-fused analogs (for example, benzimidazolidinone, tetrahydroquinoline and 3,4-methylenedioxyphenyl).

As used herein, “aryl(alkyl)” refers to an aryl group connected, as a substituent, via a lower alkylene group. The lower alkylene and aryl group of an aryl(alkyl) may be substituted or unsubstituted. Examples include but are not limited to benzyl, 2-phenyl(alkyl), 3-phenyl(alkyl), and naphthyl(alkyl).

As used herein, “heteroaryl(alkyl)” refers to a heteroaryl group connected, as a substituent, via a lower alkylene group. The lower alkylene and heteroaryl group of heteroaryl(alkyl) may be substituted or unsubstituted. Examples include but are not limited to 2-thienyl(alkyl), 3-thienyl(alkyl), furyl(alkyl), thienyl(alkyl), pyrrolyl(alkyl), pyridyl(alkyl), isoxazolyl(alkyl), imidazolyl(alkyl) and their benzo-fused analogs.

A “(heterocyclyl)alkyl” refers to a heterocyclic group connected, as a substituent, via a lower alkylene group. The lower alkylene and heterocyclyl of a heterocyclyl(alkyl) may be substituted or unsubstituted. Examples include but are not limited tetrahydro-2H-pyran-4-yl(methyl), piperidin-4-yl(ethyl), piperidin-4-yl(propyl), tetrahydro-2H-thiopyran-4-yl(methyl) and 1,3-thiazinan-4-yl(methyl).

“Lower alkylene groups” are straight-chained —CH₂— tethering groups, forming bonds to connect molecular fragments via their terminal carbon atoms. In some embodiments, a lower alkylene can include 1, 2, 3, 4, 5 or 6 carbons. Examples include but are not limited to methylene (—CH₂—), ethylene (—CH₂CH₂—), propylene (—CH₂CH₂CH₂—) and butylene (—CH₂CH₂CH₂CH₂—). A lower alkylene group can be substituted by replacing one or more hydrogen of the lower alkylene group with a substituent(s) listed under the definition of “substituted” and/or by substituting both hydrogens on the same carbon with a cycloalkyl group (for example,

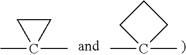

or a monocyclic heterocyclyl (such as

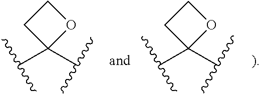 [SMILES: *C1(*)CCO1 ; *C1(*)CCO1]

As used herein, “alkoxy” refers to the formula —OR wherein R is an alkyl, an alkenyl, an alkynyl, a cycloalkyl, a cycloalkenyl, aryl, heteroaryl, heterocyclyl, aryl(alkyl), heteroaryl(alkyl) or heterocyclyl(alkyl) is defined herein. A non-limiting list of alkoxys are methoxy, ethoxy, n-propoxy, 1-methylethoxy (isopropoxy), n-butoxy, iso-butoxy, sec-butoxy, tert-butoxy, phenoxy and benzoxy. In some instances, an alkoxy can be —OR wherein R is an unsubstituted C₁₋₄ alkyl. An alkoxy may be substituted or unsubstituted.

As used herein, “acyl” refers to a hydrogen an alkyl, an alkenyl, an alkynyl, a cycloalkyl, a cycloalkenyl, aryl, heteroaryl, heterocyclyl, aryl(alkyl), heteroaryl(alkyl) or heterocyclyl(alkyl) connected, as substituents, via a carbonyl group. Examples include formyl, acetyl, propanoyl, benzoyl, and acryl. An acyl may be substituted or unsubstituted.

As used herein, “hydroxyalkyl” refers to an alkyl group in which one or more of the hydrogen atoms are replaced by a hydroxy group. Exemplary hydroxyalkyl groups include but are not limited to, 2-hydroxyethyl, 3-hydroxypropyl, 2-hydroxypropyl and 2,2-dihydroxyethyl. A hydroxyalkyl may be substituted or unsubstituted.

As used herein, “haloalkyl” refers to an alkyl group in which one or more of the hydrogen atoms are replaced by a halogen (for example, mono-haloalkyl, di-haloalkyl and tri-haloalkyl). Such groups include but are not limited to, chloromethyl, fluoromethyl, difluoromethyl, trifluoromethyl, 1-chloro-2-fluoromethyl and 2-fluoroisobutyl. A haloalkyl may be substituted or unsubstituted.

As used herein, “haloalkoxy” refers to a O-alkyl group in which one or more of the hydrogen atoms are replaced by a halogen (for example, mono-haloalkoxy, di-haloalkoxy and tri-haloalkoxy). Such groups include but are not limited to, chloromethoxy, fluoromethoxy, difluoromethoxy, trifluoromethoxy, 1-chloro-2-fluoromethoxy and 2-fluoroisobutoxy. In some instances, a haloalkoxy can be —OR, wherein R is a C₁₋₄ alkyl substituted by 1, 2 or 3 halogens. A haloalkoxy may be substituted or unsubstituted.

A “sulfenyl” group refers to an “—SR” group in which R can be hydrogen, alkyl, alkenyl, alkynyl, cycloalkyl, cycloalkenyl, aryl, heteroaryl, heterocyclyl, aryl(alkyl), heteroaryl(alkyl) or heterocyclyl(alkyl). A sulfenyl may be substituted or unsubstituted.

A “sulfinyl” group refers to an “—S(═O)R” group in which R can be the same as defined with respect to sulfenyl. A sulfinyl may be substituted or unsubstituted.

A “sulfonyl” group refers to an “—S(═O)₂R” group in which R can be the same as defined with respect to sulfenyl. A sulfonyl may be substituted or unsubstituted.

An “O carboxy” group refers to a “RC(═O)O—” group in which R can be hydrogen, alkyl, alkenyl, alkynyl, cycloalkyl, cycloalkenyl, aryl, heteroaryl, heterocyclyl, aryl(alkyl), heteroaryl(alkyl) or heterocyclyl(alkyl), as defined herein. An O-carboxy may be substituted or unsubstituted.

The terms “ester” and “C carboxy” refer to a “—C(═O)OR” group in which R can be the same as defined with respect to O-carboxy. An ester and C-carboxy may be substituted or unsubstituted.

A “thiocarbonyl” group refers to a “—C(═S)R” group in which R can be the same as defined with respect to O-carboxy. A thiocarbonyl may be substituted or unsubstituted.

A “trihalomethanesulfonyl” group refers to an “X₃CS(═O)₂—” group wherein each X is a halogen.

A “trihalomethanesulfonamido” group refers to an “X₃CS(═O)₂N(R^{A})—” group wherein each X is a halogen, and R^{A} is hydrogen, alkyl, alkenyl, alkynyl, cycloalkyl, cycloalkenyl, aryl, heteroaryl, heterocyclyl, aryl(alkyl), heteroaryl(alkyl) or heterocyclyl(alkyl).

The term “amino” as used herein refers to a —NH₂ group.

As used herein, the term “hydroxy” refers to a —OH group.

A “cyano” group refers to a “—CN” group.

The term “azido” as used herein refers to a —N₃ group.

An “isocyanato” group refers to a “—NCO” group.

A “thiocyanato” group refers to a “—SCN” group.

An “isothiocyanato” group refers to an “—NCS” group.

A “mercapto” group refers to an “—SH” group.

A “carbonyl” group refers to a —C(═O)— group.

An “S sulfonamido” group refers to a “—S(═O)₂N(R^{A}R^{B})” group in which R^{A} and R^{B} can be independently hydrogen, alkyl, alkenyl, alkynyl, cycloalkyl, cycloalkenyl, aryl, heteroaryl, heterocyclyl, aryl(alkyl), heteroaryl(alkyl) or heterocyclyl(alkyl). An S sulfonamido may be substituted or unsubstituted.

An “N sulfonamido” group refers to a “RS(═O)₂N(R^{A})—” group in which R and R^{A} can be independently hydrogen, alkyl, alkenyl, alkynyl, cycloalkyl, cycloalkenyl, aryl, heteroaryl, heterocyclyl, aryl(alkyl), heteroaryl(alkyl) or heterocyclyl(alkyl). An N sulfonamido may be substituted or unsubstituted.

An “O carbamyl” group refers to a “—OC(═O)N(R^{A}R^{B})” group in which R^{A} and R^{B} can be independently hydrogen, alkyl, alkenyl, alkynyl, cycloalkyl, cycloalkenyl, aryl, heteroaryl, heterocyclyl, aryl(alkyl), heteroaryl(alkyl) or heterocyclyl(alkyl). An O carbamyl may be substituted or unsubstituted.

An “N carbamyl” group refers to an “ROC(═O)N(R^{A})—” group in which R and R^{A} can be independently hydrogen, alkyl, alkenyl, alkynyl, cycloalkyl, cycloalkenyl, aryl, heteroaryl, heterocyclyl, aryl(alkyl), heteroaryl(alkyl) or heterocyclyl(alkyl). An N carbamyl may be substituted or unsubstituted.

An “O thiocarbamyl” group refers to a “—OC(═S) N(R^{A}R^{B})” group in which R^{A} and R^{B} can be independently hydrogen, alkyl, alkenyl, alkynyl, cycloalkyl, cycloalkenyl, aryl, heteroaryl, heterocyclyl, aryl(alkyl), heteroaryl(alkyl) or heterocyclyl(alkyl). An O thiocarbamyl may be substituted or unsubstituted.

An “N thiocarbamyl” group refers to an “ROC(═S)N(R^{A})—” group in which R and R^{A} can be independently hydrogen, alkyl, alkenyl, alkynyl, cycloalkyl, cycloalkenyl, aryl, heteroaryl, heterocyclyl, aryl(alkyl), heteroaryl(alkyl) or heterocyclyl(alkyl). An N thiocarbamyl may be substituted or unsubstituted.

A “C amido” group refers to a “—C(═O)N(R^{A}R^{B})” group in which R^{A} and R^{B} can be independently hydrogen, alkyl, alkenyl, alkynyl, cycloalkyl, cycloalkenyl, aryl, heteroaryl, heterocyclyl, aryl(alkyl), heteroaryl(alkyl) or heterocyclyl(alkyl). A C amido may be substituted or unsubstituted.

An “N amido” group refers to a “RC(═O)N(R^{A})—” group in which R and R^{A} can be independently hydrogen, alkyl, alkenyl, alkynyl, cycloalkyl, cycloalkenyl, aryl, heteroaryl, heterocyclyl, aryl(alkyl), heteroaryl(alkyl) or heterocyclyl(alkyl). An N amido may be substituted or unsubstituted.

A “mono substituted amine” refers to a “—NHR^{A}” in which R^{A} can be independently alkyl, alkenyl, alkynyl, cycloalkyl, cycloalkenyl, aryl, heteroaryl, heterocyclyl, aryl(alkyl), heteroaryl(alkyl) or heterocyclyl(alkyl). A mono substituted amine may be substituted or unsubstituted. In some instances, a mono substituted amine can be —NHR^{A}, wherein R^{A} can be an unsubstituted C₁₋₆ alkyl or an unsubstituted or a substituted benzyl.

A “di substituted amine” refers to a “—NR^{A}R^{B}” in which R^{A} and R^{B} can be independently can be independently alkyl, alkenyl, alkynyl, cycloalkyl, cycloalkenyl, aryl, heteroaryl, heterocyclyl, aryl(alkyl), heteroaryl(alkyl) or heterocyclyl(alkyl). A di substituted amine may be substituted or unsubstituted. In some instances, a di substituted amine can be —NR^{A}R^{B}, wherein R^{A} and R^{B} can be independently an unsubstituted C₁₋₆ alkyl or an unsubstituted or a substituted benzyl.

The term “halogen atom” or “halogen” as used herein, means any one of the radio-stable atoms of column 7 of the Periodic Table of the Elements, such as, fluorine, chlorine, bromine and iodine.

Where the numbers of substituents is not specified (for example haloalkyl), there may be one or more substituents present. For example “haloalkyl” may include one or more of the same or different halogens. As another example, “C₁-C₃ alkoxyphenyl” may include one or more of the same or different alkoxy groups containing one, two or three atoms.

As used herein, the abbreviations for any protective groups, amino acids and other compounds, are, unless indicated otherwise, in accord with their common usage, recognized abbreviations, or the IUPAC-IUB Commission on Biochemical Nomenclature (See, Biochem. 11:942-944 (1972)).

The term “pharmaceutically acceptable salt” refers to a salt of a compound that does not cause significant irritation to an organism to which it is administered and does not abrogate the biological activity and properties of the compound. In some embodiments, the salt is an acid addition salt of the compound. Pharmaceutical salts can be obtained by reacting a compound with inorganic acids such as hydrohalic acid (for example, hydrochloric acid or hydrobromic acid), sulfuric acid, nitric acid and phosphoric acid. Pharmaceutical salts can also be obtained by reacting a compound with an organic acid such as aliphatic or aromatic carboxylic or sulfonic acids, for example formic, acetic, succinic, lactic, malic, tartaric, citric, ascorbic, nicotinic, methanesulfonic, ethanesulfonic, p-toluenesulfonic, salicylic or naphthalenesulfonic acid. Pharmaceutical salts can also be obtained by reacting a compound with a base to form a salt such as an ammonium salt, an alkali metal salt, such as a sodium or a potassium salt, an alkaline earth metal salt, such as a calcium or a magnesium salt, a salt of organic bases such as dicyclohexylamine, N-methyl-D-glucamine, tris(hydroxymethyl)methylamine, C₁-C₇ alkylamine, cyclohexylamine, triethanolamine, ethylenediamine, and salts with amino acids such as arginine and lysine.

As used herein, a “subject” refers to an animal that is the object of treatment, observation or experiment. “Animal” includes cold- and warm-blooded vertebrates and invertebrates such as fish, shellfish, reptiles and, in particular, mammals. “Mammal” includes, without limitation, mice, rats, rabbits, guinea pigs, dogs, cats, sheep, goats, cows, horses, camels, non-human primates, such as monkeys, chimpanzees, and apes, and, in particular, humans. In some embodiments, a subject has an HBV infection. In some embodiments, the subject can be human, for example, a human having an HBV infection.

As used herein, “nadir” refers to where a measured amount of a component of a sample from a subject has decreased to a point where the amount no longer continues to decrease even after additional administrations of an agent. For example, a measured level may be said to be at a nadir when the measured level has not decreased (such as with statistical significance) for a period of at least one week, at least two weeks, at least three weeks, at least four weeks, or more, after at least one additional dose, at least two additional doses, at least three additional doses, at least four additional doses, or more, of an agent. For example, HBsAg levels in a subject may be reduced by administration of a first agent until HBsAg levels reach a nadir, where after administration of at least one additional dose of the first agent and after at least one week, no statistically significant reduction of HBsAg levels is observed.

Terms and phrases used in this application, and variations thereof, especially in the appended claims, unless otherwise expressly stated, should be construed as open ended as opposed to limiting. As examples of the foregoing, the term ‘including’ should be read to mean ‘including, without limitation,’ ‘including but not limited to,’ or the like; the term ‘comprising’ as used herein is synonymous with ‘including,’ ‘containing,’ or ‘characterized by,’ and is inclusive or open-ended and does not exclude additional, unrecited elements or method steps; the term ‘having’ should be interpreted as ‘having at least;’ the term ‘includes’ should be interpreted as ‘includes but is not limited to;’ the term ‘example’ is used to provide exemplary instances of the item in discussion, not an exhaustive or limiting list thereof. In addition, the term “comprising” is to be interpreted synonymously with the phrases “having at least” or “including at least.” When used in the context of a compound or composition, the term “comprising” means that the compound or composition includes at least the recited features or components, but may also include additional features or components.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity. The indefinite article “a” or “an” does not exclude a plurality.

It is understood that, in any compound described herein having one or more chiral centers, if an absolute stereochemistry is not expressly indicated, then each center may independently be of (R)-configuration or (S)-configuration or a mixture thereof. Thus, the compounds provided herein may be enantiomerically pure, enantiomerically enriched, racemic mixture, diastereomerically pure, diastereomerically enriched, or a stereoisomeric mixture. In addition, it is understood that, in any compound described herein having one or more double bond(s) generating geometrical isomers that can be defined as E or Z, each double bond may independently be E or Z a mixture thereof. Likewise, it is understood that, in any compound described, all tautomeric forms are also intended to be included.

It is to be understood that where compounds disclosed herein have unfilled valencies, then the valencies are to be filled with hydrogens or isotopes thereof, for example, hydrogen-1 (protium) and hydrogen-2 (deuterium).

It is understood that the compounds described herein can be labeled isotopically. Substitution with isotopes such as deuterium may afford certain therapeutic advantages resulting from greater metabolic stability, such as, for example, increased in vivo half-life or reduced dosage requirements. Each chemical element as represented in a compound structure may include any isotope of said element. For example, in a compound structure a hydrogen atom may be explicitly disclosed or understood to be present in the compound. At any position of the compound that a hydrogen atom may be present, the hydrogen atom can be any isotope of hydrogen, including but not limited to hydrogen-1 (protium) and hydrogen-2 (deuterium). Thus, reference herein to a compound encompasses all potential isotopic forms unless the context clearly dictates otherwise.

Where a range of values is provided, it is understood that the upper and lower limit, and each intervening value between the upper and lower limit of the range is encompassed within the embodiments.

In an attempt to help the reader of the application, the description has been separated in various paragraphs or sections, or is directed to various embodiments of the application. These separations should not be considered as disconnecting the substance of a paragraph or section or embodiments from the substance of another paragraph or section or embodiments. To the contrary, one skilled in the art will understand that the description has broad application and encompasses all the combinations of the various sections, paragraphs and sentences that can be contemplated. The discussion of any embodiment is meant only to be exemplary and is not intended to suggest that the scope of the invention, including the claims, is limited to these examples. The application contemplates use of any of the applicable components in any combination that can be used the application, whether or not a particular combination is expressly described.

### Methods of Treatment

Some embodiments described herein relate to a method of treating a HBV and/or HDV infection that can include administering to a subject identified as suffering from the HBV and/or HDV infection an effective amount of a compound (such as a first and/or a second agent as described herein), or a pharmaceutically acceptable salt thereof, as described herein, or a pharmaceutical composition that includes an effective amount of a compound, or a pharmaceutically acceptable salt thereof, as described herein. Other embodiments described herein relate to using a compound (such as a first or a second agent as described herein), or a pharmaceutically acceptable salt thereof, as described herein in the manufacture of a medicament for treating a HBV and/or HDV infection. Still other embodiments described herein relate to the use of a compound (such as a first and/or a second agent as described herein), or a pharmaceutically acceptable salt thereof, as described herein or a pharmaceutical composition that includes a compound, or a pharmaceutically acceptable salt thereof, as described herein for treating a HBV and/or HDV infection.

Some embodiments disclosed herein relate to a method of treating a HBV and/or HDV infection that can include contacting a cell infected with the HBV and/or HDV with an effective amount of a compound (such as a first and/or a second agent as described herein), or a pharmaceutically acceptable salt thereof, as described herein, or a pharmaceutical composition that includes an effective amount of a compound, or a pharmaceutically acceptable salt thereof, as described herein. Other embodiments described herein relate to using a compound (such as a first and/or a second agent as described herein), or a pharmaceutically acceptable salt thereof, as described herein in the manufacture of a medicament for treating a HBV and/or HDV infection. Still other embodiments described herein relate to the use of a compound (such as a first and/or a second agent as described herein), or a pharmaceutically acceptable salt thereof, as described herein described herein, or a pharmaceutical composition that includes an effective amount of a compound, or a pharmaceutically acceptable salt thereof, as described herein for treating a HBV and/or HDV infection.

Some embodiments disclosed herein relate to a method of inhibiting replication of HBV and/or HDV that can include contacting a cell infected with the HBV and/or HDV with an effective amount of a compound (such as a first and/or a second agent as described herein), or a pharmaceutically acceptable salt thereof, as described herein, or a pharmaceutical composition that includes an effective amount of a compound, or a pharmaceutically acceptable salt thereof, as described herein. Other embodiments described herein relate to using a compound (such as a first and/or a second agent as described herein), or a pharmaceutically acceptable salt thereof, as described herein in the manufacture of a medicament for inhibiting replication of HBV and/or HDV. Still other embodiments described herein relate to the use of a compound (such as a first and/or a second agent as described herein), or a pharmaceutically acceptable salt thereof, as described herein, or a pharmaceutical composition that includes an effective amount of a compound, or a pharmaceutically acceptable salt thereof, as described herein, for inhibiting replication of HBV and/or HDV.

Provided herein are methods of treating hepatitis B viral and/or hepatitis D viral infection in a subject in need thereof. In some embodiments, the methods include administering an effective amount of a first agent selected from the group consisting of (i) an HBV surface antigen (HBsAg) reducing agent, or a pharmaceutically acceptable salt thereof, and (ii) a capsid assembly modulator (CAM), or a pharmaceutically acceptable salt thereof, to the subject, followed by administering an effective amount of a second agent selected from the group consisting of (i) a CAM, or a pharmaceutically acceptable salt thereof, and (ii) an HBsAg reducing agent, or a pharmaceutically acceptable salt thereof, to the subject. In some embodiments, when the first agent is an HBsAg reducing agent or a pharmaceutically acceptable salt thereof, the second agent is a CAM or a pharmaceutically acceptable salt thereof; and when the first agent is a CAM or a pharmaceutically acceptable salt thereof, the second agent is an HBsAg reducing agent or a pharmaceutically acceptable salt thereof.

In some embodiments, the method is a method of treating an HBV infection. In some embodiments, the method is a method of treating an HDV infection.

In some embodiments, the initial administration of the second agent is after a delay period following initial administration of the first agent. For example, in some embodiments, the initial administration of the second agent is after a delay period following the start of administration of the first agent. For example, in some embodiments, the administration of the second agent is an add-on to a continuous treatment with the first agent.

In some embodiments, provided herein are methods of treating hepatitis B viral and/or hepatitis D viral infection in a subject in need thereof, including administering an effective amount of a first agent selected from the group consisting of (i) an HBV surface antigen (HBsAg) reducing agent, or a pharmaceutically acceptable salt thereof, and (ii) a capsid assembly modulator (CAM), or a pharmaceutically acceptable salt thereof, to the subject, followed by administering an effective amount of a second agent selected from the group consisting of (i) a CAM, or a pharmaceutically acceptable salt thereof, and (ii) an HBsAg reducing agent, or a pharmaceutically acceptable salt thereof, but is not a CAM or a pharmaceutically acceptable salt thereof if the first agent is a CAM or a pharmaceutically acceptable salt thereof, and is not an HBsAg reducing agent or a pharmaceutically acceptable salt thereof if the first agent is a HBsAg reducing agent or a pharmaceutically acceptable salt thereof, to the subject. Thus, in some embodiments, when the first agent is an HBsAg reducing agent or a pharmaceutically acceptable salt thereof, the second agent is a CAM or a pharmaceutically acceptable salt thereof; and when the first agent is a CAM or a pharmaceutically acceptable salt thereof, the second agent is an HBsAg reducing agent or a pharmaceutically acceptable salt thereof. In some embodiments, the initial administration of the second agent is after a delay period following initial administration of the first agent.

In some embodiments, the method is a method of treating an HBV infection. In some embodiments, the method is a method of treating an HDV infection.

Provided herein are methods of maintaining low plasma HBsAg levels in a subject having an HBV and/or an HDV infection. In some embodiments, the methods include administering an effective amount of a first agent selected from the group consisting of (i) an HBV surface antigen (HBsAg) reducing agent, or a pharmaceutically acceptable salt thereof, and (ii) a capsid assembly modulator (CAM), or a pharmaceutically acceptable salt thereof, to the subject, followed by administering an effective amount of a second agent selected from the group consisting of (i) a CAM, or a pharmaceutically acceptable salt thereof, and (ii) an HBsAg reducing agent, or a pharmaceutically acceptable salt thereof, to the subject, wherein when the first agent is an HBsAg reducing agent or a pharmaceutically acceptable salt thereof, the second agent is a CAM or a pharmaceutically acceptable salt thereof; and wherein when the first agent is a CAM or a pharmaceutically acceptable salt thereof, the second agent is an HBsAg reducing agent or a pharmaceutically acceptable salt thereof. In some embodiments the initial administration of the second agent is after a delay period following initial administration of the first agent. In some embodiments, the method is a method of maintaining low plasma HBsAg levels in a subject having an HBV infection. In some embodiments, the method is a method of maintaining low plasma HBsAg levels in a subject having an HDV infection.

In some embodiments, the first agent is an HBsAg reducing agent or a pharmaceutically acceptable salt thereof; and the second agent is a CAM or a pharmaceutically acceptable salt thereof. In some embodiments, the first agent is a short interfering RNA (siRNA), and the second agent is a Class A capsid assembly modulator (CAM A) or a Class E capsid assembly modulator (CAM-E) or a pharmaceutically acceptable salt thereof.

In some embodiments, the first agent is a CAM or a pharmaceutically acceptable salt thereof; and the second agent is an HBsAg reducing agent or a pharmaceutically acceptable salt thereof. In some embodiments, the first agent is a Class A capsid assembly modulator (CAM A) or a Class E capsid assembly modulator (CAM-E) or a pharmaceutically acceptable salt thereof, and the second agent is a short interfering RNA (siRNA).

Further provided herein are improved methods of treating a hepatitis B viral and/or a hepatitis D viral infection in a subject for whom therapy with a first agent selected from the group consisting of (i) an HBV surface antigen (HBsAg) reducing agent, or a pharmaceutically acceptable salt thereof, and (ii) a capsid assembly modulator (CAM), or a pharmaceutically acceptable salt thereof, has been initiated. In some embodiments, the improved method includes administering an effective amount of a second agent that is selected from the group consisting of (i) a CAM, or a pharmaceutically acceptable salt thereof, and (ii) an HBsAg reducing agent, or a pharmaceutically acceptable salt thereof, to the subject, wherein when the first agent is an HBsAg reducing agent or a pharmaceutically acceptable salt thereof, the second agent is a CAM or a pharmaceutically acceptable salt thereof; and wherein when the first agent is a CAM or a pharmaceutically acceptable salt thereof, the second agent is an HBsAg reducing agent or a pharmaceutically acceptable salt thereof. In some embodiments, initial administration of the second agent is after a delay period following initial administration of the first agent. In some embodiments, the improved method is an improved method of treating an HBV infection. In some embodiments, the improved method is an improved method of treating an HDV infection.

In some embodiments, the first agent is an HBsAg reducing agent or a pharmaceutically acceptable salt thereof; and wherein the second agent is a CAM or a pharmaceutically acceptable salt thereof. In some embodiments, the first agent is a CAM or a pharmaceutically acceptable salt thereof; and wherein the second agent is an HBsAg reducing agent or a pharmaceutically acceptable salt thereof.

In some embodiments, the hepatitis B viral (HBV) infection is a chronic hepatitis B viral (HBV) infection.

### Capsid Assembly Modulators (CAMs)

In some embodiments, the CAM is a class A CAM (CAM-A). In some embodiments, the CAM is a class E CAM (CAM-E).

In some embodiments, the CAM is a fused pyrazole compound. Examples of suitable fused pyrazole compounds are described in U.S. Appl. Pub. No. 2022/0169650 A1. In some embodiments, the fused pyrazole compound is a compound of formula (I), or a pharmaceutically acceptable salt thereof:

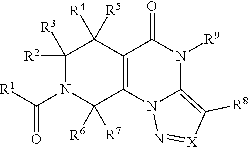

wherein:
X is CH, CD, CF, C(CH₃) or N;
R¹ is a 3,4-substituted phenyl substituted with two moieties independently selected from the group consisting of —Cl, —Br, —CHF₂, —CF₃, —CH₃ and —CN;
R² and R³ are independently selected from the group consisting of hydrogen, an unsubstituted C₁₋₄ alkyl, an unsubstituted C₁₋₄ haloalkyl, an optionally substituted C₃₋₄ cycloalkyl, an optionally substituted aryl, an optionally substituted heteroaryl, an optionally substituted heterocyclyl, an optionally substituted aryl (C₁₋₄ alkyl), an optionally substituted heteroaryl (C₁₋₄ alkyl) and an optionally substituted heterocyclyl (C₁₋₄ alkyl);
R⁴ and R⁵ are independently selected from the group consisting of hydrogen, an unsubstituted C₁₋₄ alkyl, an unsubstituted C₁₋₄ haloalkyl, an optionally substituted aryl, an optionally substituted heteroaryl, an optionally substituted heterocyclyl, an optionally substituted aryl (C₁₋₄ alkyl), an optionally substituted heteroaryl (C₁₋₄ alkyl) and an optionally substituted heterocyclyl (C₁₋₄ alkyl);
R⁶ and R⁷ are independently selected from the group consisting of hydrogen, an unsubstituted C₁₋₄ alkyl and an unsubstituted C₁₋₄ haloalkyl;
R⁸ is —CHR^{8a}R^{8b};
R^{8a} is hydrogen or —CH₃;
R^{8b} is selected from the group consisting of an unsubstituted C₁₋₄ alkyl, an unsubstituted C₂₋₄ alkenyl, an unsubstituted C₂₋₄ alkynyl, an optionally substituted monocyclic C₃₋₆ cycloalkyl, an optionally substituted phenyl, an optionally substituted monocyclic heteroaryl and an optionally substituted monocyclic heterocyclyl; and
R⁹ is a substituted aryl, an optionally substituted heteroaryl or an optionally substituted heterocyclyl.

In some embodiments, the CAM is a fused pyrimidone compound. In some embodiments, the fused pyrimidone compound is of a formula (II), or a pharmaceutically acceptable salt thereof:

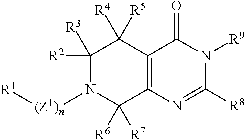

wherein:
n is 0 or 1;
Z¹ is —C(═O)— or —NH—C(═O)—;
R¹ is selected from the group consisting of an optionally substituted aryl, an optionally substituted heteroaryl, an optionally substituted heterocyclyl, an optionally substituted aryl (C₁₋₄ alkyl), an optionally substituted heteroaryl (C₁₋₄ alkyl) and an optionally substituted heterocyclyl (C₁₋₄ alkyl);
R² and R³ are independently selected from the group consisting of hydrogen, an optionally substituted C₁₋₄ alkyl, an optionally substituted C₂₋₄ alkenyl, an optionally substituted C₂₋₄ alkynyl, an unsubstituted C₁₋₄ haloalkyl, an optionally substituted monocyclic C₃₋₆ cycloalkyl, an optionally substituted aryl, an optionally substituted heteroaryl, an optionally substituted heterocyclyl, an optionally substituted aryl (C₁₋₄ alkyl), an optionally substituted heteroaryl (C₁₋₄ alkyl) and an optionally substituted heterocyclyl (C₁₋₄ alkyl);
R⁴ and R⁵ are independently selected from the group consisting of hydrogen, an unsubstituted C₁₋₄ alkyl, an unsubstituted C₁₋₄ haloalkyl, an optionally substituted aryl, an optionally substituted heteroaryl, an optionally substituted heterocyclyl, an optionally substituted aryl (C₁₋₄ alkyl), an optionally substituted heteroaryl (C₁₋₄ alkyl) and an optionally substituted heterocyclyl (C₁₋₄ alkyl);
R⁶ and R⁷ are independently selected from the group consisting of hydrogen, an unsubstituted C₁₋₄ alkyl and an unsubstituted C₁₋₄ haloalkyl;
R⁸ is —NR^{10A}R^{10B}, wherein R^{10A} and R^{10B} are either not directly covalently linked or directly covalently linked to form a 5-6 membered ring, an optionally substituted heteroaryl, or an optionally substituted C₂₋₁₂ alkynyl, wherein the C₂₋₁₂ alkynyl is optionally substituted with one or more substituents selected from the group consisting of amino, —NH—C(═O)(an unsubstituted C₁₋₄ alkyl), hydroxy, an unsubstituted C₁₋₄ alkoxy, an unsubstituted C₁₋₄ haloalkyl, an unsubstituted C₃₋₄ monocyclic cycloalkyl, a fluoro-substituted C₃₋₄ monocyclic cycloalkyl, a hydroxy-substituted C₃₋₄ monocyclic cycloalkyl an unsubstituted 4-6 membered monocyclic heterocyclyl, an optionally substituted aryl and an optionally substituted 5-6 membered monocyclic heteroaryl;
R⁹ is a substituted phenyl, a substituted monocyclic heteroaryl or a substituted fused-bicyclic heteroaryl, wherein the substituted phenyl, the substituted monocyclic heteroaryl or the substituted fused-bicyclic heteroaryl is substituted with one or more substituents selected from the group consisting of halogen, an unsubstituted C₁₋₄ alkyl, a cyano-substituted C₁₋₄ alkyl, an unsubstituted C₁₋₄ haloalkyl, an unsubstituted C₁₋₄ alkoxy, a hydroxy-substituted C₁₋₄ alkoxy, an optionally substituted monocyclic C₃₋₆ cycloalkyl, an optionally substituted monocyclic heteroaryl, an optionally substituted monocyclic heterocyclyl, amino, a mono-substituted amine, a di-substituted amine and —C(═O)NHR¹¹;
R^{10A} is hydrogen, an unsubstituted C₁₋₆ alkyl, a monocyclic C₃₋₆ cycloalkyl optionally substituted with one or two halogens, an optionally substituted 5-6 membered monocyclic heteroaryl, an optionally substituted 4-6 membered monocyclic heterocyclyl or an optionally substituted monocyclic C₃₋₆ cycloalkyl (C₁₋₄ alkyl);

R^{10B} is selected from the group consisting of an optionally substituted C₂₋₈ alkenyl, an optionally substituted C₂₋₈ alkynyl, an optionally substituted aryl, an optionally substituted aryl (C₁₋₄ alkyl), an optionally substituted heteroaryl (C₁₋₄ alkyl) and an optionally substituted heterocyclyl (C₁₋₄ alkyl), wherein the C₂₋₈ alkenyl and the C₂₋₈ alkynyl is optionally substituted with one or more substituents selected from the group consisting of amino, hydroxy, an unsubstituted C₁₋₄ alkoxy, an unsubstituted C₁₋₄ haloalkyl, an unsubstituted C₃₋₄ monocyclic cycloalkyl, a fluoro-substituted C₃₋₄ monocyclic cycloalkyl, a hydroxy-substituted C₃₋₄ monocyclic cycloalkyl and an unsubstituted 4-6 membered monocyclic heterocyclyl;
wherein when R^{10A} is an optionally substituted monocyclic C₃₋₆ cycloalkyl (C₁₋₄ alkyl), then R^{10B} cannot be an unsubstituted C₂₋₆ alkenyl; and
R¹¹ is hydrogen, an unsubstituted C₁₋₆ alkyl, an optionally substituted C₂₋₆ alkenyl, an optionally substituted C₁₋₆ alkynyl or an optionally substituted C₃₋₆ monocyclic cycloalkyl.

In some embodiments, the CAM is a pyrrole compound. Examples of suitable pyrrole compounds are described in U.S. Pat. No. 11,191,747. In some embodiments, the pyrrole compound is a compound of formula (I), or a pharmaceutically acceptable salt thereof:

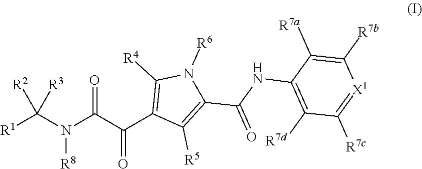

wherein:
R¹ is an unsubstituted or a substituted C₂ alkenyl, an unsubstituted or a substituted C₂ alkynyl, an unsubstituted or a substituted monocyclic heteroaryl, an unsubstituted or a substituted bicyclic heteroaryl or an unsubstituted or a substituted monocyclic heterocyclyl, wherein when the C₂ alkenyl, the C₂ alkynyl, an unsubstituted C₁₋₄ haloalkyl and the monocyclic heteroaryl are substituted, the C₂ alkenyl, the C₂ alkynyl and the monocyclic heteroaryl are independently substituted with one or more substituents selected from the group consisting of halogen, an unsubstituted C₁₋₄ alkyl, an unsubstituted C₁₋₄ haloalkyl, an unsubstituted C₁₋₄ hydroxyalkyl, an unsubstituted monocyclic C₃₋₆ cycloalkyl and a hydroxy-substituted monocyclic C₃₋₆ cycloalkyl;
R² and R³ are independently selected from the group consisting of hydrogen, an unsubstituted or a substituted C₁₋₄ alkyl, an unsubstituted C₁₋₄ haloalkyl, an unsubstituted or a substituted monocyclic C₃₋₆ cycloalkyl, an unsubstituted or a substituted monocyclic 3-6 membered heterocyclyl, an unsubstituted C₁₋₄ hydroxyalkyl and an unsubstituted C₁₋₅ alkoxyalkyl, wherein when the monocyclic C₃₋₆ cycloalkyl and the monocyclic 3-6 heterocyclyl are substituted, the monocyclic C₃₋₆ cycloalkyl and the monocyclic 3-6 heterocyclyl are independently substituted with one or more substituents selected from the group consisting of halogen or hydroxy, and wherein when the C₁₋₄ alkyl is substituted, the C₁₋₄ alkyl is substituted with one or more substituents selected from the group consisting of a phosphate, an O-linked α-amino acid and an O-carboxy, and provided that at least one of R² and R³ is not hydrogen; or
R² and R³ are taken together along with the carbon to which R² and R³ are attached to form an unsubstituted or a substituted monocyclic C₃₋₆ cycloalkyl or an unsubstituted or a substituted monocyclic 3-6 membered heterocyclyl, wherein when the C₃₋₆ cycloalkyl and 3-6 membered heterocyclyl are substituted, the C₃₋₆ cycloalkyl and the 3-6 membered heterocyclyl are independently substituted with 1 or 2 substituents selected from the group consisting of halogen and hydroxy;
R⁴ and R⁵ are independently hydrogen, halogen, an unsubstituted C₁₋₄ alkyl, a deuterated C₁₋₄ alkyl or an unsubstituted C₂₋₄ alkenyl;
R⁶ is hydrogen, an unsubstituted C₁₋₄ alkyl, a deuterated C₁₋₄ alkyl or an unsubstituted C₃₋₄ alkenyl; and
provided that at least one of R⁴, R⁵ and R⁶ is not hydrogen; or
R⁵ is hydrogen, halogen, an unsubstituted C₁₋₄ alkyl or an unsubstituted C₂₋₄ alkenyl; and R⁴ and R⁶ are taken together to form an unsubstituted or substituted 5-6 membered heterocyclic ring;
X¹ is CRA or N;
R^{7a}, R^{7b}, R^{7c} and R^{7d} are independently hydrogen, halogen, an unsubstituted C₁₋₄ haloalkyl, cyano or an unsubstituted C₁₋₄ alkoxy;
R⁸ is hydrogen, —CH₂OC(═O)-(an unsubstituted C₁₋₄ alkyl), —CH₂OC(═O)—O(an unsubstituted C₁₋₄ alkyl), —CH₂—(α-amino acid) or —CH₂-phosphate; and
R^{A} is hydrogen, halogen, an unsubstituted C₁₋₄ haloalkyl or cyano.

In some embodiments, the CAM is selected from the compounds of Table A. Preferably the CAM is a substantially pure version of a single compound; the CAM may be a combination containing one of more compounds disclosed herein as a CAM.

TABLE A

| Compound No. | Compound |
|---|---|
| 1 | 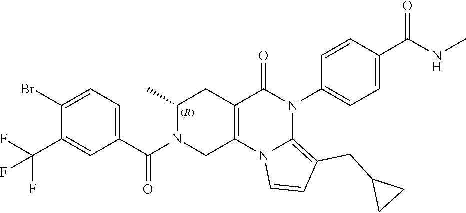 |
| | N-methyl-4-[(11R)-12-[4-bromo-3-(trifluoromethyl)benzoyl]-5- |
| | (cyclopropylmethyl)-11-methyl-8-oxo-2,3,7,12- |
| | tetrazatricyclo[7.4.0.0∧2,6]trideca-1(9),3,5-trien-7-yl]benzamide |
| 2 | 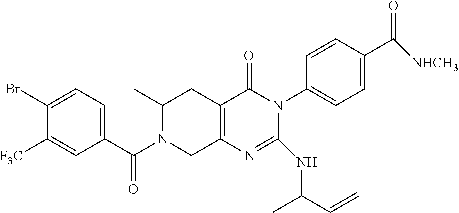 [SMILES: C=CC(C)Nc1nc2c(c(=O)n1-c1ccc(C(=O)NC)cc1)CC(C)N(C(=O)c1ccc(Br)c(C(F)(F)F)c1)C2] |
| | (version showing atomic connectivity but not stereochemistry) |
| | 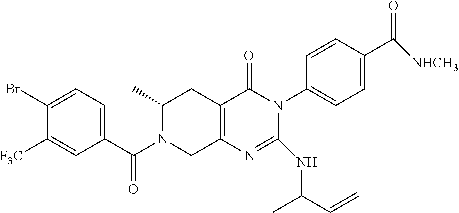 [SMILES: C=CC(C)Nc1nc2c(c(=O)n1-c1ccc(C(=O)NC)cc1)C[C@@H](C)N(C(=O)c1ccc(Br)c(C(F)(F)F)c1)C2] |
| | (version showing stereochemistry) |
| | 4-((6R)-7-(4-bromo-3-(trifluoromethyl)benzoyl)-2-(but-3-en-2- |
| | ylamino)-6-methyl-4-oxo-5,6,7,8-tetrahydropyrido[3,4- |
| | d]pyrimidin-3(4H)-yl)-N-methylbenzamide |
| 3 | 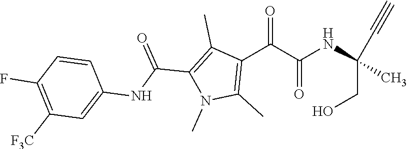 |
| | N-[4-fluoro-3-(trifluoromethyl)phenyl]-1,3,5-trimethyl-4-[2-oxo- |
| | 2-[[(1S)-1-(hydroxymethyl)-1-methyl-prop-2- |
| | ynyl]amino]acetyl]pyrrole-2-carboxamide |
| 4 | 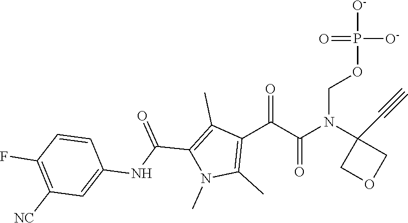 [SMILES: C#CC1(N(COP(=O)([O-])[O-])C(=O)C(=O)c2c(C)c(C(=O)Nc3ccc(F)c(C#N)c3)n(C)c2C)COC1] |
| | [[2-[5-[(3-cyano-4-fluoro-phenyl)carbamoyl]-1,2,4-trimethyl- |
| | pyrrol-3-yl]-2-oxo-acetyl]-(3-ethynyloxetan-3-yl)amino]methyl |
| | dihydrogen phosphate |
| 5 | 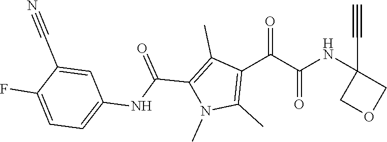 [SMILES: C#CC1(NC(=O)C(=O)c2c(C)c(C(=O)Nc3ccc(F)c(C#N)c3)n(C)c2C)COC1] |
| | N-(3-cyano-4-fluoro-phenyl)-4-[2-[(3-ethynyloxetan-3-yl)amino]- |
| | 2-oxo-acetyl]-1,3,5-trimethyl-pyrrole-2-carboxamide |
| 6 | 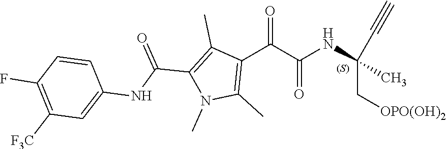 |
| | (S)-2-(2-(5-((4-fluoro-3-(trifluoromethyl)phenyl)carbamoyl)- |
| | 1,2,4-trimethyl-1H-pyrrol-3-y1)-2-oxoacetamido)-2-methylbut-3- |
| | yn-1-yl dihydrogen phosphate |
| 7 | 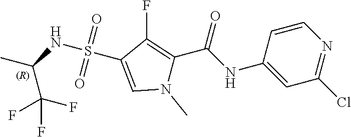 |
| | (R)-N-(2-chloropyridin-4-yl)-3-fluoro-1-methyl-4-(N-(1,1,1- |
| | trifluoropropan-2-y1)sulfamoyl)-1H-pyrrole-2-carboxamide |
| 8 | 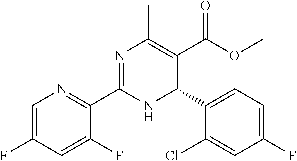 [SMILES: COC(=O)C1=C(C)N=C(c2ncc(F)cc2F)N[C@H]1c1ccc(F)cc1Cl] |
| | BAY 41-4109 |
| 9 | 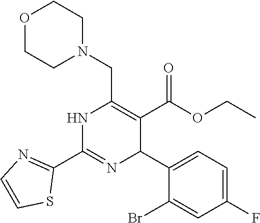 [SMILES: CCOC(=O)C1=C(CN2CCOCC2)NC(c2nccs2)=NC1c1ccc(F)cc1Br] |
| | GLS4 (mophothiadin) |
| 10 | 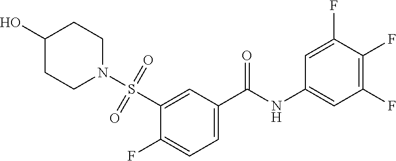 [SMILES: O=C(Nc1cc(F)c(F)c(F)c1)c1ccc(F)c(S(=O)(=O)N2CCC(O)CC2)c1] |
| | NVR 3-778 |
| 11 | 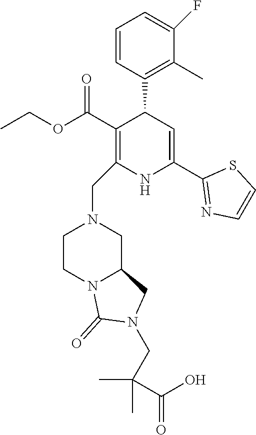 [SMILES: CCOC(=O)C1=C(CN2CCN3C(=O)N(CC(C)(C)C(=O)O)C[C@@H]3C2)NC(c2nccs2)=C[C@H]1c1cccc(F)c1C] |
| | RG7907 (RO7049389) |
| 12 | 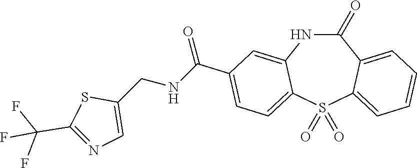 [SMILES: O=C(NCc1cnc(C(F)(F)F)s1)c1ccc2c(c1)NC(=O)c1ccccc1S2(=O)=O] |
| | ABI-H0731 (vebicorvir) |
| 13 | ABI-3773 (Assembly Biosciences) |
| 14 | ABI-4334 (Assembly Biosciences) |
| 15 | 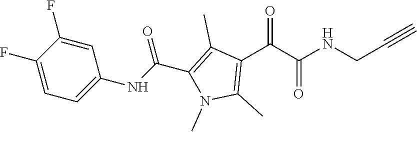 |
| | GLP-26 |
| 16 | KL-060332 (Sichuan Kelun Pharmaceutical) |
| 17 | 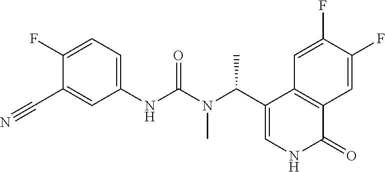 [SMILES: C[C@H](c1c[nH]c(=O)c2cc(F)c(F)cc12)N(C)C(=O)Nc1ccc(F)c(C#N)c1] |
| | AB-836 (Arbutus) |
| 18 | VNRX-9945 (Venatorx) |
| 19 | 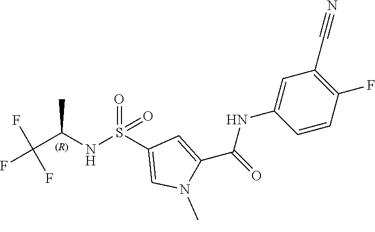 |
| | JNJ-56136379 (JNJ-6379) |
| | (R)-N-(3-cyano-4-fluorophenyl)-1-methy1-4-(N-(1,1,1- |
| | trifluoropropan-2-yl)sulfamoyl)-1H-pyrrole-2-carboxamide |
| 20 | JNJ-64530440 (JNJ-0440) |
| 21 | EDP-514 (Enanta Pharmaceuticals) |
| 22 | 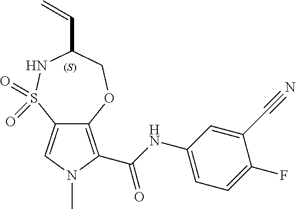 |
| | - IZ |
| | (S)-N-(3-cyano-4-fluorophenyl)-7-methyl-3-vinyl-3,4-dihydro- |
| | 2H,7H-pyrrolo[3,4-b][1,4,5]oxathiazepine-6-carboxamide 1,1- |
| | dioxide |
| 23 | 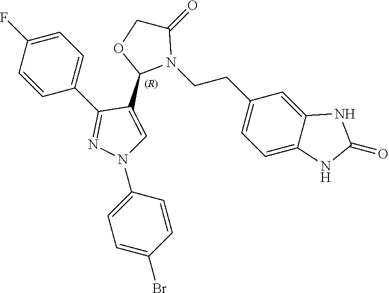 |
| | ZM-H150SR |
| 24 | 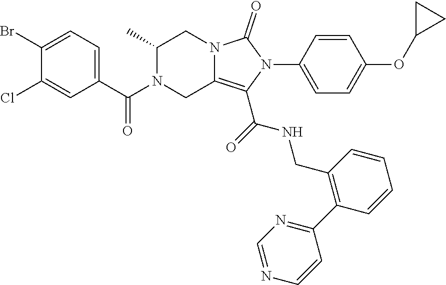 [SMILES: C[C@@H]1Cn2c(c(C(=O)NCc3ccccc3-c3ccncn3)n(-c3ccc(OC4CC4)cc3)c2=O)CN1C(=O)c1ccc(Br)c(Cl)c1] |
| | (R)-7-(4-bromo-3-chlorobenzoyl)-2-(4-cyclopropoxyphenyl)-6- |
| | methyl-3-oxo-N-(2-(pyrimidin-4-yl)benzyl)-2,3,5,6,7,8- |
| | hexahydroimidazo[ 1,5-a]pyrazine-1-carboxamide |
| 25 | 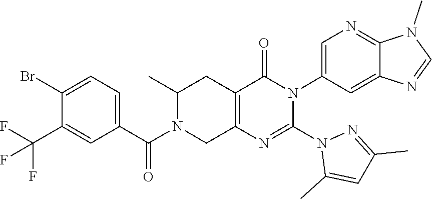 [SMILES: Cc1cc(C)n(-c2nc3c(c(=O)n2-c2cnc4c(c2)ncn4C)CC(C)N(C(=O)c2ccc(Br)c(C(F)(F)F)c2)C3)n1] |
| | (version showing atomic connectivity but not stereochemistry) |
| | 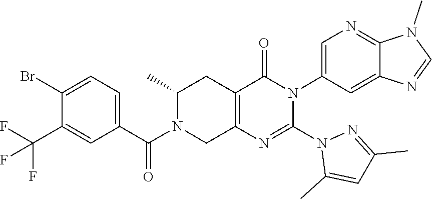 [SMILES: Cc1cc(C)n(-c2nc3c(c(=O)n2-c2cnc4c(c2)ncn4C)C[C@@H](C)N(C(=O)c2ccc(Br)c(C(F)(F)F)c2)C3)n1] |
| | (version showing stereochemistry) |
| | (6R)-7-[4-bromo-3-(trifluoromethyl)benzoyl]-2-(3,5- |
| | dimethylpyrazol-1-yl)-6-methyl-3-(3-methylimidazo[4,5- |
| | b]pyridin-6-yl)-6,8-dihydro-5H-pyrido[3,4-d]pyrimidin-4-one |
| 26 | 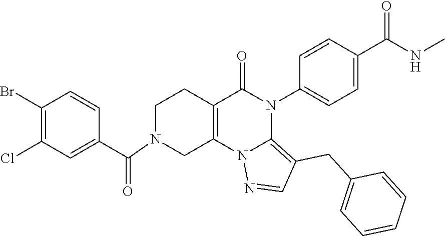 [SMILES: CNC(=O)c1ccc(-n2c(=O)c3c(n4ncc(Cc5ccccc5)c24)CN(C(=O)c2ccc(Br)c(Cl)c2)CC3)cc1] |
| | 4-[5-benzyl-12-(4-bromo-3-chloro-benzoyl)-8-oxo-2,3,7,12- |
| | tetrazatricyclo[7.4.0.0?2,6]trideca-1(9),3,5-trien-7-yl]-N-methyl- |
| | benzamide |
| 27 | 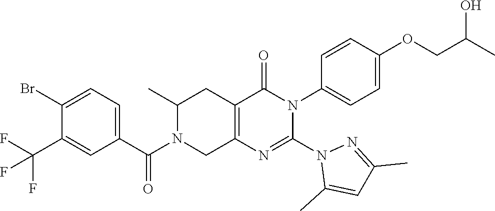 [SMILES: Cc1cc(C)n(-c2nc3c(c(=O)n2-c2ccc(OCC(C)O)cc2)CC(C)N(C(=O)c2ccc(Br)c(C(F)(F)F)c2)C3)n1] |
| | (version showing atomic connectivity but not stereochemistry) |
| | 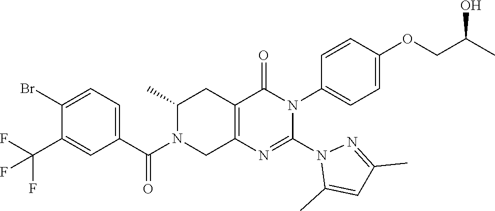 [SMILES: Cc1cc(C)n(-c2nc3c(c(=O)n2-c2ccc(OC[C@H](C)O)cc2)C[C@@H](C)N(C(=O)c2ccc(Br)c(C(F)(F)F)c2)C3)n1] |
| | (version showing stereochemistry) |
| | (6R)-7-[4-bromo-3-(trifluoromethyl)benzoyl]-2-(3,5- |
| | dimethylpyrazol-1-y1)-3-[4-[(2S)-2-hydroxypropoxy]phenyl]-6- |
| | methyl-6,8-dihydro-5H-pyrido[3,4-d]pyrimidin-4-one |

In some embodiments, the CAM is selected from the group consisting of N-methyl-4+[11R)-2-[4-bromo-3-(trifluoromethyl)benzoyl]-5-(cyclopropylmethyl)-11-methyl-8-oxo-2,3,7,12-tetrazatricyclo[7.4.0.0\hat{ }2,6]trideca-1(9),3,5-trien-7-yl]benzamide (Compound 1); 4-((R)-7-(4-bromo-3-(trifluoromethyl)benzoyl)-2-(((S)-but-3-en-2-yl)amino)-6-methyl-4-oxo-5,6,7,8-tetrahydropyrido[3,4-d[pyrimidin-3(4H)-yl)-N-methylbenzamide (Compound 2); N-[4-fluoro-3-(trifluoromethyl)phenyl]-1,3,5-trimethyl-4-[2-oxo-2-[[(1S)-1-(hydroxymethyl)-1-methyl-prop-2-ynyl]amino]acetyl]pyrrole-2-carboxamide (Compound 3); [[2-[5-[(3-cyano-4-fluoro-phenyl)carbamoyl]-1,2,4-trimethyl-pyrrol-3-yl]-2-oxo-acetyl]-(3-ethynyloxetan-3-yl)amino]methyl dihydrogen phosphate (Compound 4); N-(3-cyano-4-fluoro-phenyl)-4-[2-[(3-ethynyloxetan-3-yl)amino]-2-oxo-acetyl]-1,3,5-trimethyl-pyrrole-2-carboxamide (Compound 5); (S)-2-(2-(5-((4-fluoro-3-(trifluoromethyl)phenyl)carbamoyl)-1,2,4-trimethyl-1H-pyrrol-3-yl)-2-oxoacetamido)-2-methylbut-3-yn-1-yl dihydrogen phosphate (Compound 6); (R)-N-(2-chloropyridin-4-yl)-3-fluoro-1-methyl-4-(N-(1,1,1-trifluoropropan-2-yl)sulfamoyl)-1H-pyrrole-2-carboxamide (Compound 7); BAY 41-4109 (Compound 8); GLS4 (Compound 9); NVR 3-778 (Compound 10); RG7907 (Compound 11); ABI-H0731 (Compound 12); ABI-3773 (Compound 13); ABI-4334 (Compound 14); GLP-26 (Compound 15); KL-060332 (Compound 16); AB-836 (Compound 17); VNRX-9945 (Compound 18); (R)-N-(3-cyano-4-fluorophenyl)-1-methyl-4-(N-(1,1,1-trifluoropropan-2-yl)sulfamoyl)-1H-pyrrole-2-carboxamide (Compound 19); JNJ-64530440 (Compound 20); EDP-514 (Compound 21); (S)-N-(3-cyano-4-fluorophenyl)-7-methyl-3-vinyl-3,4-dihydro-2H,7H-pyrrolo[3,4-b][1,4,5]oxathiazepine-6-carboxamide 1,1-dioxide (Compound 22); ZM-H1505R (Compound 23); (R)-7-(4-bromo-3-chlorobenzoyl)-2-(4-cyclopropoxyphenyl)-6-methyl-3-oxo-N-(2-(pyrimidin-4-yl)benzyl)-2,3,5,6,7,8-hexahydroimidazo[1,5-a]pyrazine-1-carboxamide (Compound 24); (6R)-7-[4-bromo-3-(trifluoromethyl)benzoyl]-2-(3,5-dimethylpyrazol-1-yl)-6-methyl-3-(3-methylimidazo[4,5-b]pyridin-6-yl)-6,8-dihydro-5H-pyrido[3,4-d]pyrimidin-4-one (Compound 25); 4-[5-benzyl-12-(4-bromo-3-chloro-benzoyl)-8-oxo-2,3,7,12-tetrazatricyclo[7.4.0.0\hat{ }2,6]trideca-1(9),3,5-trien-7-yl]-N-methyl-benzamide (Compound 26); and (6R)-7-[4-bromo-3-(trifluoromethyl)benzoyl]-2-(3,5-dimethylpyrazol-1-yl)-3-[4-[(2S)-2-hydroxypropoxy]phenyl]-6-methyl-6,8-dihydro-5H-pyrido[3,4-d]pyrimidin-4-one (Compound 27).

In some embodiments, the CAM is selected from any of the CAMs disclosed in WO 2017/156255 A1; WO 2020/205934 A1; WO 2015/138895 A1; WO 2019/241292 A1; WO 2019/154343 A1; Zlotnick et al., 2002, A Small Molecule Inhibits and Misdirects Assembly of Hepatitis B Virus Capsids, J. Virol., 76:4848-4854; WO 2018/172852 A1, WO 2018/090862 A1; Deres et al., 2003, Inhibition of Hepatitis B Virus Replication by Drug-Induced Depletion of Nucleocapsids, Science, 299:893-896; WO 2013/096744 A1; WO 2008/154817 A1; WO 2016/161268 A1; WO 2018/039531 A1; WO 2013/144129 A1; WO 2014/033176; WO2020182990A1; WO2020182990A1; WO2022053010; WO2020125729; WO2022115384; US20220119385; WO2022081758; US20220000874; WO2021030278; WO2020247504; WO2020243199; WO2020214728; WO2020167984; WO2022266193; WO2023205653; or WO2023205645; which are each hereby incorporated by reference in their entirety.

### HBV Surface Antigen (HBsAe) Reducing Agent

In some embodiments, the HBsAg reducing agent is a small interfering RNA (siRNA). Examples of suitable siRNAs are described in US 2022/0177888 A1.

In some embodiments, the siRNA is a compound selected from the group consisting of RG6346 (Roche/Dicerna), ARC-520 (Arrowhead), ARC-521 (Arrowhead), ALN-HBV (Alnylam/VIR), VIR-2218 (Alnylani/VIR), AB-729 (Arbutus), and JNJ3989 (Arrowhead/JNJ).

In some embodiments, the siRNA has a nucleic sequence asset forth in SEQ ID NO: 2, SEQ ID NO: 3, SEQ ID NO: 4, SEQ ID NO: 5, SEQ ID NO: 6, or SEQ ID NO: 7. These sequences are set forth below in Table C, with modifications as described in Table B.

TABLE B

| Abbreviation | Modification |
|---|---|
| m | 2′-O-methyl ribose |
| ln | 2′-4′ locked nucleic acid |
| ps | phosphorothioate linkage |
| cp | cyclopropyl 2′-4′ locked nucleic acid |
| f | 2′-fluoro ribose |

TABLE C

| SEQ ID | Sense/ | |
|---|---|---|
| NO | Antisense | Sequence (5′-3′) |
| 2 | siRNA-1 | mC psmC psmG mU fG mU fG |
| | Sense | fC fA mC mU mU mC mG mC |
| | | mU mU mC mA |
| 3 | siRNA-1 | mU psfG psmA mA fG mC mG |
| | Antisense | fA mA mG mU mG mC fA mC |
| | | mA fC mG mGps mU psmC |
| 4 | siRNA-2 | mG psmU psmG mG fU mG fG |
| | Sense | fA fC mU mU mC mU mC mU |
| | | mC mA mA mU |
| 5 | siRNA-2 | mA psfU psmU mG mA fG mA |
| | Antisense | mG mA mA mG mU mC fC mA |
| | | fC mC mA mC psmA psmA |
| 6 | siRNA-3 | mG psmU psmG mG fU mG fG |
| | Sense | fA fC mU mU mC mU mC mU |
| | | mC mA mA mU |
| 7 | siRNA-3 | mA psfU psmU mG mA fG mA |
| | Antisense | mG mA mA mG mU mC fC mA |
| | | fC mC mA mC psmG psmA |

In some embodiments, the HBsAg reducing agent is an siRNA selected from any of the siRNAs disclosed in WO 2016/077321 A1; WO 2020/163747 A1; US 2022/0177888 A1; US 2004/0127446 A1; WO 2018/191278 A2; U.S. Pat. No. 8,202,979 B2; WO 2013/003520 A1; U.S. Pat. No. 8,349,809 B2; or WO2023039005; which are each hereby incorporated by reference in their entirety.

In some embodiments, the HBsAg reducing agent is an antisense oligonucleotide (ASO). In some embodiments, the ASO is selected from the group consisting of GSK-404 (Isis/GlaxoSmithKline), GSK-836 (Isis/GlaxoSmithKline), and RG6004 (Roche).

In some embodiments, the ASO has a nucleic acid sequence as set forth in SEQ ID NO: 1. This sequence is set forth below in Table E, with modifications as described in Table D.

TABLE D

| Abbreviation | Modification |
|---|---|
| m | 2′-O-methyl deoxyribose |
| ln | 2′-4′ locked nucleic acid |
| ps | phosphorothioate linkage |
| (5oh)C | 5-hydroxy Cytidine base |
| (5m)C | 5-methyl Cytidine base |
| cp | cyclopropyl 2′-4′ locked nucleic acid |
| f | 2′-fluoro deoxyribose |

TABLE E

| SEQ ID | |
|---|---|
| NO | Sequence (5′-3′) |
| 1 | mA lnG pslnA pslnT pslnA pslnA psA |
| | psA ps(5oh)C psG ps(5m)C ps(5m)C psG |
| | ps(5m)C pslnA pslnG pslnA pscp(5m)C |

In some embodiments, the HBsAg reducing agent is an ASO selected from any of the ASOs disclosed in WO 97/003211; WO 2012/145697 A1; WO 2017/021385; WO 2018/053185 A1; U.S. Pat. No. 10,793,859 B2; or U.S. Pat. No. 11,466,274 B2; or WO2023177808; which are each hereby incorporated by reference in their entirety.

In some embodiments, the HBsAg reducing agent is a nucleic acid polymer (NAP). In some embodiments, the NAP is a S-antigen Transport-inhibiting Oligonucleotide Polymer (STOP). In some embodiments, the NAP is REP-2139 (Replicor).

In some embodiments, the HBsAg reducing agent is a NAP is selected from any of the NAPs disclosed in WO 2004/024919 A1; US 2004/0162253 A1; WO 2016/030863 A1; US 2020/0147124 A1; or WO 2021/198958 A1; which are each hereby incorporated by reference in their entirety.

### Delay Period

In some embodiments, the initial administration of the second agent (for example, a CAM, siRNA or ASO) is after a delay period following the initial administration of the first agent (for example, a CAM, siRNA or ASO). In some embodiments, the initial administration of the second agent is after a delay period following the final administration of the first agent, for example, when the first agent is administered multiple times. In some embodiments, the initial administration of the second agent is after a delay period following the start of administration of the first agent. For example, in some embodiments, the administration of the second agent is an add-on to a continuous treatment with the first agent.

In some embodiments, administration is repeated after the initial administration. For example, in some embodiments the first agent or the second agent is continuously administered after the initial administration of the first agent or the second agent. In some embodiments, continuous administration refers to administration of the first agent or the second agent on regular, repeating intervals. For example, in some embodiments, continuous administration means administration once a day every day, twice a day every day, three times a day every day, four times a day every day, or five or more times a day every day, or a range constructed from any of the aforementioned values. In some embodiments, continuous administration means administration once every other day, once every three days, once every four days, once every five days, once every six days, once every week, or once every other week, or a range constructed from any of the aforementioned values. In some embodiments, continuous administration means administration about every 6 hours, about every 7 hours, about every 8 hours, about every 9 hours, about every 10 hours, about every 11 hours, about every 12 hours, about every 13 hours, about every 14 hours, about every 15 hours, about every 16 hours, about every 17 hours, about every 18 hours, about every 19 hours, about every 20 hours, about every 21 hours, about every 22 hours, about every 23 hours, about every 24 hours, and so on, or a range constructed from any of the aforementioned values.

In some embodiments, initial administration of the second agent occurs after the first agent has been administered (in some embodiments, continuously) for a period of time (in other words, a delay period), for example at least two weeks, at least a month, at least six weeks at least two months, at least three months, at least four months, at least five months, or at least six months.

In some embodiments, the initial administration of the second agent is after a delay period following the initial (such as the start of) administration of the first agent. In some embodiments, the delay period following the initial administration of the first agent is greater than approximately 1 day. In some embodiments, the delay period is greater than approximately 2 days, approximately 3 days, approximately 4 days, approximately 5 days, approximately 6 days, approximately 7 days, approximately 8 days, approximately 9 days, approximately 10 days, approximately 11 days, approximately 12 days, approximately 13 days, approximately 14 days, approximately 15 days, approximately 16 days, approximately 17 days, approximately 18 days, approximately 19 days, approximately 20 days, approximately 21 days, approximately 22 days, approximately 23 days, approximately 24 days, approximately 25 days, approximately 26 days, approximately 27 days, approximately 28 days, approximately 29 days, approximately 30 days, approximately 31 days, approximately 32 days, approximately 33 days, approximately 34 days, approximately 35 days, approximately 36 days, approximately 37 days, approximately 38 days, approximately 39 days, approximately 40 days, approximately 41 days, approximately 42 days, approximately 43 days, approximately 44 days, approximately 45 days, approximately 46 days, approximately 47 days, approximately 48 days, approximately 49 days, approximately 50 days, approximately 51 days, approximately 52 days, approximately 53 days, approximately 54 days, approximately 55 days, approximately 56 days, approximately 57 days, approximately 58 days, approximately 59 days, approximately 60 days, approximately 61 days, approximately 62 days, approximately 63 days, approximately 64 days, approximately 65 days, approximately 66 days, approximately 67 days, approximately 68 days, approximately 69 days, approximately 70 days, approximately 71 days, approximately 72 days, approximately 73 days, approximately 74 days, approximately 75 days, approximately 76 days, approximately 77 days, approximately 78 days, approximately 78 days, approximately 79 days, approximately 80 days, approximately 81 days, approximately 82 days, approximately 83 days, approximately 84 days, approximately 85 days, approximately 86 days, approximately 87 days, approximately 88 days, approximately 89 days, approximately 90 days, approximately 91 days, approximately 92 days, approximately 93 days, approximately 94 days, approximately 95 days, approximately 96 days, approximately 97 days, approximately 98 days, approximately 99 days, approximately 100 days, or more, or a range constructed from any of the aforementioned values. In some embodiments, the delay period is at least approximately 70 days. In some embodiments, the delay period is at least approximately 50 days.

In some embodiments, the delay period is approximately 1 week, approximately 2 weeks, approximately 3 weeks, approximately 4 weeks, approximately 5 weeks, approximately 6 weeks, approximately 7 weeks, approximately 8 weeks, approximately 9 weeks, approximately 10 weeks, approximately 11 weeks, approximately 12 weeks, approximately 13 weeks, approximately 14 weeks, approximately 15 weeks, approximately 16 weeks, approximately 17 weeks, approximately 18 weeks, approximately 19 weeks, approximately 20 weeks, approximately 21 weeks, approximately 22 weeks, approximately 23 weeks, approximately 24 weeks, approximately 25 weeks, approximately 26 weeks, approximately 27 weeks, approximately 28 weeks, approximately 29 weeks, approximately 30 weeks, approximately 31 weeks, approximately 32 weeks, approximately 33 weeks, approximately 34 weeks, approximately 35 weeks, approximately 36 weeks, approximately 37 weeks, approximately 38 weeks, approximately 39 weeks, approximately 40 weeks, approximately 41 weeks, approximately 42 weeks, approximately 43 weeks, approximately 44 weeks, approximately 45 weeks, approximately 46 weeks, approximately 47 weeks, approximately 48 weeks, approximately 49 weeks, approximately 50 weeks, approximately 51 weeks, approximately 52 weeks, approximately 53 weeks, approximately 54 weeks, or a range constructed from any of the aforementioned values.

In some embodiments, an effective amount of a HBsAg reducing agent is administered to the subject, followed by administering an effective amount of a capsid assembly modulator CAM to the subject. In some embodiments, the initial administration of the CAM is after a delay period following the initial administration of the HBsAg reducing agent. In some embodiments, the delay period is any of the delay periods provided herein.

In some embodiments, the first agent or the second agent is administered multiple times, such as two times, three times, four times, five times, six times, seven times, etc., with an additional interval of delay between each administration. The interval between administrations of the first agent or the second agent may be any of the above-mentioned times listed for the delay period. For example, in some embodiments, the first agent is administered at least three times at regular intervals before administration of the second agent.

In some embodiments, initial administration of the second agent occurs after HBsAg levels have been reduced in the subject by administration of the first agent. For example, in some embodiments, the delay period is determined based on measurement of the subject's plasma HBsAg levels. In some embodiments, the delay period extends until the subject's HBsAg levels are reduced compared to pre-treatment baseline HBsAg levels. For example, in some embodiments, the delay period extends until a 10-fold reduction in HBsAg levels as compared to baseline has been attained. In other words, in such an embodiment, (in other words, add-on of HBsAg reducing agent such as siRNA or ASO is initiated when a ten-fold reduction in HBsAg levels as compared to baseline has been attained. In some embodiments, the delay period extends until the reduction in the subject's HBsAg levels as compared to baseline is about two-fold, about three-fold, about four-fold, about five-fold, about six-fold, about seven-fold, about eight-fold, about nine-fold, about ten-fold, about eleven-fold, about twelve-fold, about thirteen fold, about fourteen fold, about fifteen-fold, about sixteen-fold, about seventeen-fold, about eighteen-fold, about nineteen-fold, about twenty-fold, about thirty-fold, about forty-fold, about fifty-fold, about sixty-fold, about seventy-fold, about eighty-fold, about ninety-fold, or about a hundred-fold, or a range constructed from any of the aforementioned values. In some embodiments, the delay period extends until there is about a 0.5 log 10 reduction, about a 1 log 10 reduction, about a 1.5 log 10 reduction, about a 2 log 20 reduction, about a 2.5 log 10 reduction, about a 3 log 10 reduction, about a 3.5 log 10 reduction, about a 4 log 10 reduction, or about a 5 log 10 reduction in the subject's HBsAg levels as compared to baseline. Thus, in some embodiments, the methods may include regularly (such as daily) measuring a subject's HBsAg levels, such as to use for comparison to baseline.

In some embodiments, initial administration of the second agent occurs after HBsAg levels have been reduced to a nadir in the subject by administration of the first agent. In some embodiments, the nadir comprises a period of at least one week including at least one additional dose of the first agent, wherein the at least one additional dose results in no statistically significant reduction of HBsAg levels. In some embodiments, the delay period extends until the subject's plasma HBsAg levels reach a nadir, for example until the subject's plasma HBsAg levels do not continue to decrease, even after at least one, at least two, at least three, at least four, at least five, or more administrations of the first agent, and after at least one day, at least two days, at least three days, at least one week, at least two weeks, at least three weeks, at least four weeks, at least five weeks, or more. In some embodiments, the delay period extends until the subject's plasma HBsAg levels do not further decrease after at least two additional administrations of the first agent, and after at least two weeks, or at least three weeks.

In some embodiments, the delay period is determined based on measurement of another HBV marker such as HBV RNA, HBV DNA, HBeAg, HBcrAg. For example, in some embodiments, the delay period extends until the subject's other HBV marker levels are reduced compared to pre-treatment baseline other HBV marker levels. For example, in some embodiments, the delay period extends until a 10-fold reduction in other HBV marker levels as compared to baseline has been attained. In other words, in such an embodiment, (in other words, add-on of HBsAg reducing agent such as siRNA or ASO is initiated when a ten-fold reduction in other HBV marker levels as compared to baseline has been attained. In some embodiments, the delay period extends until the reduction in the subject's other HBV marker levels as compared to baseline is about two-fold, about three-fold, about four-fold, about five-fold, about six-fold, about seven-fold, about eight-fold, about nine-fold, about ten-fold, about eleven-fold, about twelve-fold, about thirteen fold, about fourteen fold, about fifteen-fold, about sixteen-fold, about seventeen-fold, about eighteen-fold, about nineteen-fold, about twenty-fold, about thirty-fold, about forty-fold, about fifty-fold, about sixty-fold, about seventy-fold, about eighty-fold, about ninety-fold, or about a hundred-fold, or a range constructed from any of the aforementioned values. In some embodiments, the delay period extends until there is about a 0.5 log 10 reduction, about a 1 log 10 reduction, about a 1.5 log 10 reduction, about a 2 log 20 reduction, about a 2.5 log 10 reduction, about a 3 log 10 reduction, about a 3.5 log 10 reduction, about a 4 log 10 reduction, or about a 5 log 10 reduction in the subject's other HBV marker levels as compared to baseline. Thus, in some embodiments, the methods may include regularly (such as daily) measuring a subject's other HBV marker levels, such as to use for comparison to baseline.

### Combination Therapies

In some embodiments, the first and/or second agents as described herein can be used in combination with one or more additional agent(s) for treating and/or inhibiting replication HBV and/or HDV. Additional agents include, but are not limited to, an interferon, nucleoside/nucleotide analogs, a sequence specific oligonucleotide (such as anti-sense oligonucleotide and siRNA), nucleic acid polymers (NAPs, such as nucleic acid polymers that reduce HBsAg levels including STOPS™ compounds) an entry inhibitor and/or a small molecule immunomodulator. Examples of additional agents include recombinant interferon alpha 2b, IFN-α, PEG-IFN-α-2a, lamivudine, telbivudine, adefovir dipivoxil, clevudine, entecavir, tenofovir alafenamide and tenofovir disoproxil. Examples of NAPs include, but are not limited to, REP 2139 and REP 2165. Exemplary siRNA's that can be used in combination with a compound, or pharmaceutically acceptable salt thereof, provide herein include those described in WO 2021/178885, which is hereby incorporated by reference for the purpose of describing the siRNA compounds provided therein, such as a siRNA selected from SEQ. ID. NO. 1-617 and SEQ. ID. NO. 618.

In some embodiments, a compound, or a pharmaceutically acceptable salt thereof, as described herein can be administered with one or more additional agent(s) together in a single pharmaceutical composition. In some embodiments, a compound, or a pharmaceutically acceptable salt thereof, can be administered with one or more additional agent(s) as two or more separate pharmaceutical compositions. Further, the order of administration of a compound, or a pharmaceutically acceptable salt thereof, as described herein with one or more additional agent(s) can vary.

### EXAMPLES

Some aspects of the embodiments discussed above are disclosed in further detail in the following examples, which are not in any way intended to limit the scope of the present disclosure. Those in the art will appreciate that many other embodiments also fall within the scope of the invention, as it is described herein above and in the claims.

### Example 1

In vivo compound efficacy was evaluated in adeno-associated virus-hepatitis B virus (AAV-H-BV) mice. Materials and methods are presented below in tables 1-4. Table F describes the nucleic acid sequences used for siRNA-1, an siRNA. The sequences are described using modified nucleotide annotations found in Table B.

TABLE F

| siRNA | Sequences (5′-3′) |
|---|---|
| siRNA-1 | SEQ ID NO: 2 (Sense) |
| | mC psmC psmG mU fG mU fG fC fA mC |
| | mU mU mC mG mC mU mU mC mA |
| | SEQ ID NO: 3 (Antisense) |
| | mU psfG psmA mA fG mC mG fA mA mG |
| | mU mG mC fA mC mA fC mG mGps mU psmC |

TABLE 1
Subject Details

| | | Dose Level | Dose Vol | Dose | Dose | Dose | No. |
|---|---|---|---|---|---|---|---|
| Grp | Test Article | (mg/kg/dose) | (mL/kg/ dose) | Route | Frequency | Duration | An |
| 01 | Vehicle | 0 | 5 | PO | BID | Day 0-97 | 4 |
| 02 | Compound 1 | 55 | 5 | PO | BID | Day 0-97 | 4 |
| 03 | SIRNA-1 | 5 | 5 | SC | 4 doses | Days 0, | 4 |
| | | | | | | 14, 42, 70 | |
| 04 | Compound 1 | 55 | 5 | PO | BID | Day 0-97 | 4 |
| | SIRNA-1 | 5 | 5 | SC | 4 doses | Days 0, | |
| | | | | | | 14, 42, 70 | |
| 05 | Compound 1 | 55 | 5 | PO | BID | Day 0-97 | 4 |
| | SIRNA-1 | 5 | 5 | SC | 3 doses | Days 70, | |
| | | | | | | 84, 102 | |

Abbreviations: Grp = Group; Vol = Volume; No. An = Number of Animals; PO = Oral gavage; SC = subcutaneous injections; BID = twice daily at 12 h/12 h interval.

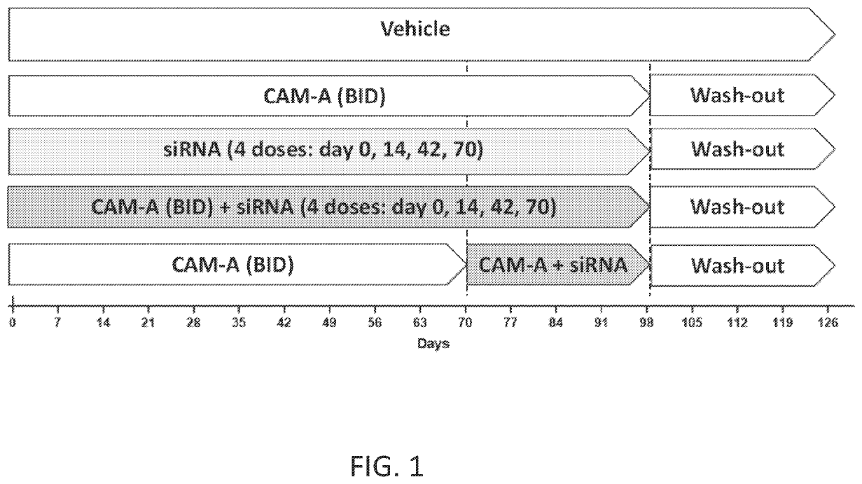

A dosing schedule is shown in FIG. 1.

TABLE 2
Subject Details

| | |
|---|---|
| Species and | Mouse: C57BL/6 |
| Strain | |
| Sex | Male |
| Approximate Age | 16-17 weeks old and transfected with AAV-HBV |
| | for about 11 weeks |
| Quantity enrolled | 20 |
| on study | |
| Environmental | Photoperiod: 12 hours light, 12 hours dark with |
| Conditions | 7:00 AM to 7:00 PM lights on (may be interrupted |
| | for study-related activities); Temperature: 21-25° C.; |
| | Relative humidity: 40%-70%. |

TABLE 3
Test Article and Dose Formulation Details

| | | Dose | Dose | | | |
|---|---|---|---|---|---|---|
| | | Level | Vol | | No. of | Prep. |
| | | (mg/kg/ | (mL/kg/ | Dose | Dose | Conc. |
| Grp | Test Article | dose) | dose) | Frequency | Days | (mg/mL) |
| 01 | Vehicle | 0 | 5 | BID | 98 | 0 |
| 02 | Compound 1 | 55 | 5 | BID | 98 | 11 |
| 03 | SIRNA-1 | 5 | 5 | 4 doses | 4 | 1 |
| 04 | Compound 1 | 55 | 6 | BID | 98 | 11 |
| | SIRNA-1 | 5 | 5 | 4 doses | 4 | 1 |
| 05 | Compound 1 | 55 | 5 | BID | 98 | 11 |
| | SIRNA-1 | 5 | 5 | 3 doses | 3 | 1 |

Abbreviations: Grp = Group; Vol = Volume; Prep = Preparation; Conc. = Concentration; QW = Once weekly; BID = Twice daily at 12 h/12 h interval.

TABLE 4
Test Article and Dose Formulation Details

| | |
|---|---|
| Dosing | Up to 102 days |
| Duration | |
| Vehicle | Vehicle for Group 01 and Compound 1: |
| | 95% PEG-400, 5% copovidone |
| | Vehicle for siRNA-1: sterile phosphate buffer |
| | saline (PBS) |
| Frequency of | Vehicle and Compound 1: Once Weekly |
| Preparation | siRNA-1: Once |
| Test Article | Compound 1: Room temperature (15-30° C.) |
| Storage | siRNA-1: Ultra Freeze (−70° C.) |
| Conditions | |
| Dose Solution | Vehicle and Compound 1: 2-8° C. |
| Storage | SIRNA-1: −20° C. |
| Conditions | |
| Dose Volume | Calculate doses based on most recent body weight. |
| Adjustment | |

Test Article Preparation: Compound 1 and siRNA-1 were provided as powder.

Preparation of vehicle (95% PEG-400, 5% copovidone, 100 mL): 5 g of copovidone (Plasdone S-630) was weighed out. 100 g of PEG400 was weighed out. Copovidone was added in small amounts to PEG400, while continuously stirring. Components were mixed until clear colorless solution was formed. The vehicle was stored at room temperature for up to one week.

Preparation of formulation of Compound 1: Required amount of test compound was weighed out. Test compound was added to appropriate amount of vehicle. Mixture was heated for 10 seconds, vortexed for 10 seconds and sonicated for 1 minute. Heating/vortexing/sonicating was repeated several times until a solution was obtained. The order of these steps can be varied.

Preparation of siRNA-1 formulation. The required volume of vehicle was added into the bottle to get a stock solution, vortexed, then put at 37° C. for half hour, and vortexed another time during incubation. It was ensured that all powder went into solution. The clarity of the solution was checked. A quick spin down was performed. The solution was diluted and OD260 was measured after dilution. Stock solution was diluted to the target concentration according to the actual concentration calculated by OD values. The OD value of each formulation was measured to get the actual concentration. The formulation was filtered using 0.22 μM PVDF filter. The formulation was aliquoted and stored at −20° C. before using.

Live Phase Parameters: Based on plasma viral markers levels and body weight, 20 mice were selected and randomized into five groups for treatment. Checks were made for dead and/or moribund animals daily. Overt changes were recorded. Body weight was measured twice weekly during Day 0-97, and once weekly during Day 98-189.

Vehicle (Group 01) and Compound 1 at 55 mg/kg/dose (Groups 02, 04 and 05) were administered by oral gavage twice daily during Day 0-97. siRNA-1 at 5 mg/kg/dose was injected subcutaneously on Days 0, 14, 42 and 70 for Groups 03 and 04, and on Days 70, 84, and 102 for Group 05.

Plasma samples of 50 μL per mouse were prepared once weekly prior to dosing during Day 0-189. The samples were used for quantitative detections of HBsAg, HBeAg, and HBV DNA. Mice were sacrificed on Day 189.

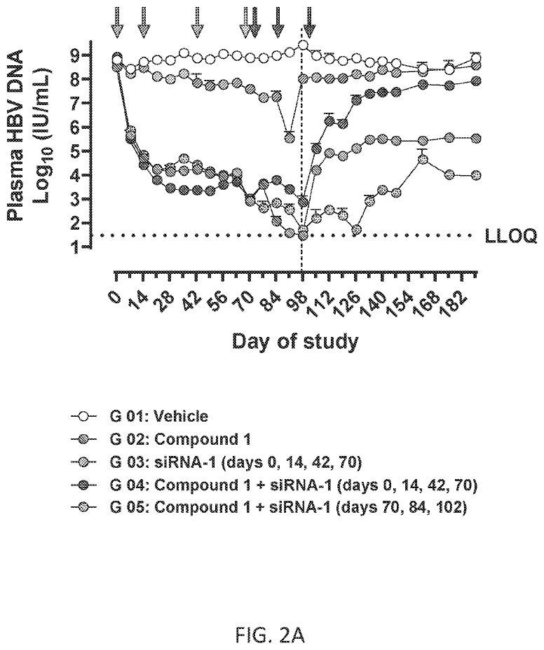

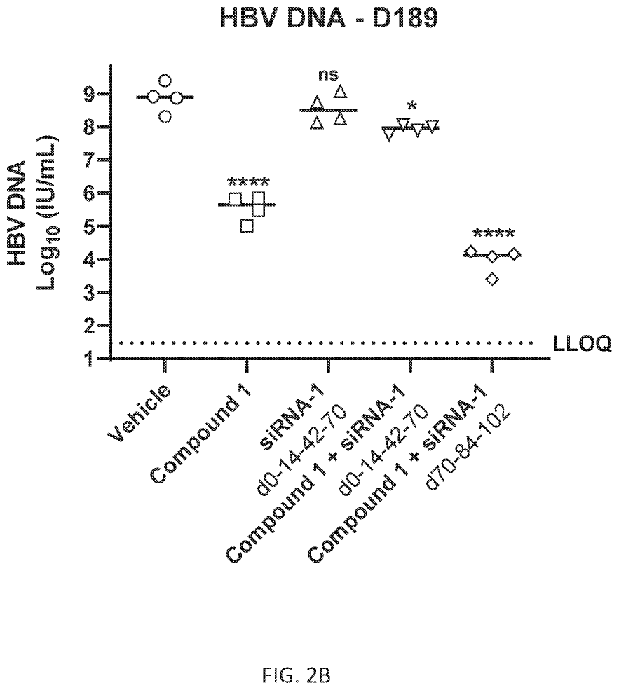

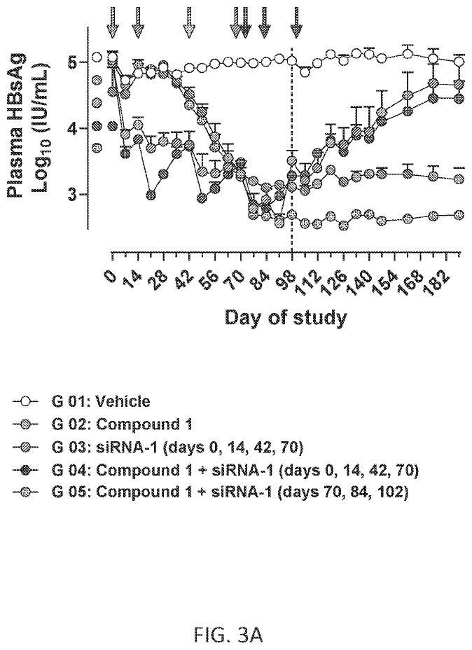

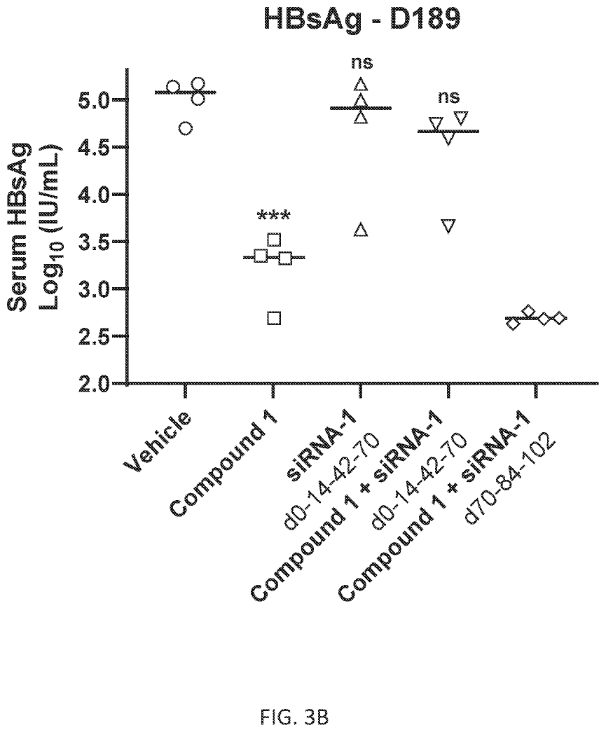

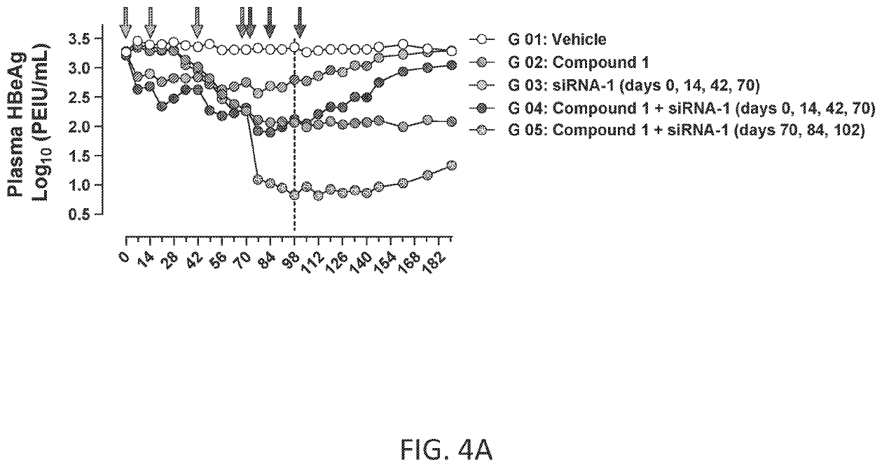

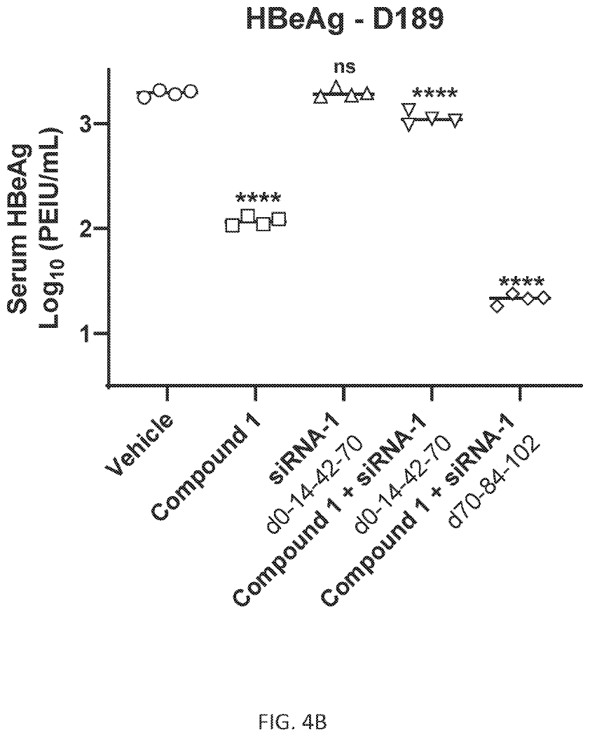

Results: Results are shown in FIGS. 2A-4B. Overall, Group 02 (CAM-A alone) is superior to Group 04 (CAM-A+siRNA co-dosing); Group 05 (siRNA add-on) is best. As shown in FIGS. 2A-2B, rebound after end of CAM-A treatment is only partial, whereas co-dosing (Group 04) rebounds close to baseline. As shown in FIGS. 3A-3B and 4A-4B, CAM-A effect is sustained, whereas rebound is observed in siRNA (Group 03) and co-dosing (Group 04) groups. As shown in FIGS. 4A-4B, a sustained HBeAg response in the add-on group (Group 05) was observed, with no seroconversion, and with only a slight rebound at the last timepoint.

### Example 2

In vivo compound efficacy was evaluated in adeno-associated virus-hepatitis B virus (AAV-HBV) mice. Materials and methods are presented below in tables 5-8. Table G describes the nucleic acid sequences used for siRNA-2, an siRNA. The sequences are described using modified nucleotide annotations found in Table B.

TABLE G

| siRNA | Sequences (5′-3′) |
|---|---|
| siRNA-2 | SEQ ID NO: 4 (Sense) |
| | mG psmU psmG mG fU mG fG fA fC mU mU |
| | mC mU mC mU mC mA mA mU |
| | SEQ ID NO: 5 (Antisense) |
| | mA psfU psmU mG mA fG mA mG mA mA mG |
| | mU mC fC mA fC mC mA mC psmA psmA |

TABLE 5
Subject Details

| | | | | Dose Vol | | | | |
|---|---|---|---|---|---|---|---|---|
| | | | | (mL/kg/ | Dose | Dose | | No. |
| Grp | Test Article | Dose Level | | dose) | Route | Freq | Dose (Day) | An |
| 01A | Vehicle | 0 | mg/kg/dose | 5 | PO | BID | 0-41 | 2 |
| 01B | Vehicle | 0 | mg/kg/dose | 5 | PO | BID | 0-97 | 3 |
| 02A | SiRNA-2 | 5 | mg/kg/dose | 5 | SC | QOW | 0-14 | 2 |
| | Vehicle | 0 | mg/kg/dose | 5 | PO | BID | 0-41 | |
| 02B | SiRNA-2 | 5 | mg/kg/dose | 5 | SC | QOW | 0, 14, 35, 49 | 3 |
| | Vehicle | 0 | mg/kg/dose | 5 | PO | BID | 0-97 | |
| 03 | Compound 7 | 50 | mg/kg/dose | 5 | PC | BID | 0-41 | 4 |
| 04 | Compound 1 | 55 | mg/kg/dose | 5 | PO | BID | 0-97 | 4 |
| 05 | SIRNA-2 | 5 | mg/kg/dose | 5 | SC | QOW | 0-14 | 4 |
| | Compound 7 | 50 | mg/kg/dose | 5 | PC | BID | 0-41 | |
| 06 | SiRNA-2 | 5 | mg/kg/dose | 5 | SC | QOW | 14, 35 | 4 |
| | Compound 7 | 50 | mg/kg/dose | 5 | PO | BID | 0-41 | |
| 07 | SIRNA-2 | 5 | mg/kg/dose | 5 | SC | QOW | 0-14 | 4 |
| | Compound 7 | 50 | mg/kg/dose | 5 | PC | BID | 14-41 | |
| 08 | SIRNA-2 | 5 | mg/kg/dose | 5 | SC | QOW | 0, 14, 35, 49 | 4 |
| | Compound 1 | 55 | mg/kg/dose | 5 | PC | BID | 0-97 | |
| 09 | SiRNA-2 | 5 | mg/kg/dose | 5 | SC | QOW | 70-84 | 4 |
| | Compound 1 | 55 | mg/kg/dose | 5 | PO | BID | 0-97 | |
| 10 | SiRNA-2 | 5 | mg/kg/dose | 5 | SC | QOW | 0, 14, 35, 49 | 4 |
| | Compound 1 | 55 | mg/kg/dose | 5 | PO | BID | 28-97 | |

Abbreviations: Grp = Group; Vol = Volume; Freq = Frequency; No. An= Number of Animals; PO = Oral gavage; SC = subcutaneous injections; QOW = once in two weeks; BID = twice daily at 12 h/12 h interval.

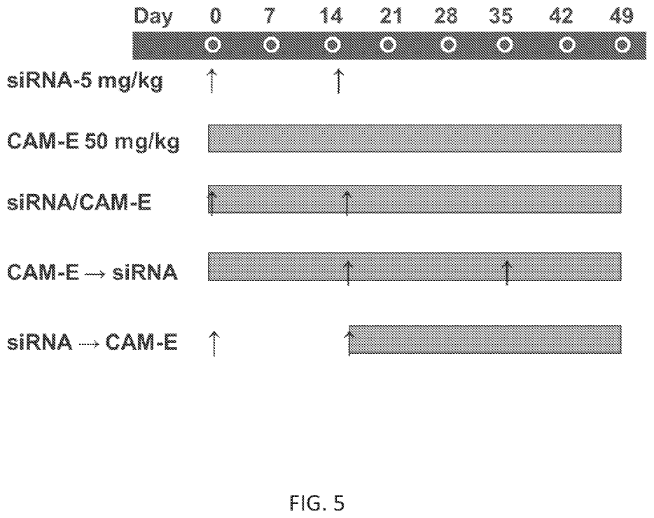

The dosing schedule for an experiment involving siRNA (siRNA-2, SEQ ID NOs: 4-5)+CAM-E (Compound 7) (Groups 01 Å, 02A, 03, 05, 06 and 07) is shown in FIG. 5.

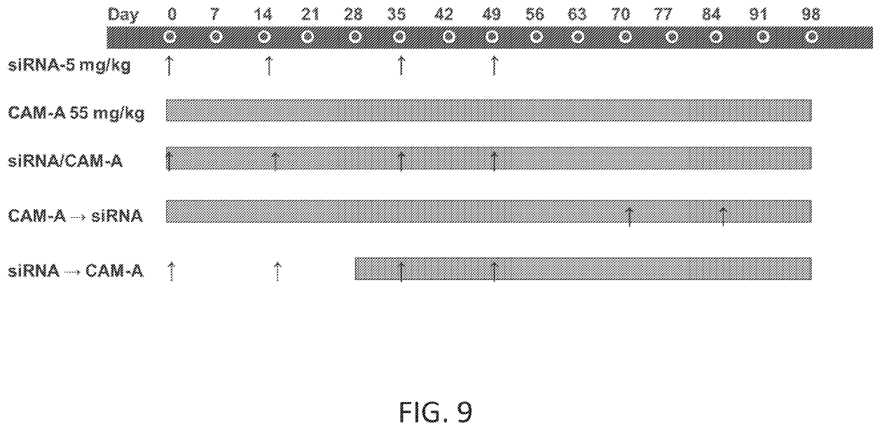

The dosing schedule for an experiment involving siRNA (siRNA-2, SEQ ID NOs: 4-5)+CAM-A (Compound 1) (Groups 011B, 02B, 04, and 08-10) is shown in FIG. 9.

TABLE 6
Subject Details

| | |
|---|---|
| Species and | Mouse: C57BL/6 |
| Strain | |
| Sex | Male |
| Approximate Age | 25-26 weeks old and transfected with AAV-HBV |
| | for about 21 weeks |
| Quantity enrolled | 50 |
| on study | |
| Environmental | Photoperiod: 12 hours light, 12 hours dark with |
| Conditions | 7:00 AM to 7:00 PM lights on (may be interrupted |
| | for study-related activities); Temperature: 21-25° C.; |
| | Relative humidity: 40%-70%. |

TABLE 7
Test Article and Dose Formulation Details

| | | | | | | No. | | |
|---|---|---|---|---|---|---|---|---|
| | | | | Dose Vol | | of | | |
| | | | | (mL/kg/ | Dose | Dose | | |
| Grp | Test Article | Dose Level | | dose) | Freq | Days | Prep. Conc. | |
| 01A | Vehicle | 0 | mg/kg/dose | 5 | BID | 42 | 0 | mg/mL |
| 01B | Vehicle | 0 | mg/kg/dose | 5 | BID | 98 | 0 | mg/mL |
| 02A | SIRNA-2 | 5 | mg/kg/dose | 5 | QOW | 2 | 1 | mg/mL |
| | Vehicle | 0 | mg/kg/dose | 5 | BID | 42 | 0 | mg/mL |
| 02B | SIRNA-2 | 5 | mg/kg/dose | 5 | QOW | 4 | 1 | mg/mL |
| | Vehicle | 0 | mg/kg/dose | 5 | BID | 98 | 0 | mg/mL |
| 03 | Compound 7 | 50 | mg/kg/dose | 5 | BID | 42 | 10 | mg/mL |
| 04 | Compound 1 | 55 | mg/kg/dose | 5 | BID | 98 | 11 | mg/mL |
| 05 | siRNA-2 | 5 | mg/kg/dose | 5 | QOW | 2 | 1 | mg/mL |
| | Compound 7 | 50 | mg/kg/dose | 5 | BID | 42 | 10 | mg/mL |
| 06 | SiRNA-2 | 5 | mg/kg/dose | 5 | QOW | 2 | 1 | mg/mL |
| | Compound 7 | 50 | mg/kg/dose | 5 | BID | 42 | 10 | mg/mL |
| 07 | siRNA-2 | 5 | mg/kg/dose | 5 | QOW | 2 | 1 | mg/mL |
| | Compound 7 | 50 | mg/kg/dose | 5 | BID | 28 | 10 | mg/mL |
| 08 | SiRNA-2 | 5 | mg/kg/dose | 5 | QOW | 4 | 1 | mg/mL |
| | Compound 1 | 55 | mg/kg/dose | 5 | BID | 98 | 11 | mg/mL |
| 09 | siRNA-2 | 5 | mg/kg/dose | 5 | QOW | 2 | 1 | mg/mL |
| | Compound 1 | 55 | mg/kg/dose | 5 | BID | 98 | 11 | mg/mL |
| 10 | siRNA-2 | 5 | mg/kg/dose | 5 | QOW | 4 | 1 | mg/mL |
| | Compound 1 | 55 | mg/kg/dose | 5 | BID | 70 | 11 | mg/mL |

Abbreviations: Grp = Group; Vol = Volume; Freq = Frequency; Conc. = Concentration; QW = once weekly; BID = twice daily at 12 h/12 h interval.

TABLE 8
Test Article and Dose Formulation Details

| | |
|---|---|
| Dosing | Up to 98 days |
| Duration | |
| Vehicle | Vehicle for dosing in Groups 01A, 01B, 02A, and |
| | 02B, and vehicle for preparing Compound 7 and |
| | Compound 1 formulations: 95% PEG-400, 5% |
| | copovidone Vehicle for siRNA-2: sterile phosphate |
| | buffer saline (PBS) |
| Frequency of | Vehicle and Compound 1 and Compound 7: |
| Preparation | Once Weekly SiRNA-2: Once |
| Test Article | Compound 1: 15-30° C. |
| Storage | Compound 7 and siRNA-2: −20° C. |
| Conditions | |
| Dose Solution | Vehicle, Compound 7, and Compound 1: 2-8° C. |
| Storage | SIRNA-2: −20° C. |
| Conditions | |
| Dose Volume | Calculate doses based on most recent body weight. |
| Adjustment | |

Compound 1, Compound 7, and siRNA-2 were provided as powder and formulated by Labcorp.

The vehicle (95% PEG-400, 5% copovidone, 100 mL) was prepared with the following steps. 5 g of copovidone (Plasdone S-630) was weighed out. 100 g of PEG400 was weighed out. Copovidone was added in small amounts to PEG400, while continuously stirring. Components were mixed until clear colorless solution was formed. The vehicle was stored at room temperature for up to one week.

Formulations of Compound 1 and Compound 7 were prepared with the following steps. Required amount of test compound was weighed out. Test compound was added to appropriate amount of vehicle. The mixture was heated for 10 seconds, vortexed for 10 seconds and sonicated for 1 minute. Heating/vortexing/sonicating was repeated several times until a solution was obtained. The order of these steps can be varied.

Formulation of siRNA-2 was prepared with the following steps. The required volume of vehicle was added into the bottle to get a stock solution, vortexed, then put at 37° C. for half hour, and vortexed another time during incubation. It was ensured that all the powder got into solution. The clarity of the solution was checked. If it appeared turbid or precipitated, it was left at 50° C. for half an hour. A quick spin down was performed, then the solution was diluted and OD260 was measured after dilution. The stock solution was diluted to the target concentration according to the actual concentration calculated by OD values. The OD value of each formulation was measured to get the actual concentration. The formulation was filtered using 0.22 μm PVDF filter. The formulation was aliquoted, and stored at −20° C. before using.

Pre-treatment parameters: Animal health was monitored during model pre-treatment phase. Body weight was measured on Predose Day 0. Mice were bled for serum preparation (10 μL per mouse) on Predose Day 0. Samples were stored at −70° C. before transferred for quantitative detections of HBsAg, HBeAg, and HBV DNA.

Live Phase Parameters: Based on serum HBsAg, HBeAg, and HBV DNA levels and body weight on Predose Day 0, animals were assigned using an Excel tool to cages. Low, medium, and high HBV titer values were spread to equalize group mean values across groups.

Cages were checked for dead and/or moribund animals daily, and overt changes were recorded. Body weight was measured twice weekly during Day 0-97. Additional body weight measurement was applied based on Study Director's justification.

Vehicle was administered at 5 mL/kg/dose by oral gavage twice daily (with 12-hour interval) during Day 0-41 in Groups 01 A and 02A, and during Day 0-97 in Group 1B. siRNA-2 at 5 mg/kg/dose was administered at 5 mL/kg/dose by subcutaneous injection on Day 0 and Day 14 in Groups 02A, 05, and 07, and on Days 0, 14, 35, and 49 in Groups 02B, 08, and 10, on Day 14 and 35 in Group 06, and on 70 and 84 in Group 09.

Compound 7 at 50 mg/kg/dose was administered at 5 mL/kg/dose by oral gavage twice daily (with 12-hour interval) during Day 0-41 in Groups 03, 05, and 06, and during Day 14-41 in Group 07.

Compound 1 at 55 mg/kg/dose was administered by oral gavage twice daily (with 12-hour interval) during Day 0-97 in Groups 04, 08, and 09, and during Day 28-97 in Group 10.

Mice were bled to prepare 25 μL of serum per mouse once weekly during Day 0-98. The samples were stored at −20° C. before being transferred for HBsAg, HBeAg, HBV DNA, and ALT detections.

Mice were sacrificed in Groups 01A, 02A, 03, 05, 06 and 07 on Day 42, and the mice in Groups 01B, 02B, 04, and 08-10 were sacrificed on Day 98.

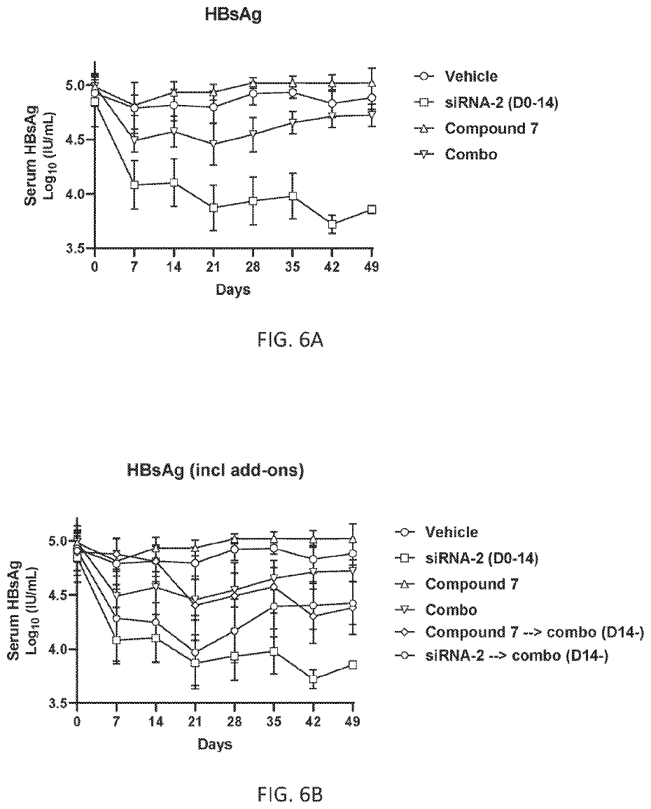

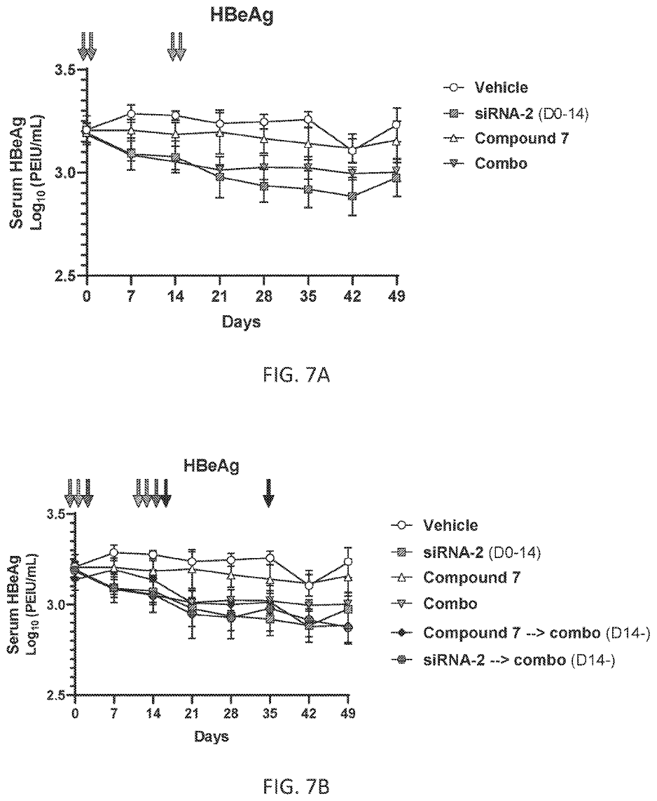

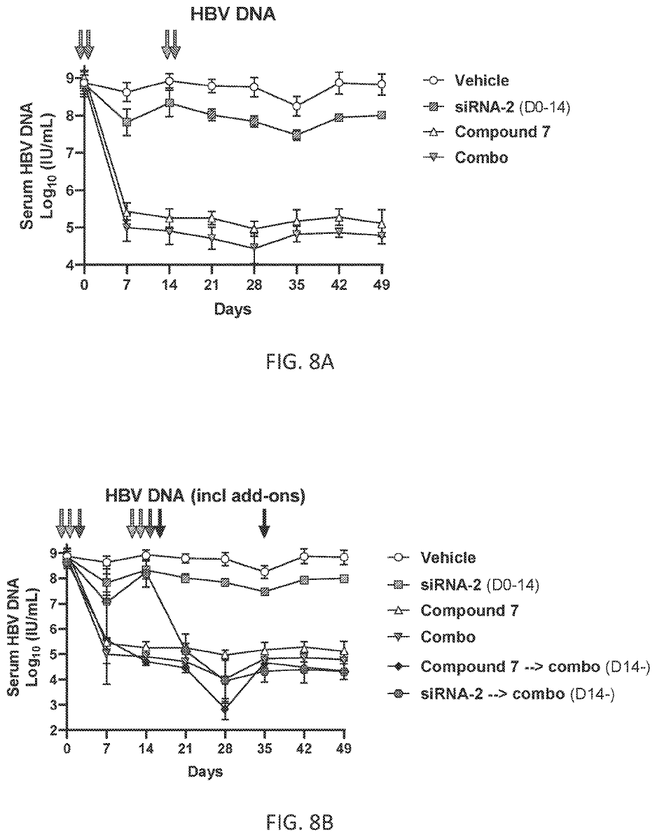

Results: FIGS. 6A-6B show the antagonism on HBsAg reductions when CAM-E Compound 7 was combined with an siRNA (combo). Sequential dosing regimens (Compound 7→combo (D14-) and siRNA-2→combo (D14-)) show a slight improvement (less antagonism). Other sequential regimens may be able to fully overcome the antagonism. HBeAg readouts shown in FIGS. 7A-7B show a more limited antagonism of combinations on this parameter, possibly due to the smaller magnitude of the HBeAg reduction. No obvious antagonism or added benefit of sequential regimens was noted on the HBV DNA read-out as shown in FIGS. 8A and 8B.

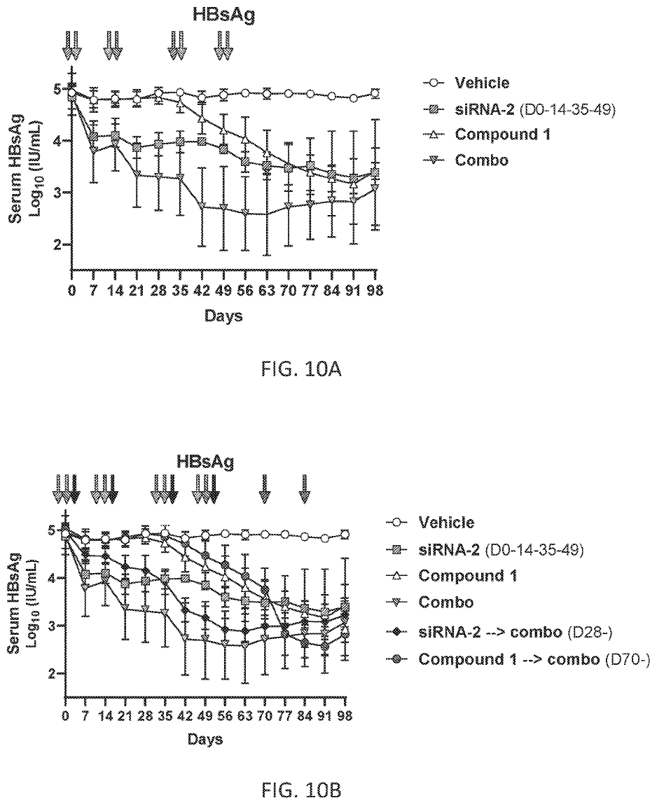

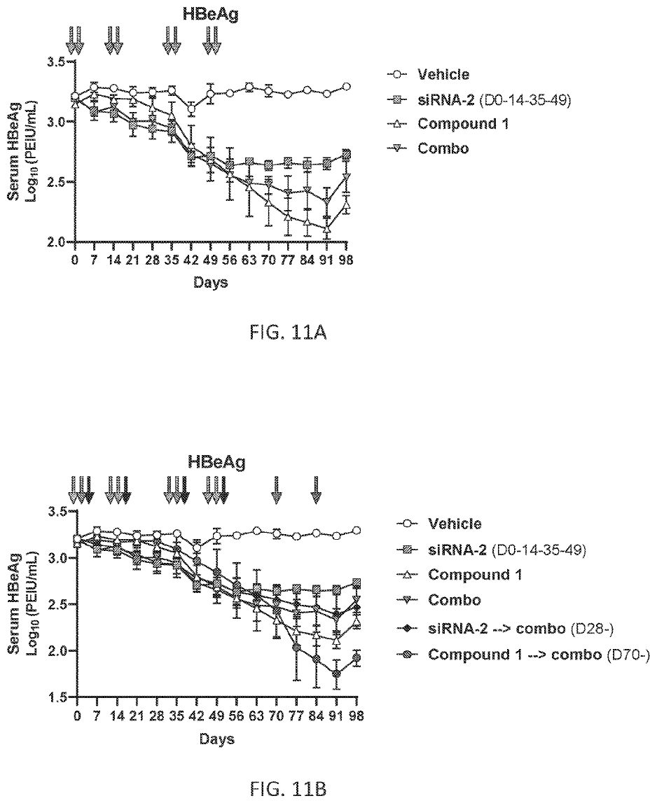

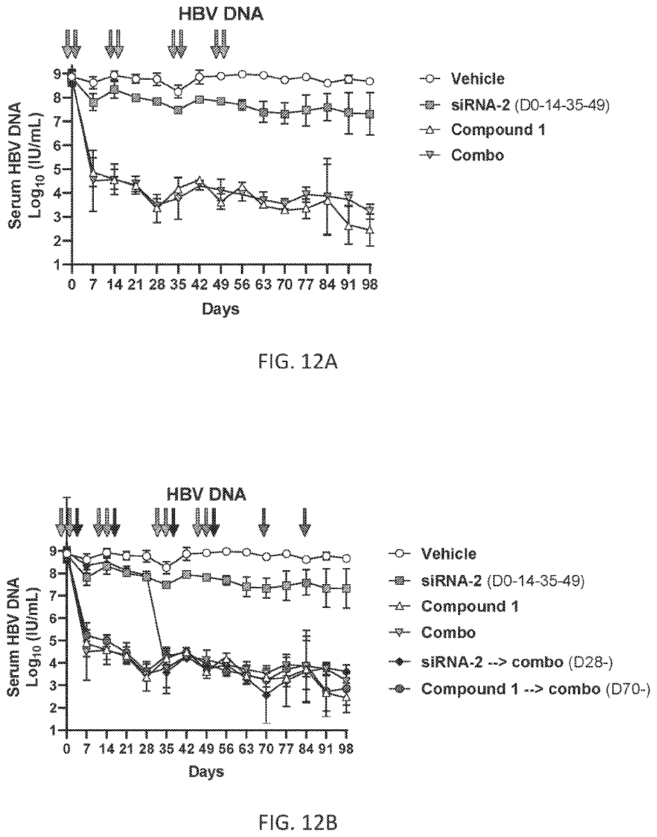

As seen in FIGS. 10A-10B, the combination of CAM-A+siRNA (combo) showed no antagonism in reduction of HBsAg measurements as compared to siRNA alone (siRNA-2 (DO-14-35-49)) and even led to an additional decline in the first half of the study. Moreso, the addition of CAM-A to the siRNA treatment at a later time point (siRNA-2→combo (D28-)) also showed a potentiation of the siRNA efficacy. The most favorable regimen consisted of adding the siRNA to the CAM-A (Compound 1→combo (D)70-)), which led to an additional reduction in serum HBsAg levels from day 77 on. Similar trends were observed for HBeAg (FIGS. 11A-11B). For the HBV DNA read-out (FIGS. 12A-12B), differences between regimens were limited, likely due to the deep HBV DNA reduction already induced by CAM-A monotherapy.

### Example 3

In the following example, initial treatment with an siRNA, followed by CAM add-on, was tested in vivo in mice.

The study design is described below in Table 9.

TABLE 9
Study Design

| | Day 0-98 | |
|---|---|---|
| Group | [HBsAg | CAM add-on |
| [n = 4 each] | nadir - day 63] | [day 63-98] |
| 1 | Vehicle SC QW | Vehicle PO |
| 2 | | CAM-E (Compound 6) 30 mpk BID |
| 3 | | CAM-A (Compound 2) 30 mpk BID |
| 4 | SiRNA-2 | Vehicle PO |
| 5 | 10 mpk SC QW | CAM-E (Compound 6) 30 mpk BID |
| 6 | | CAM-A (Compound 2) 30 mpk BID |

QW dosing of siRNA-2 was continued until the end of the study (day 98). HBV DNA, HBsAg, and HBeAg were measured weekly through day 98.

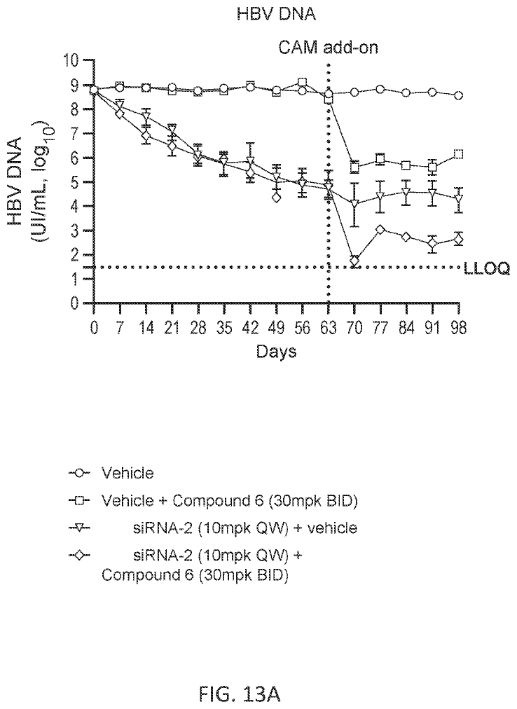

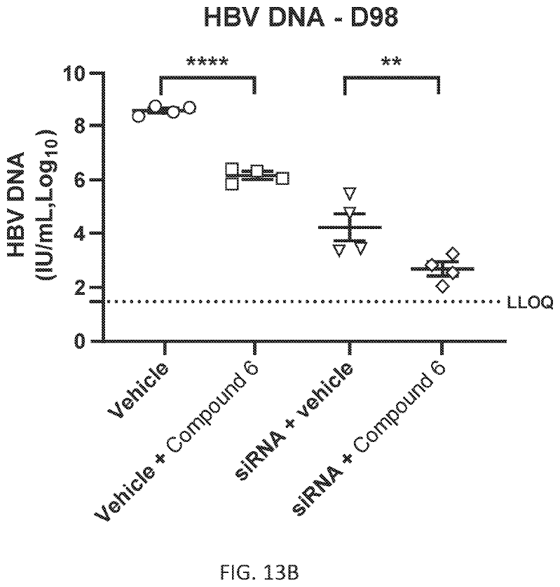

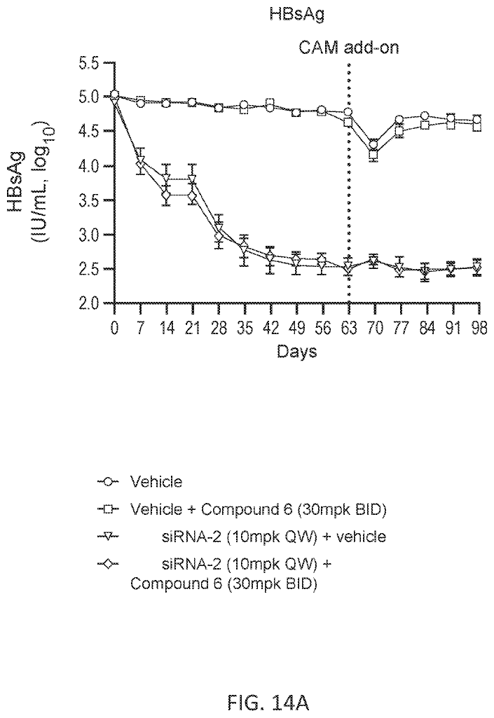

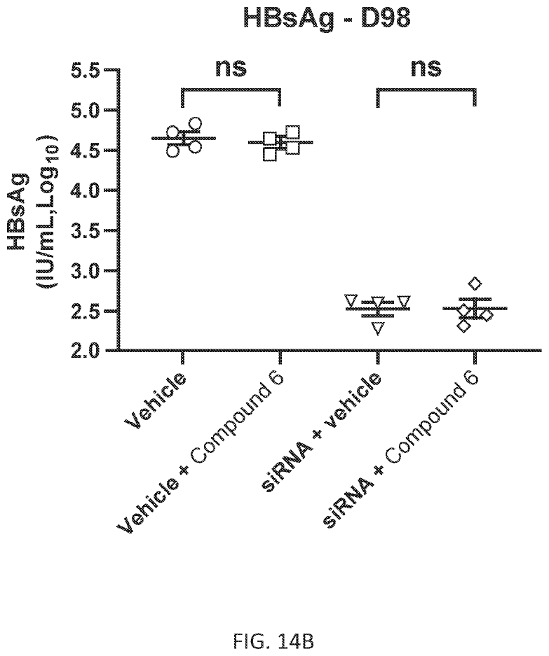

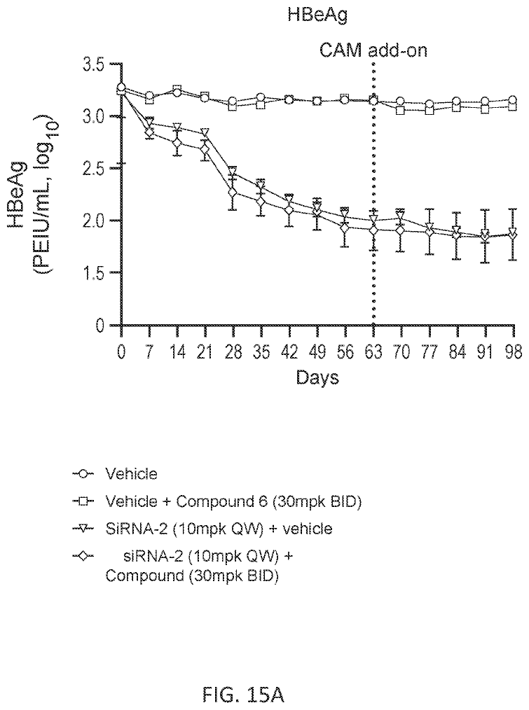

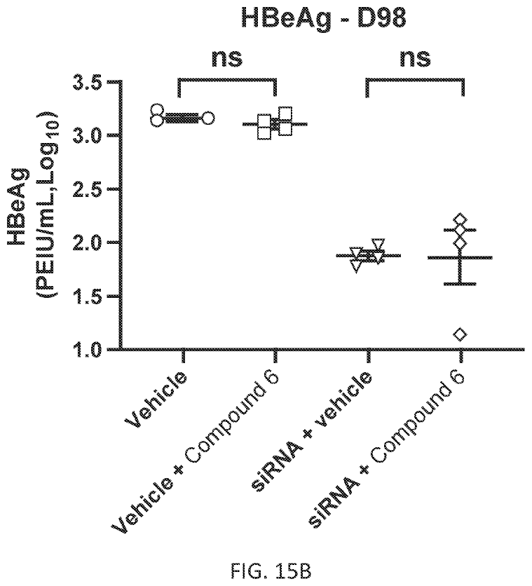

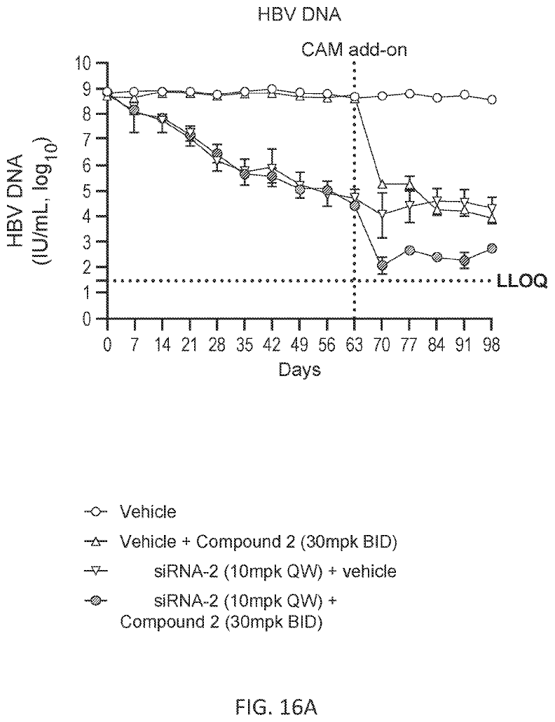

Results: As shown in FIGS. 13A and 13B, administration of siRNA followed by CAM-E (Compound 6) resulted in a good reduction of HBV DNA, even greater than the siRNA-only group (Group 4). As depicted in FIGS. 14A, 14B, 15A, and 15B, by day 98, HBsAg/HBeAg reduction was greater for siRNA+CAM-E add-on compared to CAM-E alone, and no antagonistic effect had been observed for siRNA+CAM-E add-on (Group 5) compared to siRNA only, unlike the antagonistic effect observed in the co-dosing groups in Example 2.

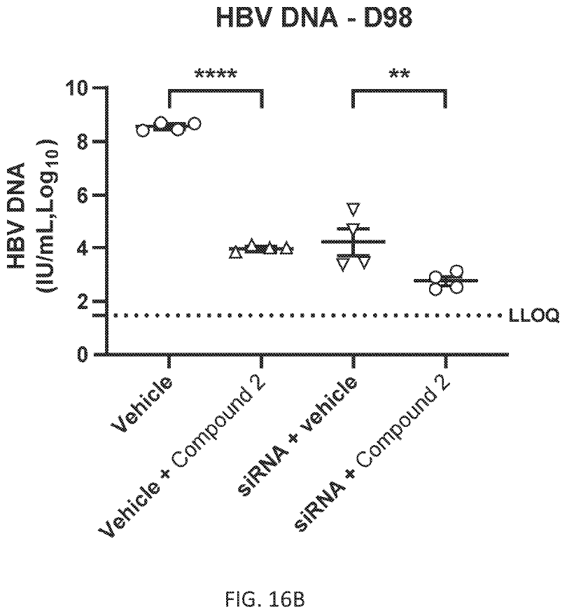

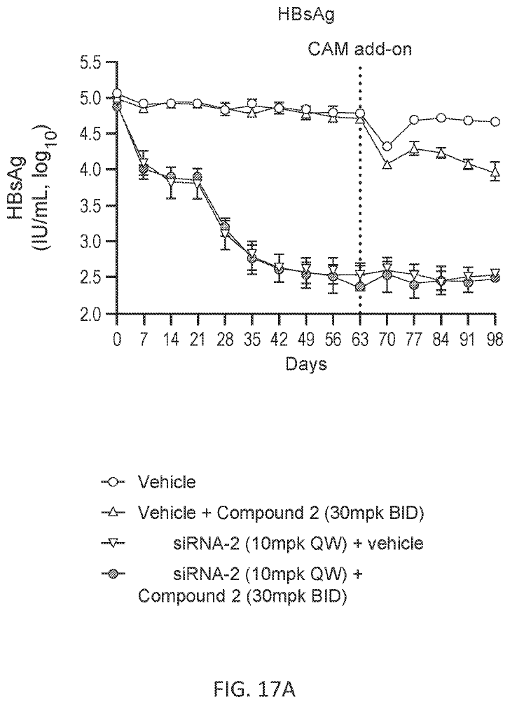

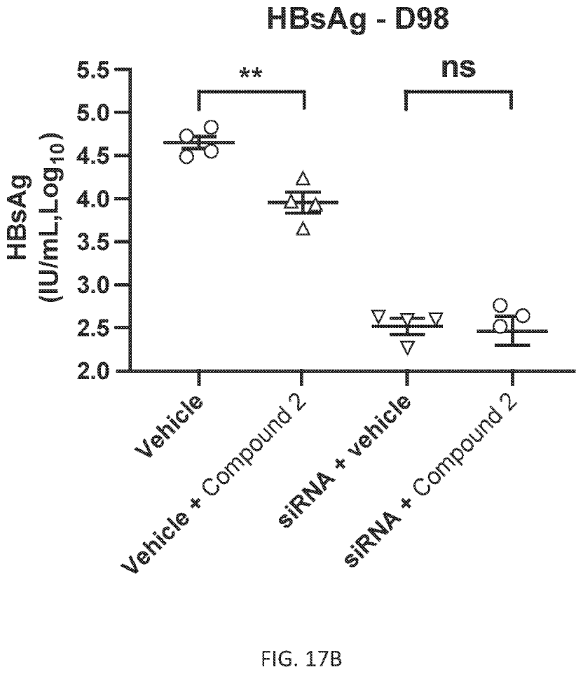

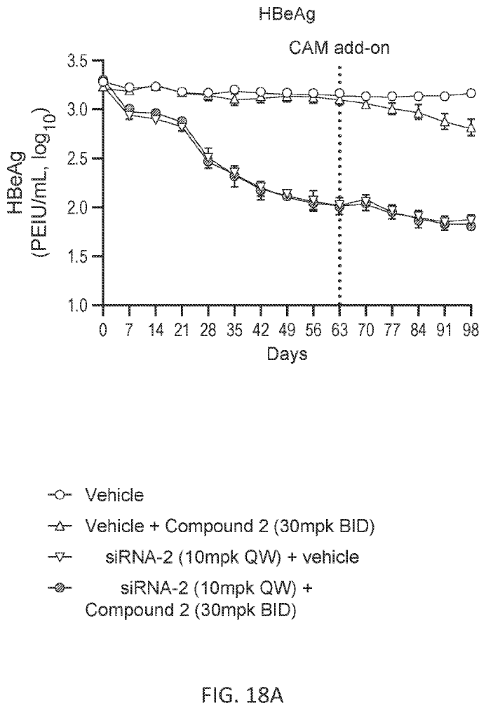

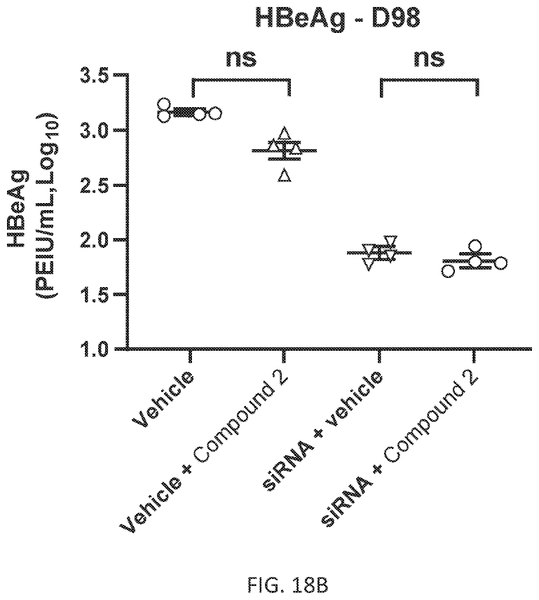

As shown in FIGS. 16A and 16B, the CAM-A (Compound 2)-only group resulted in a good reduction of HBV DNA, while the siRNA+CAM-A add-on group (Group 6) resulted in an even greater reduction of HBV DNA. As depicted in FIGS. 17A, 17B, 18A, and 18B, by day 98, HBsAg/HBeAg reduction was greater for siRNA+CAM-A add-on compared to CAM-A alone, and no antagonistic effect had been observed for siRNA+CAM-A add-on (Group 6) compared to siRNA only.

Discussion: The above results suggest that sequential, delayed dosing could unexpectedly be a solution for any antagonism observed for simultaneous dosing, such as seen in the co-dosing groups in Example 2.

### Other Considerations

Headings are included herein for reference and to aid in locating various sections. These headings are not intended to limit the scope of the concepts described with respect thereto. Such concepts may have applicability throughout the entire specification.

The entirety of each patent, patent application, publication and document referenced herein hereby is incorporated by reference. Citation of the above patents, patent applications, publications and documents is not an admission that any of the foregoing is pertinent prior art, nor does it constitute any admission as to the contents or date of these publications or documents. Their citation is not an indication of a search for relevant disclosures. All statements regarding the date(s) or contents of the documents is based on available information and is not an admission as to their accuracy or correctness.

In the foregoing description, specific details are given to provide a thorough understanding of the examples. However, it will be understood by one of ordinary skill in the art that the examples may be practiced without these specific details.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the claims.

The methods disclosed herein comprise one or more steps or actions for achieving the described method. The method steps and/or actions may be interchanged with one another without departing from the scope of the present disclosure. In other words, unless a specific order of steps or actions is required for proper operation of the method that is being described, the order and/or use of specific steps and/or actions may be modified without departing from the scope of the present disclosure.

In at least some of the described embodiments, one or more elements used in an embodiment can interchangeably be used in another embodiment unless such a replacement is not technically feasible. It will be appreciated by those skilled in the art that various other omissions, additions and modifications may be made to the methods and structures described above without departing from the scope of the claimed subject matter. All such modifications and changes are intended to fall within the scope of the subject matter, as defined by the appended claims.

With respect to the use of substantially any plural or singular terms herein, those having skill in the art can translate from the plural to the singular or from the singular to the plural as is appropriate to the context or application. The various singular/plural permutations may be expressly set forth herein for sake of clarity.

The embodiments illustratively described herein suitably may be practiced in the absence of any element(s) not specifically disclosed herein. Thus, for example, in each instance herein any of the terms “comprising,” “consisting essentially of,” and “consisting of” may be replaced with either of the other two terms. The terms and expressions which have been employed are used as terms of description and not of limitation, and use of such terms and expressions do not exclude any equivalents of the features shown and described or portions thereof, and various modifications are possible within the scope of the technology claimed. The term “a” or “an” can refer to one of or a plurality of the elements it modifies (for example, “a reagent” can mean one or more reagents) unless it is contextually clear either one of the elements or more than one of the elements is described. The term “about” or “approximately” as used herein refers to a value within 10% of the underlying parameter (i.e., plus or minus 100%), and use of the term “about” or “approximately” at the beginning of a string of values modifies each of the values (i.e., “about 1, 2 and 3” refers to about 1, about 2 and about 3). For example, a weight of “about 100 grams” can include weights between 90 grams and 110 grams. Further, when a listing of values is described herein (for example, about 50%, 60%, 70%, 80%, 85% or 86%) the listing includes all intermediate and fractional values thereof (for example, 54%, 85.4%). Thus, it should be understood that although the present technology has been specifically disclosed by representative embodiments and optional features, modification and variation of the concepts herein disclosed may be resorted to by those skilled in the art, and such modifications and variations are considered within the scope of the embodiments.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

All published patents, published patent applications and other non-patent publications referenced and described herein are each incorporated herein by reference in its entirety. Furthermore, although the foregoing has been described in some detail by way of illustrations and examples for purposes of clarity and understanding, it will be understood by those of skill in the art that numerous and various modifications can be made without departing from the spirit of the present disclosure. Therefore, it should be clearly understood that the forms disclosed herein are illustrative only and are not intended to limit the scope of the present disclosure, but rather to also cover all modification and alternatives coming with the true scope and spirit of the disclosure.

What is claimed is:

1. A method of treating hepatitis B viral (HBV) and/or hepatitis D viral (HDV) infection in a subject in need thereof comprising:
administering an effective amount of a first agent selected from the group consisting of (i) an HBV surface antigen (HBsAg) reducing agent, or a pharmaceutically acceptable salt thereof, and (ii) a capsid assembly modulator (CAM), or a pharmaceutically acceptable salt thereof, to the subject, followed by
administering an effective amount of a second agent selected from the group consisting of (i) a CAM, or a pharmaceutically acceptable salt thereof, and (ii) an HBsAg reducing agent, or a pharmaceutically acceptable salt thereof, to the subject,
wherein when the first agent is an HBsAg reducing agent, or a pharmaceutically acceptable salt thereof, the second agent is a CAM, or a pharmaceutically acceptable salt thereof; and wherein when the first agent is a CAM, or a pharmaceutically acceptable salt thereof, the second agent is an HBsAg reducing agent, or a pharmaceutically acceptable salt thereof; and
wherein initial administration of the second agent is after a delay period following initial administration of the first agent.

2. The method of claim 1, wherein the method is a method of treating an HBV infection.

3. The method of claim 1, wherein the method is a method of treating an HDV infection.

4. The method of any one of claims 1-3, wherein the first agent is an HBsAg reducing agent or a pharmaceutically acceptable salt thereof; and wherein the second agent is a CAM, or a pharmaceutically acceptable salt thereof.

5. The method of any one of claims 1-3, wherein the first agent is a CAM, or a pharmaceutically acceptable salt thereof; and wherein the second agent is an HBsAg reducing agent, or a pharmaceutically acceptable salt thereof.

6. The method of any one of claims 1-5, wherein the CAM is a Class A CAM (CAM-A), or a pharmaceutically acceptable salt thereof.

7. The method of any one of claims 1-5, wherein the CAM is a Class E CAM (CAM-E), or a pharmaceutically acceptable salt thereof.

8. The method of any one of claims 1-7, wherein the HBsAg reducing agent is a small interfering RNA (siRNA) or an antisense oligonucleotide (ASO).

9. The method of claim 6, wherein the CAM is a fused pyrazole compound, a fused pyrimidone compound, or a pyrrole compound, or a pharmaceutically acceptable salt of any of the foregoing.

10. The method of any one of claims 1-9, wherein the CAM is selected from the group consisting of;
N-methyl-4-[(11R)-12-[4-bromo-3-(trifluoromethyl)benzoyl]-5-(cyclopropylmethyl)-11-methyl-8-oxo-2,3,7,12-tetrazatricyclo[7.4.0.0\hat{ }2,6]trideca-1(9),3,5-trien-7-yl]benzamide (Compound 1):
4-((R)-7-(4-bromo-3-(trifluoromethyl)benzoyl)-2-(((S)-but-3-en-2-yl)amino)-6-methyl-4-oxo-5,6,7,8-tetrahydropyrido[3,4-d]pyrimidin-3(4H)-yl)-N-methylbenzamide (Compound 2);
N-[4-fluoro-3-(trifluoromethyl)phenyl]-1,3,5-trimethyl-4-[2-oxo-2-[[(1S)-1-(hydroxymethyl)-1-methyl-prop-2-ynyl]amino]acetyl]pyrrole-2-carboxamide (Compound 3);
[[2-[5-[(3-cyano-4-fluoro-phenyl)carbamoyl]-1,2,4-trimethyl-pyrrol-3-yl]-2-oxo-acetyl]-(3-ethynyloxetan-3-yl)amino]methyl dihydrogen phosphate (Compound 4);
N-(3-cyano-4-fluoro-phenyl)-4-[2-[(3-ethynyloxetan-3-yl)amino]-2-oxo-acetyl]-1,3,5-trimethyl-pyrrole-2-carboxamide (Compound 5);
(S)-2-(2-(5-((4-fluoro-3-(trifluoromethyl)phenyl)carbamoyl)-1,2,4-trimethyl-1H-pyrrol-3-yl)-2-oxoacetamido)-2-methylbut-3-yn-1-yl dihydrogen phosphate (Compound 6);
(R)-N-(2-chloropyridin-4-yl)-3-fluoro-1-methyl-4-(N-(1,1,1-trifluoropropan-2-yl)sulfamoyl)-1H-pyrrole-2-carboxamide (Compound 7);
BAY 41-4109 (Compound 8);
GLS4 (Compound 9);
NVR 3-778 (Compound 10);
RG7907 (Compound 11);
AB1-H0731 (Compound 12);
ABI-3773 (Compound 13);
AB1-4334 (Compound 14);
GLP-26 (Compound 15);
KL-060332 (Compound 16):
AB-836 (Compound 17);
VNRX-9945 (Compound 18);
(R)-N-(3-cyano-4-fluorophenyl)-1-methyl-4-(N-(1,1,1-trifluoropropan-2-yl)sulfamoyl)-1H-pyrrole-2-carboxamide (Compound 19);
JNJ-64530440 (Compound 20);
EDP-514 (Compound 21);
(S)-N-(3-cyano-4-fluorophenyl)-7-methyl-3-vinyl-3,4-dihydro-2H,7H-pyrrolo[3,4-b][1,4,5]oxathiazepine-6-carboxamide 1,1-dioxide (Compound 22);
ZM-H1505R (Compound 23);
(R)-7-(4-bromo-3-chlorobenzoyl)-2-(4-cyclopropoxyphenyl)-6-methyl-3-oxo-N-(2-(pyrimidin-4-yl)benzyl)-2,3,5,6,7,8-hexahydroimidazo[1,5-a]pyrazine-1-carboxamide (Compound 24);
(6R)-7-[4-bromo-3-(trifluoromethyl)benzoyl]-2-(3,5-dimethylpyrazol-1-yl)-6-methyl-3-(3-methylimidazo[4,5-b]pyridin-6-yl)-6,8-dihydro-5H-pyrido[3,4-d]pyrimidin-4-one (Compound 25);
4-[5-benzyl-12-(4-bromo-3-chloro-benzoyl)-8-oxo-2,3,7,12-tetrazatricyclo[7.4.0.0\hat{ }2,6]trideca-1(9),3,5-trien-7-yl]-N-methyl-benzamide (Compound 26); and
(6R)-7-[4-bromo-3-(trifluoromethyl)benzoyl]-2-(3,5-dimethylpyrazol-1-yl)-3-[4-[(2S)-2-hydroxypropoxy]phenyl]-6-methyl-6,8-dihydro-5H-pyrido[3,4-d]pyrimidin-4-one (Compound 27), or a pharmaceutically acceptable salt of any of the foregoing.

11. The method of any one of claims 1-10, wherein the HBsAg reducing agent is an siRNA.

12. The method of claim 11, wherein the siRNA is a compound selected from the group consisting of RG6346 (Roche/Dicerna), ARC-520 (Arrowhead), ARC-521 (Arrowhead), ALN-HBV (Alnylam/VIR), VIR-2218 (Alnylam/VIR), AB-729 (Arbutus), and JNJ3989 (Arrowhead/JNJ).

13. The method of claim 11, wherein the siRNA has a nucleic acid sequence as set forth in SEQ ID NO: 2, SEQ ID NO: 3, SEQ ID NO: 4, SEQ ID NO: 5, SEQ ID NO: 6, or SEQ ID NO: 7.

14. The method of any one of claims 1-10, wherein the HBsAg reducing agent is an ASO.

15. The method of claim 14, wherein the ASO is a compound selected from the group consisting of GSK-404 (Isis/GlaxoSmithKline), GSK-836 (Isis/GlaxoSmithKline), and RG6004 (Roche).

16. The method of claim 14, wherein the ASO has a nucleic acid sequence as set forth in SEQ ID NO: 1.

17. The method of any one of claims 1-16, wherein initial administration of the second agent occurs after HBsAg levels have been reduced in the subject by administration of the first agent.

18. The method of claim 17, wherein initial administration of the second agent occurs after HBsAg levels have been reduced to a nadir in the subject by administration of the first agent.

19. The method of claim 18, wherein the nadir comprises a period of at least one week including at least one additional dose of the first agent, wherein the at least one additional dose results in no statistically significant reduction of HBsAg levels.

20. The method of any one of claims 1-19, wherein initial administration of the second agent occurs after the first agent has been continuously administered for at least one month.

21. The method of any of claims 1-20, wherein the delay period is greater than approximately 50 days.

22. The method of any of claims 1-21, wherein the delay period is greater than approximately 2 months.

23. The method of any one of claims 1-22, wherein the delay period is between approximately 21 days and approximately 168 days.

24. The method of any one of claims 1-23, wherein the delay period is between approximately 28 days and approximately 91 days.

25. The method of any one of claims 1-24, wherein the delay period is between approximately 8 weeks and approximately 18 weeks.

26. The method of any one of claims 1-25, wherein the delay period is approximately 50 days.

27. The method of any one of claims 1-26, wherein the first agent is administered at least three times at regular intervals, before administration of the second agent.

28. The method of any one of claims 1-27, wherein the second agent is Compound 1, the first agent is an siRNA having the nucleic acid sequences set forth in SEQ ID NO: 2 and SEQ ID NO: 3, and wherein the delay period comprises 50 days.

29. The method of any one of claims 1-28, wherein the delay period is determined based on measurement of the subject's plasma HBsAg levels.

30. The method of any one of claims 1-29, wherein the delay period extends until the subject's HBsAg levels are reduced as compared to baseline HBsAg levels.

31. The method of any one of claims 1-30, wherein the delay period extends until the subject's plasma HBsAg levels reach a nadir.

32. The method of any one of claims 1-31, wherein (a) the first agent is Compound 4, or pharmaceutically acceptable salt thereof, and the second agent is an siRNA having the nucleic acid sequences set forth in SEQ ID NO: 6 and SEQ ID NO: 7; or wherein (b) the first agent is an siRNA having the nucleic acid sequences set forth in SEQ ID NO: 6 and SEQ ID NO: 7, and the second agent is Compound 4, or a pharmaceutically acceptable salt thereof.

33. The method of any one of claims 1-32, further comprising administering an additional agent selected from the group consisting of an interferon, a nucleoside analog, a nucleotide analog, a sequence specific oligonucleotide, a nucleic acid polymer, an entry inhibitor and a small molecule immunomodulator, or a pharmaceutically acceptable salt of any of the foregoing.

34. The method of claim 33, wherein the additional agent is selected from the group consisting of recombinant interferon alpha 2b, IFN-α, PEG-IFN-α-2a, lamivudine, telbivudine, adefovir dipivoxil, clevudine, entecavir, tenofovir alafenamide, and tenofovir disoproxil and an additional siRNA, or a pharmaceutically acceptable salt of any of the foregoing.

35. A method of maintaining low plasma HBsAg levels in a subject having an HBV and/or an HDV infection, comprising:
administering an effective amount of a first agent selected from the group consisting of (i) an HBV surface antigen (HBsAg) reducing agent, or a pharmaceutically acceptable salt thereof, and (ii) a capsid assembly modulator (CAM), or a pharmaceutically acceptable salt thereof, to the subject, followed by
administering an effective amount of a second agent selected from the group consisting of (i) a CAM, or a pharmaceutically acceptable salt thereof, and (ii) an HBsAg reducing agent, or a pharmaceutically acceptable salt thereof, to the subject,
wherein when the first agent is an HBsAg reducing agent, or a pharmaceutically acceptable salt thereof, the second agent is a CAM, or a pharmaceutically acceptable salt thereof; and wherein when the first agent is a CAM, or a pharmaceutically acceptable salt thereof, the second agent is an HBsAg reducing agent, or a pharmaceutically acceptable salt thereof;
wherein initial administration of the second agent is after a delay period following initial administration of the first agent.

36. The method of claim 35, wherein the method is a method of treating an HBV infection.

37. The method of claim 35, wherein the method is a method of treating an HDV infection.

38. The method of any one of claims 35-37, wherein the first agent is an HBsAg reducing agent or a pharmaceutically acceptable salt thereof; and wherein the second agent is a CAM, or a pharmaceutically acceptable salt thereof.

39. The method of any one of claims 35-37, wherein the first agent is a CAM, or a pharmaceutically acceptable salt thereof, and wherein the second agent is an HBsAg reducing agent, or a pharmaceutically acceptable salt thereof.

40. The method of claim 36, wherein the first agent is a short interfering RNA (siRNA), and wherein the second agent is a Class A capsid assembly modulator (CAM-A) or a Class E capsid assembly modulator (CAM-E), or a pharmaceutically acceptable salt of any of the foregoing.

41. The method of claim 39, wherein the first agent is a Class A capsid assembly modulator (CAM-A) or a Class E capsid assembly modulator (CAM-E), or a pharmaceutically acceptable salt of any of the foregoing, and wherein the second agent is a short interfering RNA (siRNA).

42. The method of any one of claims 35-41, wherein (a) the first agent is Compound 4, or pharmaceutically acceptable salt thereof, and the second agent is an siRNA having the nucleic acid sequences set forth in SEQ ID NO: 6 and SEQ ID NO: 7; or wherein (b) the first agent is an siRNA having the nucleic acid sequences set forth in SEQ ID NO: 6 and SEQ ID NO: 7, and the second agent is Compound 4, or a pharmaceutically acceptable salt thereof.

43. The method of any one of claims 35-42, further comprising administering an additional agent selected from the group consisting of an interferon, a nucleoside analog, a nucleotide analog, a sequence specific oligonucleotide, a nucleic acid polymer, an entry inhibitor and a small molecule immunomodulator, or a pharmaceutically acceptable salt of any of the foregoing.

44. The method of claim 43, wherein the additional agent is selected from the group consisting of recombinant interferon alpha 2b, IFN-α, PEG-IFN-α-2a, lamivudine, telbivudine, adefovir dipivoxil, clevudine, entecavir, tenofovir alafenamide, and tenofovir disoproxil and an additional siRNA, or a pharmaceutically acceptable salt of any of the foregoing.

45. An improved method of treating a hepatitis B viral and/or a hepatitis D viral infection in a subject for whom therapy with a first agent selected from the group consisting of (i) an HBV surface antigen (HBsAg) reducing agent, or a pharmaceutically acceptable salt thereof, and (ii) a capsid assembly modulator (CAM), or a pharmaceutically acceptable salt thereof, has been initiated, the improved method comprising administering an effective amount of a second agent selected from the group consisting of (i) a CAM, or a pharmaceutically acceptable salt thereof, and (ii) an HBsAg reducing agent, or a pharmaceutically acceptable salt thereof, to the subject,
wherein when the first agent is an HBsAg reducing agent, or a pharmaceutically acceptable salt thereof, the second agent is a CAM, or a pharmaceutically acceptable salt thereof; and wherein when the first agent is a CAM, or a pharmaceutically acceptable salt thereof, the second agent is an HBsAg reducing agent, or a pharmaceutically acceptable salt thereof;
wherein initial administration of the second agent is after a delay period following initial administration of the first agent.

46. The improved method of claim 45, wherein the improved method is an improved method of treating a hepatitis B viral infection.

47. The improved method of claim 45, wherein the improved method is an improved method of treating a hepatitis D viral infection.

48. The improved method of any one of claims 45-47, wherein the first agent is an HBsAg reducing agent, or a pharmaceutically acceptable salt thereof; and wherein the second agent is a CAM, or a pharmaceutically acceptable salt thereof.

49. The improved method of any one of claims 45-47, wherein the first agent is a CAM, or a pharmaceutically acceptable salt thereof; and wherein the second agent is an HBsAg reducing agent, or a pharmaceutically acceptable salt thereof.

50. The improved method of any one of claims 45-49, wherein the CAM is a Class A CAM (CAM-A), or a pharmaceutically acceptable salt thereof.

51. The improved method of any one of claims 45-49, wherein the CAM is a Class E CAM (CAM-E), or a pharmaceutically acceptable salt thereof.

52. The improved method of any one of claims 45-51, wherein the HBsAg reducing agent is a small interfering RNA (siRNA) or an antisense oligonucleotide (ASO).

53. The improved method of any one of claims 45-52, wherein the CAM is a fused pyrazole compound or a fused pyrimidone compound, or a pharmaceutically acceptable salt of any of the foregoing.

54. The improved method of any one of claims 45-53, wherein the CAM is selected from the group consisting of:
N-methyl-4-[(11R)-12-[4-bromo-3-(trifluoromethyl)benzoyl]-5-(cyclopropylmethyl)-11-methyl-8-oxo-2,3,7,12-tetrazatricyclo[7.4.0.0\hat{ }2,6]trideca-1(9),3,5-trien-7-yl]benzamide (Compound 1);
4-((R)-7-(4-bromo-3-(trifluoromethyl)benzoyl)-2-(((S)-but-3-en-2-yl)amino)-6-methyl-4-oxo-5,6,7,8-tetrahydropyrido[3,4-d]pyrimidin-3(4H)-yl)-N-methylbenzamide (Compound 2);
N-[4-fluoro-3-(trifluoromethyl)phenyl]-1,3,5-trimethyl-4-[2-oxo-2-[[(1S)-1-(hydroxymethyl)-1-methyl-prop-2-ynyl]amino]acetyl]pyrrole-2-carboxamide (Compound 3);
[[2-[5-[(3-cyano-4-fluoro-phenyl)carbamoyl]-1,2,4-trimethyl-pyrrol-3-yl]-2-oxo-acetyl]-(3-ethynyloxetan-3-yl)amino]methyl dihydrogen phosphate (Compound 4);
N-(3-cyano-4-fluoro-phenyl)-4-[2-[(3-ethynyloxetan-3-yl)amino]-2-oxo-acetyl]-1,3,5-trimethyl-pyrrole-2-carboxamide (Compound 5);
(S)-2-(2-(5-((4-fluoro-3-(trifluoromethyl)phenyl)carbamoyl)-1,2,4-trimethyl-1H-pyrrol-3-yl)-2-oxoacetamido)-2-methylbut-3-yn-1-yl dihydrogen phosphate (Compound 6);
(R)-N-(2-chloropyridin-4-yl)-3-fluoro-1-methyl-4-(N-(1,1,1-trifluoropropan-2-yl)sulfamoyl)-1H-pyrrole-2-carboxamide (Compound 7);
BAY 41-4109 (Compound 8);
GLS4 (Compound 9);
NVR 3-778 (Compound 10);
RG7907 (Compound 11);
ABI-H0731 (Compound 12);
ABI-3773 (Compound 13);
ABI-4334 (Compound 14);
GLP-26 (Compound 15);
KL-060332 (Compound 16);
AB-836 (Compound 17);
VNRX-9945 (Compound 18);
(R)-N-(3-cyano-4-fluorophenyl)-1-methyl-4-(N-(1,1,1-trifluoropropan-2-yl)sulfamoyl)-1H-pyrrole-2-carboxamide (Compound 19);
JNJ-64530440 (Compound 20);
EDP-514 (Compound 21);
(S)-N-(3-cyano-4-fluorophenyl)-7-methyl-3-vinyl-3,4-dihydro-2H,7H-pyrrolo[3,4-b][1,4,5]oxathiazepine-6-carboxamide 1,1-dioxide (Compound 22);
ZM-H1505R (Compound 23);
(R)-7-(4-bromo-3-chlorobenzoyl)-2-(4-cyclopropoxyphenyl)-6-methyl-3-oxo-N-(2-(pyrimidin-4-yl)benzyl)-2,3,5,6,7,8-hexahydroimidazo[1,5-a]pyrazine-1-carboxamide (Compound 24);
(6R)-7-[4-bromo-3-(trifluoromethyl)benzoyl]-2-(3,5-dimethylpyrazol-1-yl)-6-methyl-3-(3-methylimidazo[4,5-b]pyridin-6-yl)-6,8-dihydro-5H-pyrido[3,4-d]pyrimidin-4-one (Compound 25);
4-[5-benzyl-12-(4-bromo-3-chloro-benzoyl)-8-oxo-2,3,7,12-tetrazatricyclo[7.4.0.0\hat{ }2,6]trideca-1(9),3,5-trien-7-yl]-N-methyl-benzamide (Compound 26); and
(6R)-7-[4-bromo-3-(trifluoromethyl)benzoyl]-2-(3,5-dimethylpyrazol-1-yl)-3-[4-[(2S)-2-hydroxypropoxy]phenyl]-6-methyl-6,8-dihydro-5H-pyrido[3,4-d]pyrimidin-4-one (Compound 27), or a pharmaceutically acceptable salt of any of the foregoing.

55. The improved method of any one of claims 45-54, wherein the HBsAg reducing agent is an siRNA.

56. The improved method of claim 55, wherein the siRNA is a compound selected from the group consisting of RG6346 (Roche/Dicerna), ARC-520 (Arrowhead), ARC-521 (Arrowhead), ALN-HBV (Alnylam/VIR), VIR-2218 (Alnylam/VIR), AB-729 (Arbutus), and JNJ3989 (Arrowhead/JNJ).

57. The improved method of claim 55, wherein the siRNA has a nucleic acid sequence as set forth in SEQ ID NO: 2, SEQ ID NO: 3, SEQ ID NO: 4, SEQ ID NO: 5, SEQ ID NO: 6, or SEQ ID NO: 7.

58. The improved method of any one of claims 45-54, wherein the HBsAg reducing agent is an ASO.

59. The improved method of claim 58, wherein the ASO is a compound selected from the group consisting of GSK-404 (Isis/GlaxoSmithKline), GSK-836 (Isis/GlaxoSmithKline), and RG6004 (Roche).

60. The improved method of claim 58, wherein the ASO has a nucleic acid sequence as set forth in SEQ ID NO: 1.

61. The improved method of any one of claims 45-60, wherein initial administration of the second agent occurs after the first agent has been continuously administered for at least one month.

62. The improved method of any one of claims 45-61, wherein the delay period is greater than approximately 50 days.

63. The improved method of any one of claims 45-62, wherein the delay period is greater than approximately 2 months.

64. The improved method of any one of claims 45-63, wherein the delay period is between approximately 21 days and approximately 168 days.

65. The improved method of any one of claims 45-64, wherein the delay period is between approximately 28 days and approximately 91 days.

66. The improved method of any one of claims 45-65, wherein the delay period is between approximately 8 weeks and approximately 18 weeks.

67. The improved method of any one of claims 45-66, wherein the first agent is administered at least three times at regular intervals before administration of the second agent.

68. The improved method of any one of claims 45-67, wherein the CAM is Compound 1, or a pharmaceutically acceptable salt thereof, the HBsAg reducing agent is an siRNA having the nucleic acid sequences set forth in SEQ ID NO: 2 and SEQ ID NO: 3, and the delay period comprises 50 days.

69. The improved method of any one of claims 45-68, wherein the hepatitis B viral infection is a chronic hepatitis B viral infection.

70. The improved method of any one of claims 45-69, wherein (a) the first agent is Compound 4, or pharmaceutically acceptable salt thereof, and the second agent is an siRNA having the nucleic acid sequences set forth in SEQ ID NO: 6 and SEQ ID NO: 7; or wherein (b) the first agent is an siRNA having the nucleic acid sequences set forth in SEQ ID NO: 6 and SEQ ID NO: 7, and the second agent is Compound 4, or a pharmaceutically acceptable salt thereof.

71. The improved method of any one of claims 45-70, further comprising administering an additional agent selected from the group consisting of an interferon, a nucleoside analog, a nucleotide analog, a sequence specific oligonucleotide, a nucleic acid polymer, an entry inhibitor and a small molecule immunomodulator, or a pharmaceutically acceptable salt of any of the foregoing.

72. The improved method of claim 72, wherein the additional agent is selected from the group consisting of recombinant interferon alpha 2b, IFN-α, PEG-IFN-α-2a, lamivudine, telbivudine, adefovir dipivoxil, clevudine, entecavir, tenofovir alafenamide, and tenofovir disoproxil and an additional siRNA, or a pharmaceutically acceptable salt of any of the foregoing.
